(19) United States
(12) Patent Application Publication
(10) Pub. No.: US 2024/0287122 A1
(43) Pub. Date: Aug. 29, 2024

(54) NOVEL GALACTOSIDE INHIBITOR OF GALECTINS

(71) Applicant: GALECTO BIOTECH AB, Copenhagen N (DK)

(72) Inventors: Fredrik ZETTERBERG, Copenhagen N (DK); Kristoffer PETERSON, Copenhagen N (DK)

(73) Assignee: GALECTO BIOTECH AB, Copenhagen N (DK)

(21) Appl. No.: 18/258,656

(22) PCT Filed: Dec. 20, 2021
(86) PCT No.: PCT/EP2021/086867

§ 371 Date: Jun. 21, 2023

(30) Foreign Application Priority Data
Dec. 22, 2020 (EP) 20216474.5

(51) Int. Cl. C07H 19/056; A61K 31/7056; A61K 31/706

(52) U.S. Cl. CPC C07H 19/056; A61K 31/7056; A61K 31/706

(57) ABSTRACT

D-galactopyranose compound of formula (1)

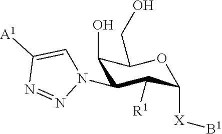

wherein
the pyranose ring is α-D-galactopyranose, and these compounds are high affinity galectin-1 and/or galectin 3 inhibitors for use in treatment of inflammation; fibrosis; scarring; keloid formation; aberrant scar formation; surgical adhesions; septic shock; cancer; metastasising cancers; autoimmune diseases, metabolic disorders; heart disease; heart failure; pathological angiogenesis; eye diseases; atherosclerosis; metabolic diseases; diabetes type I; diabetes type II; insulin resistance; Diastolic heart failure; asthma; and liver disorders.

### TECHNICAL FIELD

The present invention relates to novel compounds, the use of said compounds as medicament and for the manufacture of a medicament for the treatment of diseases or disorders such as but not limited to cancers; fibrosis; scarring; keloid formation; aberrant scar formation; surgical adhesions; pathological angiogenesis; eye diseases; HIV-1 diseases; inflammation or transplant rejection in mammals. The invention also relates to pharmaceutical compositions comprising said novel compounds.

### BACKGROUND ART

Galectins are proteins with a characteristic carbohydrate recognition domain (CRD). This is a tightly folded β-sandwich of about 130 amino acids (about 15 kDa) with the two defining features 1) a β-galactose binding site and 2) sufficient similarity in a sequence motif of about seven amino acids, most of which (about six residues) make up the β-galactose binding site. Galectins are synthesized as cytosolic proteins from where they can be targeted to the nucleus, specific cytososlic sites, or secreted to engage in mechanisms effecting physiological functions such as inflammation, immune responses, cell-migration and autophagy. (Johannes et. al 2018) There are now over 9319 publications on galectins in PubMed, with most, as mentioned above, about galectins-1 (>1989) and -3 (>4791). Evidence from literature suggests roles for galectins in e.g. fibrosis, inflammation and cancer (Dings et. al., Dubé-Delarosbil et. al 2017)

Galectin-1 is widely expressed in many cell types and tissues (www.proteinatlas.org) being involved in mechanisms such as apoptosis, adhesion and migration, cell transformation, invasion and metastasis immune escape and angiogenesis. Upregulation of galectin 1 has also been associated with cancer (Dings et. al. 2018), inflammation (Sundblad et. al., 2017) fibrotic disease (Kathiriya et. al 2017, Wu et. al. 2019 and Bennet et. al 2019) and diabetes (Drake et. al. 2022). Example of small molecule ligands including β-D-galactopyranoside were recently reviewed and exemplified in Blanchard et. al 2016 and Sethi et. al 2021).

Galectin-3 is widely expressed in many cell types and tissues (www.proteinatlas.org) being involved in mechanisms such as apoptosis, adhesion and migration, cell transformation, invasion and metastasis immune escape and angiogenesis. Upregulation of galectin 3 has also been associated with cancer, inflammation, neurodegenerative disease, fibrotic disease and diabetes (Dings et. al. 2018, Slack et. al. 2020, Li et. al. 2016) Example of small molecule ligands including β-D-galactopyranoside were recently reviewed and exemplified in Blanchard et. al 2014 and Sethi et. al 2021.

### SUMMARY OF THE INVENTION

The compounds of the present invention are novel α-D-galactopyranose compounds that unexpectedly have shown high affinity for galectin-1 and/or -3 and are considered novel potent drug candidates. It is important to emphasize that alpha and beta anomers are very different isomers and it is by no means considered to be obvious to the skilled person to expect same or similar activity of both anomers. Consequently, alpha and beta anomers do not in general posses the same activity, and this is common knowledge to the skilled person

In broad aspect the present invention concerns a D-galactopyranose compound of formula (1)

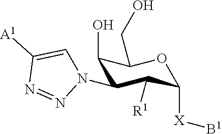

wherein
the pyranose ring is α-D-galactopyranose,
A¹ is

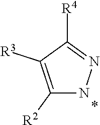

wherein the asterix * indicates the nitrogen atom of the heteroaromatic ring A¹ that is covalently attached to the triazole group of formula (1);
R² is selected from the group consisting of H; halogen; OH; CN; SH; S—C₁₋₆ alkyl; C₁₋₆ alkyl, optionally substituted with a F; cyclopropyl, optionally substituted with a F; O-cyclopropyl optionally substituted with a F; OC₁₋₆ alkyl optionally substituted with a F; NR²⁴R²⁵, wherein R²⁴ is selected from H and C₁₋₆ alkyl, and R²⁵ is selected from H, C₁₋₃ alkyl, and C(═O)R²⁶, wherein R²⁶ is selected from H, and C₁₋₆ alkyl; C(═O)NR^{24a}R^{25a}, wherein R^{24a} is selected from H and C₁₋₆ alkyl, and R^{25a} is selected from H, C₁₋₃ alkyl, and C(═O)R^{26a}, wherein R^{26a} is selected from H, and C₁₋₆ alkyl; C(═O)OR^{24b}R^{25b}, wherein R^{24b} is selected from H and C₁₋₆ alkyl, and R^{25b} is selected from H, C₁₋₃ alkyl, and C(═O)R^{26b}, wherein R^{26b} is selected from H, and C₁₋₆ alkyl;
R³ is selected from the group consisting of H; halogen; OH; CN; SH; S—C₁₋₆ alkyl; C₁₋₆ alkyl, optionally substituted with a F; cyclopropyl, optionally substituted with a F; O-cyclopropyl optionally substituted with a F; OC₁₋₆ alkyl optionally substituted with a F; NR²⁴R²⁵, wherein R²⁴ is selected from H and C₁₋₆ alkyl, and R²⁵ is selected from H, C₁₋₃ alkyl, and C(═O)R²⁶, wherein R²⁶ is selected from H, and C₁₋₆ alkyl; C(═O)NR^{24a}R^{25a}, wherein R^{24a} is selected from H and C₁₋₆ alkyl, and R^{25a} is selected from H, C₁₋₃ alkyl, and C(═O)R^{26a}, wherein R^{26a} is selected from H, and C₁₋₆ alkyl; C(═O)OR^{24b}R^{25b}, wherein R^{24b} is selected from H and C₁₋₆ alkyl, and R^{25b} is selected from H, C₁₋₃ alkyl, and C(═O)R^{26b}, wherein R^{26b} is selected from H, and C₁₋₆ alkyl;
R⁴ is selected from the group consisting of H; halogen; OH; CN; SH; S—C₁₋₆ alkyl; C₁₋₆ alkyl, optionally substituted with a F; cyclopropyl, optionally substituted with a F; O-cyclopropyl optionally substituted with a F; OC₁₋₆ alkyl optionally substituted with a F; NR²⁴R²⁵, wherein R²⁴ is selected from H and C₁₋₆ alkyl, and R²⁵ is selected from H, C₁₋₃ alkyl, and C(═O)R²⁶, wherein R²⁶ is selected from H, and C₁₋₆ alkyl; C(═O)NR^{24a}R^{25a}, wherein R^{24a} is selected from H and C₁₋₆ alkyl, and R^{25a} is selected from H, C₁₋₃ alkyl, and C(═O)R^{26a}, wherein R^{26a} is selected from H, and C₁₋₆ alkyl; C(═O)OR^{24b}R^{25b}, wherein R^{24b} is selected from H and C₁₋₆ alkyl, and R^{25b} is selected from H, C₁₋₃ alkyl, and C(═O)R^{26b}, wherein R^{26b} is selected from H, and C₁₋₆ alkyl;
X is selected from S, Se, SO, SO₂, O, C═O, and CR^{2a}R^{3a} wherein R^{2a} and R^{3a} are independently selected from hydrogen, OH, or halogen;
B¹ is selected from a) a C₁₋₆ alkyl or branched C₃₋₆ alkyl substituted with a five or six membered heteroaromatic ring, optionally substituted with a substituent selected from CN, a halogen, methyl optionally substituted with a F, OCH₃ optionally substituted with a F, OCH₂CH₃ optionally substituted with a F, OH, and R^{4a}—CONH— wherein R^{4a} is selected from C₁₋₃ alkyl and cyclopropyl; or a C₁₋₆ alkyl substituted with a phenyl, optionally substituted with a substituent selected from CN, a halogen, methyl optionally substituted with a F, OCH₃ optionally substituted with a F, OCH₂CH₃ optionally substituted with a F, OH, and R^{5a}—CONH— wherein R^{5a} is selected from C₁₋₃ alkyl and cyclopropyl; b) an aryl, such as phenyl or naphthyl, optionally substituted with a group selected from a halogen; a spiro heterocycle, such as N-(2-oxa)-6-azaspiro[3.3]heptanyl; C₂-alkynyl; CN; —COOH; COOC₁₋₄ alkyl; —CONR⁶R⁷, wherein R⁶ and R⁷ are independently selected from H, C₁₋₃ alkyl, cyclopropyl, and iso-propyl, or R⁶ and R⁷ together with the nitrogen form a heterocycloalkyl; C₁₋₃ alkyl, optionally substituted with a F; cyclopropyl, optionally substituted with a F; isopropyl, optionally substituted with a F; SC₁₋₃ alkyl, optionally substituted with a F; OC₁₋₃ alkyl, optionally substituted with a F; O-cyclopropyl, optionally substituted with a F; O-isopropyl, optionally substituted with a F; NR⁸R⁹, wherein R⁸ and R⁹ are independently selected from H, C₁₋₃ alkyl and isopropyl; OH; and R¹⁰—CONH— wherein R¹⁰ is selected from C₁₋₃ alkyl and cyclopropyl; an aryl; and a heterocycle, c) a C₅₋₇ cycloalkyl, optionally substituted with a substituent selected from a halogen, C₂-alkynyl, CN, methyl optionally substituted with a F, OCH₃ optionally substituted with a F, OCH₂CH₃ optionally substituted with a F, OH, and R¹¹—CONH— wherein R¹¹ is selected from C₁₋₃ alkyl and cyclopropyl; d) a heterocycle, such as heteroaryl or heterocycloalkyl, optionally substituted with a group selected from a halogen; a spiro heterocycle, such as N-(2-oxa)-6-azaspiro[3.3]heptanyl; C₂-alkynyl; CN; —COOH; COOC₁₋₄ alkyl; —CONR¹²R¹³, wherein R¹² and R¹³ are independently selected from H, C₁₋₃ alkoxy, branched C₃_6 alkyl, C₁₋₆ alkyl optionally substituted with a F, bicyclopentanyl, CH₂-cyclopropyl, and CH₂-cyclobutyl, or R¹² and R¹³ together with the nitrogen form a heterocycloalkyl; C₁₋₃ alkyl, optionally substituted with a F; cyclopropyl, optionally substituted with a F; isopropyl, optionally substituted with a F; SC₁₋₃ alkyl, optionally substituted with a F; OC₁₋₃ alkyl, optionally substituted with a F; O-cyclopropyl, optionally substituted with a F; O-isopropyl, optionally substituted with a F; SC₁₋₃ alkyl, optionally substituted with a F; NR¹⁴R¹⁵, wherein R¹⁴ and R¹⁵ are independently selected from H, C₁₋₃ alkyl and isopropyl; OH; an aryl; a heterocycle; and R¹⁶—CONH— wherein R¹⁶ is selected from C₁₋₃ alkyl and cyclopropyl; e) a C₁₋₆ alkyl or branched C₃₋₆ alkyl; and f) C₂_₆ alkynyl
R¹ is selected from the group consisting of a) H, b) OH, c) OC₁₋₆ alkyl optionally substituted with one or more halogen, phenyl, phenyl substituted with one or more groups selected form OH and halogen, CN, OR¹⁷, NR¹⁸R¹⁹, and CONH₂, wherein R¹⁷ is selected from the group consisting of H, CN, a halogen, methyl optionally substituted with a F, OCH₃ optionally substituted with a F, OCH₂CH₃ optionally substituted with a F, OH, and R²⁰—CONH— wherein R²⁰ is selected from C₁-3 alkyl and cyclopropyl, R¹⁸ is selected from the group consisting of H, CN, a halogen, methyl optionally substituted with a F, OCH₃ optionally substituted with a F, OCH₂CH₃ optionally substituted with a F, OH, and R²¹—CONH— wherein R²¹ is selected from C₁₋₃ alkyl and cyclopropyl, and R¹⁹ is selected from the group consisting of H, CN, a halogen, methyl optionally substituted with a F, OCH₃ optionally substituted with a F, OCH₂CH₃ optionally substituted with a F, OH, and R²²—CONH— wherein R²² is selected from C₁₋₃ alkyl and cyclopropyl, d) branched OC₃₋₆ alkyl optionally substituted with one or more halogen, CN, OR²³, NR²⁴R²⁵, and CONH₂, wherein R²³ is selected from the group consisting of H, CN, a halogen, methyl optionally substituted with a F, OCH₃ optionally substituted with a F, OCH₂CH₃ optionally substituted with a F, OH, and R²⁶—CONH— wherein R²⁶ is selected from C₁₋₃ alkyl and cyclopropyl, R²⁴ is selected from the group consisting of H, CN, a halogen, methyl optionally substituted with a F, OCH₃ optionally substituted with a F, OCH₂CH₃ optionally substituted with a F, OH, and R²⁷—CONH— wherein R²⁷ is selected from C₁₋₃ alkyl and cyclopropyl, and R²⁵ is selected from the group consisting of H, CN, a halogen, methyl optionally substituted with a F, OCH₃ optionally substituted with a F, OCH₂CH₃ optionally substituted with a F, OH, and R²⁸—CONH— wherein R²⁸ is selected from C₁₋₃ alkyl and cyclopropyl, and e) cyclic OC₃₋₆ alkyl optionally substituted with one or more halogen, CN, OR²⁹, NR³⁰R³¹, and CONH₂, wherein R²⁹ is selected from the group consisting of H, CN, a halogen, methyl optionally substituted with a F, OCH₃ optionally substituted with a F, OCH₂CH₃ optionally substituted with a F, OH, and R³²—CONH— wherein R³² is selected from C₁₋₃ alkyl and cyclopropyl, R³⁰ is selected from the group consisting of H, CN, a halogen, methyl optionally substituted with a F, OCH₃ optionally substituted with a F, OCH₂CH₃ optionally substituted with a F, OH, and R³³—CONH— wherein R³³ is selected from C₁-3 alkyl and cyclopropyl, and R³¹ is selected from the group consisting of H, CN, a halogen, methyl optionally substituted with a F, OCH₃ optionally substituted with a F, OCH₂CH₃ optionally substituted with a F, OH, and R³⁴—CONH— wherein R³⁴ is selected from C₁₋₃ alkyl and cyclopropyl; or a pharmaceutically acceptable salt or solvate thereof.

In a further aspect the present invention concerns a D-galactopyranose compound of formula (1)

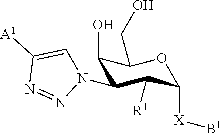

wherein
the pyranose ring is α-D-galactopyranose,
A¹ is

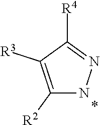

wherein the asterix * indicates the nitrogen atom of the heteroaromatic ring A¹ that is covalently attached to the triazole group of formula (1);
R² is selected from the group consisting of H; halogen; OH; CN; SH; S—C₁₋₆ alkyl; C₁₋₆ alkyl, optionally substituted with a F; cyclopropyl, optionally substituted with a F; O-cyclopropyl optionally substituted with a F; OC₁₋₆ alkyl optionally substituted with a F; NR²⁴R²⁵, wherein R²⁴ is selected from H and C₁₋₆ alkyl, and R²⁵ is selected from H, C₁₋₃ alkyl, and C(═O)R²⁶, wherein R²⁶ is selected from H, and C₁₋₆ alkyl; C(═O)NR^{24a}R^{25a}, wherein R^{24a} is selected from H and C₁₋₆ alkyl, and R^{25a} is selected from H, C₁₋₃ alkyl, and C(═O)R^{26a}, wherein R^{26a} is selected from H, and C₁₋₆ alkyl; C(═O)OR^{24b}R^{25b}, wherein R^{24b} is selected from H and C₁₋₆ alkyl, and R^{25b} is selected from H, C₁₋₃ alkyl, and C(═O)R^{26b}, wherein R^{26b} is selected from H, and C₁₋₆ alkyl;
R³ is selected from the group consisting of H; halogen; OH; CN; SH; S—C₁₋₆ alkyl; C₁₋₆ alkyl, optionally substituted with a F; cyclopropyl, optionally substituted with a F; O-cyclopropyl optionally substituted with a F; OC₁₋₆ alkyl optionally substituted with a F; NR²⁴R²⁵, wherein R²⁴ is selected from H and C₁₋₆ alkyl, and R²⁵ is selected from H, C₁₋₃ alkyl, and C(═O)R²⁶, wherein R²⁶ is selected from H, and C₁₋₆ alkyl; C(═O)NR^{24a}R^{25a}, wherein R^{24a} is selected from H and C₁₋₆ alkyl, and R^{25a} is selected from H, C₁₋₃ alkyl, and C(═O)R^{26a}, wherein R^{26a} is selected from H, and C₁₋₆ alkyl; C(═O)OR^{24b}R^{25b}, wherein R^{24b} is selected from H and C₁₋₆ alkyl, and R^{25b} is selected from H, C₁₋₃ alkyl, and C(═O)R^{26b}, wherein R^{26b} is selected from H, and C₁₋₆ alkyl;
R⁴ is selected from the group consisting of H; halogen; OH; CN; SH; S—C₁₋₆ alkyl; C₁₋₆ alkyl, optionally substituted with a F; cyclopropyl, optionally substituted with a F; O-cyclopropyl optionally substituted with a F; OC₁₋₆ alkyl optionally substituted with a F; NR²⁴R²⁵, wherein R²⁴ is selected from H and C₁₋₆ alkyl, and R²⁵ is selected from H, C₁₋₃ alkyl, and C(═O)R²⁶, wherein R²⁶ is selected from H, and C₁₋₆ alkyl; C(═O)NR^{24a}R^{25a}, wherein R^{24a} is selected from H and C₁₋₆ alkyl, and R^{25a} is selected from H, C₁₋₃ alkyl, and C(═O)R^{26a}, wherein R^{26a} is selected from H, and C₁₋₆ alkyl; C(═O)OR^{24b}R^{25b}, wherein R^{24b} is selected from H and C₁₋₆ alkyl, and R^{25b} is selected from H, C₁₋₃ alkyl, and C(═O)R^{26b}, wherein R^{26b} is selected from H, and C₁₋₆ alkyl;
X is selected from S, Se, SO, SO₂, O, C═O, and CR^{2a}R^{3a} wherein R^{2a} and R^{3a} are independently selected from hydrogen, OH, or halogen;
B¹ is selected from a) a C₁₋₆ alkyl or branched C₃₋₆ alkyl substituted with a five or six membered heteroaromatic ring, optionally substituted with a substituent selected from CN, a halogen, methyl optionally substituted with a F, OCH₃ optionally substituted with a F, OCH₂CH₃ optionally substituted with a F, OH, and R^{4a}—CONH— wherein R^{4a} is selected from C₁₋₃ alkyl and cyclopropyl; or a C₁₋₆ alkyl substituted with a phenyl, optionally substituted with a substituent selected from CN, a halogen, methyl optionally substituted with a F, OCH₃ optionally substituted with a F, OCH₂CH₃ optionally substituted with a F, OH, and R^{5a}—CONH— wherein R^{5a} is selected from C₁₋₃ alkyl and cyclopropyl; b) an aryl, such as phenyl or naphthyl, optionally substituted with a group selected from a halogen; a spiro heterocycle, such as N-(2-oxa)-6-azaspiro[3.3]heptanyl; C₂-alkynyl; CN; —COOH; COOC₁₋₄ alkyl; —CONR⁶R⁷, wherein R⁶ and R⁷ are independently selected from H, C₁₋₃ alkyl, cyclopropyl, and iso-propyl, or R⁶ and R⁷ together with the nitrogen form a heterocycloalkyl; C₁₋₃ alkyl, optionally substituted with a F; cyclopropyl, optionally substituted with a F; isopropyl, optionally substituted with a F; SC₁₋₃ alkyl, optionally substituted with a F; OC₁₋₃ alkyl, optionally substituted with a F; O-cyclopropyl, optionally substituted with a F; O-isopropyl, optionally substituted with a F; NR⁸R⁹, wherein R⁸ and R⁹ are independently selected from H, C₁₋₃ alkyl and isopropyl; OH; and R¹⁰—CONH— wherein R¹⁰ is selected from C₁₋₃ alkyl and cyclopropyl; an aryl; and a heterocycle, c) a C₅-7 cycloalkyl, optionally substituted with a substituent selected from a halogen, C₂-alkynyl, CN, methyl optionally substituted with a F, OCH₃ optionally substituted with a F, OCH₂CH₃ optionally substituted with a F, OH, and R¹¹—CONH— wherein R¹¹ is selected from C₁₋₃ alkyl and cyclopropyl; d) a heterocycle, such as heteroaryl or heterocycloalkyl, optionally substituted with a group selected from a halogen; a spiro heterocycle, such as N-(2-oxa)-6-azaspiro[3.3]heptanyl; C₂-alkynyl; CN; —COOH; COOC₁₋₄ alkyl; —CONR¹²R¹³, wherein R¹² and R¹³ are independently selected from H, C₁₋₃ alkyl, cyclopropyl, and iso-propyl or R¹² and R¹³ together with the nitrogen form a heterocycloalkyl; C₁_3 alkyl, optionally substituted with a F; cyclopropyl, optionally substituted with a F; isopropyl, optionally substituted with a F; SC₁₋₃ alkyl, optionally substituted with a F; OC₁₋₃ alkyl, optionally substituted with a F; O-cyclopropyl, optionally substituted with a F; O-isopropyl, optionally substituted with a F; SC₁₋₃ alkyl, optionally substituted with a F; NR¹⁴R¹⁵, wherein R¹⁴ and R¹⁵ are independently selected from H, C₁₋₃ alkyl and isopropyl; OH; an aryl; a heterocycle; and R¹⁶—CONH— wherein R¹⁶ is selected from C₁₋₃ alkyl and cyclopropyl; e) a C₁₋₆ alkyl or branched C₃₋₆ alkyl; and f) C₂-6 alkynyl
R¹ is selected from the group consisting of a) H, b) OH, c) OC₁₋₆ alkyl optionally substituted with one or more halogen, phenyl, phenyl substituted with one or more groups selected form OH and halogen, CN, OR¹⁷, NR¹⁸R¹⁹, and CONH₂, wherein R¹⁷ is selected from the group consisting of H, CN, a halogen, methyl optionally substituted with a F, OCH₃ optionally substituted with a F, OCH₂CH₃ optionally substituted with a F, OH, and R²⁰—CONH— wherein R²⁰ is selected from C₁₋₃ alkyl and cyclopropyl, R¹⁸ is selected from the group consisting of H, CN, a halogen, methyl optionally substituted with a F, OCH₃ optionally substituted with a F, OCH₂CH₃ optionally substituted with a F, OH, and R²¹—CONH— wherein R²¹ is selected from C₁₋₃ alkyl and cyclopropyl, and R¹⁹ is selected from the group consisting of H, CN, a halogen, methyl optionally substituted with a F, OCH₃ optionally substituted with a F, OCH₂CH₃ optionally substituted with a F, OH, and R²²—CONH— wherein R²² is selected from C₁₋₃ alkyl and cyclopropyl, d) branched OC₃₋₆ alkyl optionally substituted with one or more halogen, CN, OR²³, NR²⁴R²⁵, and CONH₂, wherein R²³ is selected from the group consisting of H, CN, a halogen, methyl optionally substituted with a F, OCH₃ optionally substituted with a F, OCH₂CH₃ optionally substituted with a F, OH, and R²⁶—CONH— wherein R²⁶ is selected from C₁₋₃ alkyl and cyclopropyl, R²⁴ is selected from the group consisting of H, CN, a halogen, methyl optionally substituted with a F, OCH₃ optionally substituted with a F, OCH₂CH₃ optionally substituted with a F, OH, and R²⁷—CONH— wherein R²⁷ is selected from C₁₋₃ alkyl and cyclopropyl, and R²⁵ is selected from the group consisting of H, CN, a halogen, methyl optionally substituted with a F, OCH₃ optionally substituted with a F, OCH₂CH₃ optionally substituted with a F, OH, and R²⁸—CONH— wherein R²⁸ is selected from C₁-3 alkyl and cyclopropyl, and e) cyclic OC₃₋₆ alkyl optionally substituted with one or more halogen, CN, OR²⁹, NR³⁰R³¹, and CONH₂, wherein R²⁹ is selected from the group consisting of H, CN, a halogen, methyl optionally substituted with a F, OCH₃ optionally substituted with a F, OCH₂CH₃ optionally substituted with a F, OH, and R³²—CONH— wherein R³² is selected from C₁₋₃ alkyl and cyclopropyl, R³⁰ is selected from the group consisting of H, CN, a halogen, methyl optionally substituted with a F, OCH₃ optionally substituted with a F, OCH₂CH₃ optionally substituted with a F, OH, and R³³—CONH— wherein R³³ is selected from C₁-3 alkyl and cyclopropyl, and R³¹ is selected from the group consisting of H, CN, a halogen, methyl optionally substituted with a F, OCH₃ optionally substituted with a F, OCH₂CH₃ optionally substituted with a F, OH, and R³⁴—CONH— wherein R³⁴ is selected from C₁-3 alkyl and cyclopropyl; or a pharmaceutically acceptable salt or solvate thereof.

In an embodiment R² is hydrogen. In another embodiment R² is a C₁₋₃ alkyl. In a further embodiment R² is a halogen. In an embodiment R³ is hydrogen. In another embodiment R³ is a C₁₋₃ alkyl. In a further embodiment R³ is a halogen. In a further embodiment R⁴ is a halogen, such as Cl or F. In a still further embodiment R⁴ is a C₁-3 alkyl. In a further embodiment R⁴ is a C₁-3 alkyl substituted with a F, such as CF₃.

In a still further embodiment X is selected from S.

In a further embodiment B1 is selected from a heteroaryl, optionally substituted with a group selected from a halogen; C₂-alkynyl; CN; methyl optionally substituted with a F; a spiro heterocycle; SC₁₋₃ alkyl, optionally substituted with a F; a CONR¹²R¹³, wherein R¹² and R¹³ are independently selected from H, C₁₋₃ alkyl, cyclopropyl, and iso-propyl or R¹² and R¹³ together with the nitrogen form a heterocycloalkyl; and a heterocycle, such as a tetrahydropyridin. Typically, B1 is selected from a pyridinyl, optionally substituted with a group selected from a Cl; Br; F; ethynyl; N-(2-oxa)-6-azaspiro[3.3]heptanyl; CO-azetidinyl; CONHCH₃; CONHCH₂CH₃; CON(CH₃)₂; CN; methyl; SCH₃; SCF₃; CF₃; imidazole; pyridin; pyrimidin; oxazol; and thiazol; such as pyridinyl substituted with one or two selected from Cl, Br, CN, and CONHCH₃. In a further embodiment B1 is selected from a pyridinyl, optionally substituted with a group selected from a Cl; Br; F; ethynyl; N-(2-oxa)-6-azaspiro[3.3]heptanyl; CO-azetidinyl; CONHCH₃; CONHCH₂CH₃; CON(CH(CH₃)₂)(CH₂CH₃); CON(isobutyl)₂; CON(CH₃)(CH₂C(CH₃)₂F); CON(CH₂CH₃)(CH₂C(CH₃)₂F); CON(CH₂CH₃)(CH₂-cyclopropyl); CON(CH₂CH₃)(tert-butyl); CON(CH₂-cyclopropyl)₂; CON(CH₂CH₃)(CH₂-cyclobutyl); CON(CH(CH₃)₂)(CH₂-cyclobutyl); CON(CH₂-cyclobutyl)₂; CON(CH₂CH₃)(CH₂CF₃); CON(CH(CH₃)₂)(CH₂-cyclopropyl); CON(CH(CH₃)₂)(isobutyl); CON(CH₃)₂; CO-pyrrolidinyl; CON(OCH₃)(CH₂-cyclopropyl); CONHCH₂CH₂CH₂CH₃; CONH(isobutyl); CONH(CH₂CH₂F); CONH(bicyclopentanyl); CONH(cyclopropyl); CONH(cyclobutyl); CN; methyl; SCH₃; SCF₃; CF₃; imidazole; pyridin; pyrimidin; oxazol; and thiazol.

In a still further embodiment B1 is selected from a phenyl, optionally substituted with a group selected from a halogen; CN; —CONR⁶R⁷, wherein R⁶ and R⁷ are independently selected from H, C₁₋₃ alkyl, cyclopropyl, and iso-propyl; and C₁₋₃ alkyl, optionally substituted with a F. Typically, B1 is selected from a phenyl, optionally substituted with a group selected from a Cl; F; Br; CN; CONHCH₃; and C₁₋₃ alkyl, optionally substituted with a F; such as phenyl substituted with one or two selected from Cl, Br, CN, and CONHCH₃.

In a further embodiment R¹ is selected from H, OH, OC₁₋₄ alkyl, such as O-methyl, O-ethyl, or O-isopropyl, OC₁₋₄ alkyl substituted with at least one from the group consisting of phenyl and phenyl substituted with one or more groups selected form OH and halogen. Typically, R¹ is selected from H, OH, OCH₃, and OC₁₋₆ alkyl optionally
substituted with one or more halogen; such as OH and OCH₃.
Preferably the D-galactopyranose compound of formula (1) is selected form any one of the group consisting of:
5-Bromo-2-(N-methyl-carbonyl)phenyl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-cyanophenyl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-cyanopyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Chloropyridin-3-yl 3-deoxy-3-[4-(3-fluoro-1H-1,2-pyrazol-1-yl)-1H-1,2,3-triazol-1-yl]-1-thio-α-D-galactopyranoside, and
5-Chloropyridin-3-yl 3-[4-(3-chloro-1H-1,2-pyrazol-1-yl)-1H-1,2,3-triazol-1-yl]-3-deoxy-1-thio-α-D-galactopyranoside, or
a pharmaceutically acceptable salt or solvate thereof.

In a further embodiment the D-galactopyranose compound of formula (1) is selected form any one of the group consisting of:
5-Chloro-2-(trifluoromethyl)pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-(trifluoromethyl)pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
3-Chloro-2-(trifluoromethyl)pyridin-5-yl 3-[4-(3-chloro-1H-1,2-pyrazol-1-yl)-1H-1,2,3-triazol-1-yl]-3-deoxy-2-O-methyl-1-thio-α-D-galactopyranoside,
3-Bromo-2-(trifluoromethyl)pyridin-5-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-(N,N-dimethylcarbamoyl)pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-cyanopyridin-3-yl 3-[4-(4-chloro-1H-1,2-pyrazol-1-yl)-1H-1,2,3-triazol-1-yl]-3-deoxy-2-O-methyl-1-thio-α-D-galactopyranoside,
5-Bromo-2-cyanopyridin-3-yl 3-deoxy-3-[4-(3-fluoro-1H-1,2-pyrazol-1-yl)-1H-1,2,3-triazol-1-yl]-2-O-methyl-1-thio-α-D-galactopyranoside,
5-Bromo-2-cyanopyridin-3-yl 3-deoxy-3-[4-(4-fluoro-1H-1,2-pyrazol-1-yl)-1H-1,2,3-triazol-1-yl]-2-O-methyl-1-thio-α-D-galactopyranoside,
5-Bromo-2-cyanopyridin-3-yl 3-deoxy-3-[4-(3-methyl-1H-1,2-pyrazol-1-yl)-1H-1,2,3-triazol-1-yl]-2-O-methyl-1-thio-α-D-galactopyranoside,
5-Bromo-2-cyanopyridin-3-yl 3-deoxy-3-[4-(5-methyl-1H-1,2-pyrazol-1-yl)-1H-1,2,3-triazol-1-yl]-2-O-methyl-1-thio-α-D-galactopyranoside,
5-Bromo-2-cyanopyridin-3-yl 3-[4-(3-chloro-5-methyl-1H-1,2-pyrazol-1-yl)-1H-1,2,3-triazol-1-yl]-3-deoxy-2-O-methyl-1-thio-α-D-galactopyranoside,
5-Bromo-2-cyanopyridin-3-yl 3-{4-[5-chloro-3-(trifluoromethyl)-1H-1,2-pyrazol-1-yl]-1H-1,2,3-triazol-1-yl}-3-deoxy-2-O-methyl-1-thio-α-D-galactopyranoside,
5-Bromo-2-cyanopyridin-3-yl 3-[4-(3-chloro-4-methyl-1H-1,2-pyrazol-1-yl)-1H-1,2,3-triazol-1-yl]-3-deoxy-2-O-methyl-1-thio-α-D-galactopyranoside,
5-Bromo-2-(N,N-ethylisopropylcarbamoyl)pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-(N,N-diisobutylcarbamoyl)pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-[N,N-(cyclopropylmethyl)ethylcarbamoyl]pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-[N,N-(2-fluoro-2-methylpropyl)methylcarbamoyl]pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-[N,N-(tert-butyl)ethylcarbamoyl]pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-[N,N-bis(cyclopropylmethyl)carbamoyl]pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-[N,N-(cyclobutylmethyl)ethylcarbamoyl}pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-[N,N-(cyclobutylmethyl)isopropylcarbamoyl]pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-[N,N-bis(cyclobutylmethyl)carbamoyl]pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-(pyrrolidine-1-carbonyl)pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-[N,N-ethyl(2,2,2-trifluoroethyl)carbamoyl]pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-[N,N-ethyl(2-fluoro-2-methylpropyl)carbamoyl]pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-[N,N-(cyclopropylmethyl)isopropylcarbamoyl]pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-(N,N-isobutylisopropylcarbamoyl)pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-[N,N-(cyclopropylmethyl)methoxycarbamoyl]pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-(N-methylcarbamoyl)pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-(N-ethylcarbamoyl)pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-(N-butylcarbamoyl)pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-(N-isobutylcarbamoyl)pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-[N-(2-fluoroethyl)carbamoyl]pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-(N-bicyclo[1.1.1]pentan-1-ylcarbamoyl)pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-(N-cyclobutylcarbamoyl)pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-(N-cyclopropylcarbamoyl)pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-cyanopyridin-3-yl 3-[4-(3,4-dichloro-1H-1,2-pyrazol-1-yl)-1H-1,2,3-triazol-1-yl]-3-deoxy-2-O-methyl-1-thio-α-D-galactopyranoside,
5-Bromo-2-cyanopyridin-3-yl 3-[4-(3-chloro-4-fluoro-1H-1,2-pyrazol-1-yl)-1H-1,2,3-triazol-1-yl]-3-deoxy-2-O-methyl-1-thio-α-D-galactopyranoside; or
a pharmaceutically acceptable salt or solvate thereof.

In a further aspect the present invention relates to a compound of formula (1) for use as a medicine.

In a still further aspect, the present invention relates to a pharmaceutical composition comprising the compound of any one of the previous claims and optionally a pharmaceutically acceptable additive, such as a carrier and/or excipient.

In a further aspect the present invention relates to a compound of formula (1) of the present invention for use in a method for treating a disease or disorder relating to the binding of a galectin-1 and/or a galectin 3 to a ligand in a mammal, such as a human.

In a further embodiment the disease or disorder is selected from the group consisting of inflammation, such as acute post myocardial infarctions (MI), acute coronary syndrome, acute stent occlusion, acute myocardial reperfusion injury, acute pneumonitidies, acute lung injury (ALI), acute kidney injury (AKI), acute hepatitis, acute on chronic liver failure, acute alcohol hepatitis, acute pancreatitis, acute uveitis, acute pancreatitis related liponecrosis, acute retinitis, acute nephritis, acute myocarditis, chronic autoimmune diseases in all organs, (e.g. lung, liver, kidney, heart, skin, muscle, gut), chronic bacterial infections, chronic viral related inflammation; fibrosis, such as pulmonary fibrosis, liver fibrosis, kidney fibrosis, ophthalmological fibrosis and fibrosis of the skin and heart, acute post-surgical ocular fibrosis, acute transplantation rejection of the kidney, heart, lung, liver, and pancreas, acute post explosion/improvised explosive devices, acute post toxic dust (such as dust from terror attack known as 9/11), acute chemical exposure, chronic lung fibrosis, interstitial lung fibrosis (IPF), Interstitial Lung Disease (ILD), Childhood ILD (ChILD); chronic liver fibrosis, chronic alcohol fibrosis, chronic viral fibrosis, chronic diabetic fibrosis, diabetic nephropathy, chronic glomerulonephritis, renal artery stenosis, endometriosis; scarring; keloid formation; aberrant scar formation; surgical adhesions; scleroderma; systemic sclerosis; septic shock; cancers, such as carcinomas, sarcomas, leukemias and lymphomas, such as T-cell lymphomas; metastasising cancers; autoimmune diseases, such as psoriasis, rheumatoid arthritis, Crohn's disease, ulcerative colitis, intestinal fibrosis, ankylosing spondylitis, systemic lupus erythematosus; metabolic disorders; coagulopathies, such as thrombosis proneness idiopathic (thrombophilia), autoimmune based thrombophilia, microthrombosis at multiorgan failure, COVID-19 related coagulopathy, thrombophilia in cancer disease; cardiovascular disorders, such as cardiac fibrosis, cardiac failure, left and right atrial fibrillation, atheromatosis, arterial inflammation, arterial calcification, aortic stenosis; heart disease; heart failure; aortic stenosis, atherosclerosis, pathological angiogenesis, such as ocular angiogenesis or a disease or condition associated with ocular angiogenesis, e.g. neovascularization related to cancer; and eye diseases, such as age-related macular degeneration and corneal neovascularization; atherosclerosis; endocrine disorders, such as Addison, autoimmune hypophysitis; metabolic diseases such as diabetes; type 2 diabetes; insulin resistance; obesity; Diastolic HF; atrophic diseases in the brain, such as Alzheimer's and Parkinson's, atrophic diseases in the cerebellum, such as cerebellar atrophy, atrophic spinal diseases such as ALS; disorders related to transplantation in organs, such as anti-rejection prophylaxis, anti-acute rejection, anti-chronic rejection; acute burn; acute inflammatory reaction; chronic acute skin graft rejection; chronic scarring; asthma and other interstitial lung diseases, including Hermansky-Pudlak syndrome, pulmonary arterial hypertension, Rheumatoid disease associated interstitial lung disease RA-ILD, Systemic Sclerosis SSc-ILD, lung disease with fibrosis such as COPD (Chronic Obstructive Pulmonary Disease) and asthma; Otosclerosis, mesothelioma; post-surgery disorders, such as anti-keloid, anti-stricture, anti-adhesion, anti-thrombosis, fibrosis/scar reduction following cosmetic procedures; toxin exposure disorders, such as toxic hepatitis, cholera toxin related, mushroom toxin based acute renal failure, pertussis toxin, Aeromonas hydrophila enterotoxin, cadmium induced cardiac toxicity, helicobacter O-antigen related toxicity, LPS based toxicity, Streptozotocin toxicity, asbestos exposure, Nephrogenic Systemic Fibrosis (Post Contrast Agents); Tissue injury, such as Spinal cord injury, Peripheral nerve repair; congenital hepatic fibrosis; hereditary fibrosing poikiloderma with tendon contractures, myopathy, and pulmonary fibrosis; liver disorders, such as non-alcoholic steatohepatitis (NASH) or non-alcoholic fatty liver disease, liver cirrhosis of various origins, such as alcoholic and non-alcoholic, autoimmune cirrhosis such as primary biliary cirrhosis and sclerosing cholangitis, virally induced cirrhosis, cirrhosis induced by genetic disease; Liver cancer, cholangiocarcinoma, biliary tract cancer; neurodegenerative disorders such as Parkinsons disease, Alzheimers disease, cognitive impairment, cerebrovascular diseases such as stroke, traumatic brain injury, Huntington's disease, amyotrophic lateral sclerosis (ALS), multiple sclerosis (MS), peripheral nephropathy, in a mammal, such as a human.

In a still further aspect the present invention relates to a method for treatment of a disease or disorder relating to the binding of a galectin-1 and/or -3 to a ligand in a mammal, such as a human, wherein a therapeutically effective amount of at least one compound of formula (1) of the present invention is administered to a mammal in need of said treatment.

In a further embodiment the disease or disorder is selected from the group consisting of inflammation, such as acute post myocardial infarctions (MI), acute coronary syndrome, acute stent occlusion, acute myocardial reperfusion injury, acute pneumonitidies, acute lung injury (ALI), acute kidney injury (AKI), acute hepatitis, acute on chronic liver failure, acute alcohol hepatitis, acute pancreatitis, acute uveitis, acute pancreatitis related liponecrosis, acute retinitis, acute nephritis, acute myocarditis, chronic autoimmune diseases in all organs, (e.g. lung, liver, kidney, heart, skin, muscle, gut), chronic bacterial infections, chronic viral related inflammation; fibrosis, such as pulmonary fibrosis, liver fibrosis, kidney fibrosis, ophthalmological fibrosis and fibrosis of the skin and heart, acute post-surgical ocular fibrosis, acute transplantation rejection of the kidney, heart, lung, liver, and pancreas, acute post explosion/improvised explosive devices, acute post toxic dust (such as dust from terror attack known as 9/11), acute chemical exposure, chronic lung fibrosis, interstitial lung fibrosis (IPF), Interstitial Lung Disease (ILD), Childhood ILD (ChILD); chronic liver fibrosis, chronic alcohol fibrosis, chronic viral fibrosis, chronic diabetic fibrosis, diabetic nephropathy, chronic glomerulonephritis, renal artery stenosis, endometriosis; scarring; keloid formation; aberrant scar formation; surgical adhesions; scleroderma; systemic sclerosis; septic shock; cancers, such as carcinomas, sarcomas, leukemias and lymphomas, such as T-cell lymphomas; metastasising cancers; autoimmune diseases, such as psoriasis, rheumatoid arthritis, Crohn's disease, ulcerative colitis, intestinal fibrosis, ankylosing spondylitis, systemic lupus erythematosus; metabolic disorders; coagulopathies, such as thrombosis proneness idiopathic (thrombophilia), autoimmune based thrombophilia, microthrombosis at multiorgan failure, COVID-19 related coagulopathy, thrombophilia in cancer disease; cardiovascular disorders, such as cardiac fibrosis, cardiac failure, left and right atrial fibrillation, atheromatosis, arterial inflammation, arterial calcification, aortic stenosis; heart disease; heart failure; aortic stenosis, atherosclerosis, pathological angiogenesis, such as ocular angiogenesis or a disease or condition associated with ocular angiogenesis, e.g. neovascularization related to cancer; and eye diseases, such as age-related macular degeneration and corneal neovascularization; atherosclerosis; endocrine disorders, such as Addison, autoimmune hypophysitis; metabolic diseases such as diabetes; type 2 diabetes; insulin resistance; obesity; Diastolic HF; atrophic diseases in the brain, such as Alzheimer's and Parkinson's, atrophic diseases in the cerebellum, such as cerebellar atrophy, atrophic spinal diseases such as ALS; disorders related to transplantation in organs, such as anti-rejection prophylaxis, anti-acute rejection, anti-chronic rejection; acute burn; acute inflammatory reaction; chronic acute skin graft rejection; chronic scarring; asthma and other interstitial lung diseases, including Hermansky-Pudlak syndrome, pulmonary arterial hypertension, Rheumatoid disease associated interstitial lung disease RA-ILD, Systemic Sclerosis SSc-ILD, lung disease with fibrosis such as COPD (Chronic Obstructive Pulmonary Disease) and asthma; Otosclerosis, mesothelioma; post-surgery disorders, such as anti-keloid, anti-stricture, anti-adhesion, anti-thrombosis, fibrosis/scar reduction following cosmetic procedures; toxin exposure disorders, such as toxic hepatitis, cholera toxin related, mushroom toxin based acute renal failure, pertussis toxin, Aeromonas hydrophila enterotoxin, cadmium induced cardiac toxicity, helicobacter O-antigen related toxicity, LPS based toxicity, Streptozotocin toxicity, asbestos exposure, Nephrogenic Systemic Fibrosis (Post Contrast Agents); Tissue injury, such as Spinal cord injury, Peripheral nerve repair; congenital hepatic fibrosis; hereditary fibrosing poikiloderma with tendon contractures, myopathy, and pulmonary fibrosis; liver disorders, such as non-alcoholic steatohepatitis (NASH) or non-alcoholic fatty liver disease, liver cirrhosis of various origins, such as alcoholic and non-alcoholic, autoimmune cirrhosis such as primary biliary cirrhosis and sclerosing cholangitis, virally induced cirrhosis, cirrhosis induced by genetic disease; Liver cancer, cholangiocarcinoma, biliary tract cancer; neurodegenerative disorders such as Parkinsons disease, Alzheimers disease, cognitive impairment, cerebrovascular diseases such as stroke, traumatic brain injury, Huntington's disease, amyotrophic lateral sclerosis (ALS), multiple sclerosis (MS), peripheral nephropathy, in a mammal, such as a human.

Another aspect of the present invention concerns combination therapy involving administering a compound of formula (I) of the present invention together with a therapeutically active compound different from the compound of formula (I) (interchangeable with “a different therapeutically active compound”). In one embodiment the present invention relates to a combination of a compound of formula (I) and a different therapeutically active compound for use in treatment of a disorder relating to the binding of a galectin-⅓ to a ligand in a mammal. Such disorders are disclosed below.

In an embodiment of the present invention, a therapeutically effective amount of at least one compound of formula (I) of the present invention is administered to a mammal in need thereof in combination with a different therapeutically active compound. In a further embodiment, said combination of a compound of formula (I) together with a different therapeutically active compound is administered to a mammal suffering from a disorder selected from the group consisting of inflammation; fibrosis, such as pulmonary fibrosis, liver fibrosis, kidney fibrosis, ophthalmological fibrosis and fibrosis of the skin and heart; scarring; keloid formation; aberrant scar formation; surgical adhesions; septic shock; cancer, such as carcinomas, sarcomas, leukemias and lymphomas, such as T-cell lymphomas; metastasising cancers; autoimmune diseases, such as psoriasis, rheumatoid arthritis, Crohn's disease, ulcerative colitis, ankylosing spondylitis, systemic lupus erythematosus; metabolic disorders; heart disease; heart failure; pathological angiogenesis, such as ocular angiogenesis or a disease or condition associated with ocular angiogenesis, e.g. neovascularization related to cancer; and eye diseases, such as age-related macular degeneration and corneal neovascularization; atherosclerosis; metabolic diseases such as diabetes; type 2 diabetes; insulin resistens; obesity; Diastolic HF; asthma and other interstitial lung diseases, including Hermansky-Pudlak syndrome, mesothelioma; liver disorders, such as non-alcoholic steatohepatitis or non-alcoholic fatty liver disease.

A non-limiting group of cancers given as examples of cancers, including both solid and liquid cancers, that may be treated, managed and/or prevented by administration of a compound of formula (I) in combination with a different therapeutically active compound is selected from: colon carcinoma, breast cancer, head and neck cancer, testis cancer, urothelial cancer, pancreatic cancer, ovarian cancer, prostate cancer, fibrosarcoma, myxosarcoma, liposarcoma, chondrosarcoma, osteogenic sarcoma, chordoma, angiosarcoma, endotheliosarcoma, lymphangeosarcoma, lymphangeoendothelia sarcoma, synovioma, mesothelioma, Ewing's sarcoma, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystandeocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioblastomas, neuronomas, craniopharingiomas, schwannomas, glioma, astrocytoma, medulloblastoma, craniopharyngioma, ependymoma, pinealoma, hemangioblastoma, acoustic neuroama, oligodendroglioma, meningioma, melanoma, neuroblastoma, retinoblastoma, leukemias and lymphomas, acute lymphocytic leukemia and acute myelocytic polycythemia vera, multiple myeloma, Waldenstrom's macroglobulinemia, and heavy chain disease, acute nonlymphocytic leukemias, chronic lymphocytic leukemia, chronic myelogenous leukemia, Hodgkin's Disease, non-Hodgkin's lymphomas, rectum cancer, urinary cancers, uterine cancers, oral cancers, skin cancers, stomach cancer, brain tumors, liver cancer, laryngeal cancer, esophageal cancer, mammary tumors, childhood-null acute lymphoid leukemia (ALL), thymic ALL, B-cell ALL, acute myeloid leukemia, myelomonocytoid leukemia, acute megakaryocytoid leukemia, Burkitt's lymphoma, acute myeloid leukemia, chronic myeloid leukemia, and T cell leukemia, small and large non-small cell lung carcinoma, acute granulocytic leukemia, germ cell tumors, endometrial cancer, gastric cancer, cancer of the head and neck, chronic lymphoid leukemia, hairy cell leukemia and thyroid cancer.

In some aspects of the present invention, the administration of at least one compound of formula (I) of the present invention and at least one additional therapeutic agent demonstrates therapeutic synergy. In some aspects of the methods of the present invention, a measurement of response to treatment observed after administering both at least one compound of formula (I) of the present invention and the additional therapeutic agent is improved over the same measurement of response to treatment observed after administering either the at least one compound of formula (I) of the present invention or the additional therapeutic agent alone.

A further aspect of the present invention concerns combination therapy involving administering a compound of formula (I) of the present invention together with an anti-fibrotic compound different from the compound of formula (I) to a mammal in need thereof. In a further embodiment, such anti-fibrotic compound may be selected from the following non-limiting group of anti-fibrotic compounds: pirfenidone, nintedanib, simtuzumab (GS-6624, AB0024), BG00011 (STX100), PRM-151, PRM-167, PEG-FGF21, BMS-986020, FG-3019, MN-001, IW001, SAR156597, GSK2126458, PAT-1251 and PBI-4050.

A further aspect of the present invention concerns combination therapy involving administering a compound of formula (I) of the present invention together with an anti-cardiovascular compound different from the compound of formula (I) to a mammal in need thereof.

A still further aspect of the present invention concerns combination therapy involving administering a compound of formula (I) in combination with a further conventional cancer treatment such as chemotherapy and/or radiotherapy, and/or treatment with immunostimulating substances, and/or gene therapy, and/or treatment with antibodies and/or treatment using dendritic cells, to a mammal in need thereof.

In an embodiment the compound of formula (I) is administered together with at least one additional therapeutic agent selected from an antineoplastic chemotherapy agent. In a further embodiment, the antineoplastic chemotherapeutic agent is selected from: all-trans retinoic acid, Actimide, Azacitidine, Azathioprine, Bleomycin, Carboplatin, Capecitabine, Cisplatin, Chlorambucil, Cyclophosphamide, Cytarabine, Daunorubicin, Docetaxel, Doxifluridine, Doxorubicin, Epirubicin, Etoposide, Fludarabine, Fluorouracil, Gemcitabine, Hydroxyurea, Idarubicin, Irinotecan, Lenalidomide, Leucovorin, Mechlorethamine, Melphalan, Mercaptopurine, Methotrexate, Mitoxantrone, Oxaliplatin, Paclitaxel, Pemetrexed, Revlimid, Temozolomide, Teniposide, Thioguanine, Valrubicin, Vinblastine, Vincristine, Vindesine and Vinorelbine. In one embodiment, a chemotherapeutic agent for use in the combination of the present agent may, itself, be a combination of different chemotherapeutic agents. Suitable combinations include FOLFOX and IFL. FOLFOX is a combination which includes 5-fluorouracil (5-FU), leucovorin, and oxaliplatin. IFL treatment includes irinotecan, 5-FU, and leucovorin.

In a further embodiment of the present invention, the further conventional cancer treatment includes radiation therapy. In some embodiments, radiation therapy includes localized radiation therapy delivered to the tumor. In some embodiments, radiation therapy includes total body irradiation.

In other embodiments of the present invention the further cancer treatment is selected from the group of immunostimulating substances e.g. cytokines and antibodies. Such cytokines may be selected from the group consisting of, but not limited to: GM-CSF, type I IFN, interleukin 21, interleukin 2, interleukin 12 and interleukin 15. The antibody is preferably an immunostimulating antibody such as anti-CD40 or anti-CTLA-4 antibodies. The immunostimulatory substance may also be a substance capable of depletion of immune inhibitory cells (e.g. regulatory T-cells) or factors, said substance may for example be E3 ubiquitin ligases. E3 ubiquitin ligases (the HECT, RING and U-box proteins) have emerged as key molecular regulators of immune cell function, and each may be involved in the regulation of immune responses during infection by targeting specific inhibitory molecules for proteolytic destruction. Several HECT and RING E3 proteins have now also been linked to the induction and maintenance of immune self-tolerance: c-Cbl, Cbl-b, GRAIL, Itch and Nedd4 each negatively regulate T cell growth factor production and proliferation.

In some embodiments of the present invention the compound of formula (I) is administered together with at least one additional therapeutic agent selected from a checkpoint inhibitor. In some embodiments of the invention, the checkpoint inhibitor is acting on one or more of the following, non-limiting group of targets: CEACAM1, galectin-9, TIM3, CD80, CTLA4, PD-1, PD-L1, HVEM, BTLA, CD160, VISTA, B7-H4, B7-2, CD155, CD226, TIGIT, CD96, LAG3, GITF, OX40, CD137, CD40, IDO, and TDO. These are known targets and some of these targets are described in Melero et al., Nature Reviews Cancer (2015). Examples of check point inhibitors administered together with the compound of formula (1) are Anti-PD-1: Nivolumab, Pembrolizumab, Cemiplimab. Anti-PD-L1: Atezolizumab, Avelumab, Durvalumab and one Anti-CTLA-4: Ipilimumab. Each one of these check point inhibitors can be made the subject of an embodiment in combination with any one of the compounds of formula (1).

In some embodiments of the present invention the compound of formula (I) is administered together with at least one additional therapeutic agent selected from an inhibitor of indoleamine-2,3-dioxygenase (IDO).

In some embodiments of the present invention the compound of formula (I) is administered together with at least one additional therapeutic agent selected from one or more inhibitors of the CTLA4 pathway. In some embodiments, the inhibitor of the CTLA4 pathway is selected from one or more antibodies against CTLA4.

In some embodiments of the present invention the compound of formula (I) is administered together with at least one additional therapeutic agent selected from one or more inhibitors of the PD-1/PD-L pathway. In some embodiments, the one or more inhibitors of the PD-1/PD-L pathway are selected from one or more antibodies or antibody fragments against PD-1, PD-L1, and/or PD-L2, or other ways by which an anti-PD1 antibodies can be induced such as mRNA based introduction of genetic material which sets forth in-body production of anti-PD1 or anti-PDL1 antibodies or fragments of such antibodies.

In a still further aspect the present invention relates to a process of preparing a compound of formula II or a pharmaceutically acceptable salt or solvate thereof comprising the step a1 where A¹, B¹ and R¹ are defined as above under formula 1;

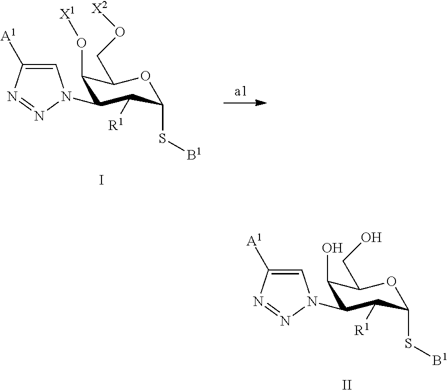

a1) Reacting a compound of formula I wherein X¹ and X² together form a protective group such as benzylidene in the presence of an acid, such as TFA, in an inert organic solvent, such as DCM, followed by neutralisation with an base, such as triethylamine, optionally at temperatures below room temperature, to give a compound of formula II; optionally reacting a compound of formula I wherein X¹ and X² are two protective groups, such as acetates, in the presence of a base, such as triethylamine, sodium hydroxide or sodium methoxide in an organic solvent, such as methanol, optionally in the presence of water followed by neutralization using an acid, such as HCl, to give a compound to formula II.

In a still further aspect the present invention relates to a process of preparing a compound of formula II or a pharmaceutically acceptable salt or solvate thereof comprising the step a2 where A¹ and B¹ are defined as above under formula 1;

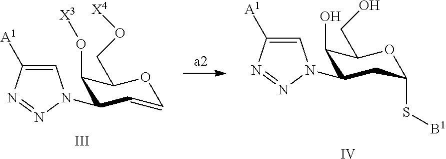

a2) Reacting a compound of formula III, wherein X³ and X⁴ are hydrogens or protective groups, such as acetates, with a compound of formula B¹—SH in an organic solvent, such as toluene, optionally in the presence of a catalyst such as oxotrichloro[(dimethylsulfide)triphenylphosphine oxide]rhenium(V) or BF₃OEt₂, optionally at elevated temperature to give a compound of formula IV; when X³ and X⁴ are protective groups, such as acetates, these could be removed in an additional step in the presence of base, such as triethylamine, LiOH or sodium methoxide in a suitable solvent, such as methanol and water, to give a compound of formula IV.

In a still further aspect the present invention relates to a process of preparing a compound of formula II or a pharmaceutically acceptable salt or solvate thereof comprising the step a3 where A¹, B¹ and R¹ are defined as above under formula 1;

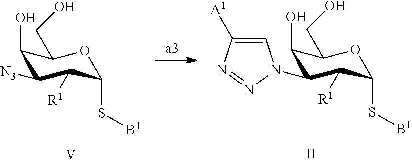

a3) Reacting the compound of formula V with a compound of formula A¹-CC-H or A¹-CC-TMS or A¹-CC-TIPS in an inert solvent, such as DMF or acetonitrile, using a base, such as diisopropylethylamine or L-ascorbic acid sodium salt, catalyzed by a cupper salt such as CuI or copper(II) sulfate, optionally using a reagent such as CsF or TBAF to provide a compound of the formula II; optionally a compound of the formula

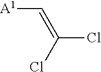

wherein A¹ is defined as above under formula 1 is reacted with butyllithium in an inert solvent, such as tetrahydrofuran, at temperatures between −78 and −30° C. followed by neutralization using an acid, such as acetic acid to give an intermediate which is further reacted with a compound of the formula V defined as above in an inert solvent, such as tetrahydrofuran, using a base, such as triethylamine, catalyzed by a cupper salt such as CuI optionally at elevated temperature to provide a compound of the formula II.

In a still further aspect, the present invention relates to a process of preparing a compound of formula VIII or a pharmaceutically acceptable salt or solvate thereof comprising the steps a4-a5 where A¹ and B¹ are defined as above under formula 1;

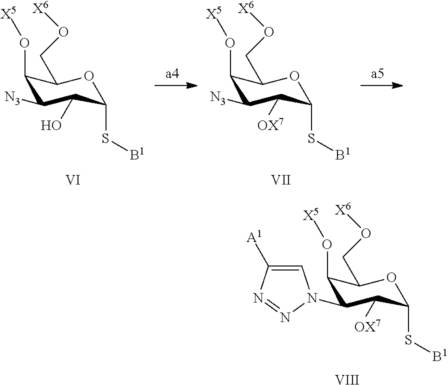

a4) Reacting a compound of formula VI wherein X⁵ and X⁶ (X⁵-X⁶) together form a protecting group such as a bezylidene with a compound of formula X⁷-L¹, where X⁷ taken together with O is OX⁷ which is selected from c) under the definition of R¹ above under formula 1, and L¹ is defined as a leaving groups such as a halide, such as Cl, Br, I or a sulfate ester such as a mesylate, tosylate or triflate in an organic solvent such as DMF, optionally in the presence of a reagent such as NaH, Cs₂CO₃ or AgO, to give a compound of the formula VII.
a5) Reacting the compound of formula VII wherein X⁵-X⁶ together form a protecting group such as a bezylidene with a compound of formula A¹-CC-H or A¹-CC-TMS or A¹-CC-TIPS in an inert solvent, such as DMF or acetonitrile, using a base, such as diisopropylethylamine or L-ascorbic acid sodium salt, catalyzed by a cupper salt such as CuI or copper(II) sulfate, optionally using a reagent such as CsF or TBAF, optionally at elevated temperature to provide a compound of the formula VIII; optionally reacting the compound of formula VII wherein X⁵-X⁶ are protective groups such as acetates with a compound of formula A¹-CC-H or A¹-CC-TMS or A¹-CC-TIPS in an inert solvent, such as DMF or acetonitrile, using a base, such as diisopropylethylamine or L-ascorbic acid sodium salt, catalyzed by a cupper salt such as CuI or copper(II) sulfate, optionally using a reagent such as CsF or TBAF, optionally at elevated temperature to provide a compound of the formula VIII wherein X⁵-X⁶ are protective groups such as acetates.

In a still further aspect, the present invention relates to a process of preparing a compound of formula X or a pharmaceutically acceptable salt or solvate thereof comprising the step a6 where A¹ and R¹ are defined as above under formula 1;

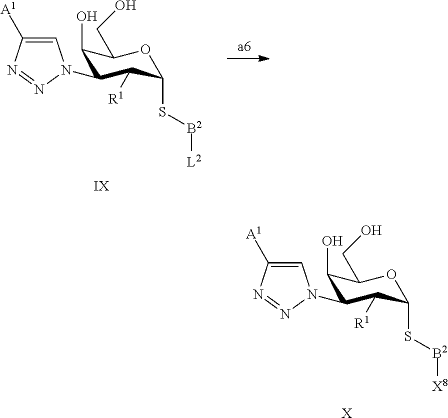

a6) Reacting a compound of formula IX wherein B² is selected from B¹ section b) and d) under formula 1 and L² is defined as a halide such as I, Br or Cl, with a metallorganic compound such Zn(CN)₂ in the presence of Zn and 1,1′-bis(diphenylphosphino)ferrocene and Pd₂(dba)₃ in a suitable organic solvent such as DMF optionally at elevated temperatures to give a compound X wherein X⁸ is defined as —CN; optionally a compound of formula IX defined as above is reacted with a borinane such as 2,4,6-trimethyl-1,3,5,2,4,6-trioxatriborinane in the prescence of a metalloorganic catalys such as Pd(PPh₄)₃ and an inorganic base such as K₂CO₃ in a suitable solvent such as 1,4-dioxane optionally at elevated temperatures to give a compound of formula X wherein X⁸ is defined as methyl; optionally a compound of formula IX defined as above is reacted with a stannane such as tributyl(oxazol-2-yl)stannane or tributyl(thiazole-2-yl)stannane in the presence of an metalloorganic catalyst such as Pd(PPh₃)₄, optionally in the presence CsF, optionally at elevated temperatures to give a compound of formula X wherein X⁸ is a five or six membered heteroaromatic ring optionally substituted; optionally a compound of formula IX defined as above is reacted with a heterocyclic borinate, such as tert-butyl 4-(4,4,5,5-tetramethyl-1,3,2-dioxaborolan-2-yl)-3,6-dihydro-2H-pyridine-1-carboxylate in the presence of a catalyst such as bis(triphenylphosphine)palladium(II) chloride optionally in the presence of base such as potassium carbonate, optionally in the presence of water in an organic solvent such as 1,4 dioxane, optionally at elevated temperatures.to give a compound of formula X wherein X⁸ is defined as a five or six membered heteroaromatic ring or a heterocyclic ring.; optionally a compound of formula IX could be reacted with an alkyne or protected alkyne such as ethynyl(trimethyl)silane in the presence of one or more metallorganic reagents such as CuI, bis(triphenylphosphine)palladium (II) chloride in an inert solvent such as THF, optionally in the presence of an organic base such as triethylamine or DIPEA, optionally at elevated temperatures such as 30-80° C. If the alkyne reagent was protected with a silyl protective group such as trimethylsilane the protective group could be removed by addition of a reagent such as TBAF or KF in a consecutive step.

In a still further aspect the present invention relates to a process of preparing a compound of formula XII where A¹ and R¹ are defined as above under formula 1 and B³ is selected from B1 section b) and d) under the compound of formula 1 wherein X¹⁰ is defined as —CONR⁶R⁷ or —CONR¹²R¹³ wherein R⁶, R⁷, R¹² and R¹³ are defined as for the compound of formula 1, methyl, heterocycle, —CN, ethynyl, spiroheterocycle, CONH₂, COOH, —SCH₃, —COOCH₃ comprising the step a7;

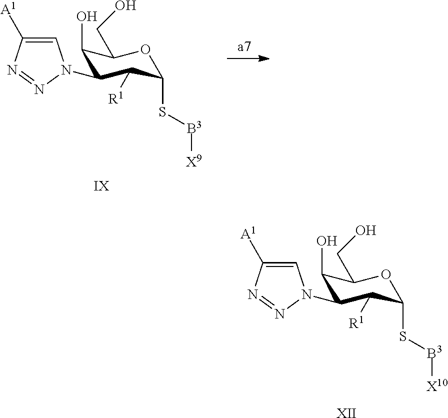

a7) Reacting a compound of formula XI wherein X⁹ is defined as —COOH with an amine reagent such as HNR⁶R⁷ or HNR¹²R¹³ in the presence of an amide coupling reagent such as HATU optionally in the presence of an organic base such as 4-methylmorpholine optionally in the presence of a reagent such as methansulfonic acid in an inert solvent such as DMF to give a compound formula XII wherein X¹⁰ is defined as —CONR⁶R⁷ or CONR¹²R¹³; optionally reacting a compound of formula XI wherein X⁹ is a halide such as I, Br and Cl with a heterocyclic borinane such as 2,4,6-trimethyl-1,3,5,2,4,6-trioxatriborinane, in the presence of Pd(PPh₄)₃, K₂CO₃ in an inert solvent such as dioxane optionally at elevated temperature and optionally under an inert atmosphere to give a compound formula XII wherein X¹⁰ is defined as a methyl; optionally reacting a compound of formula XI wherein X⁹ is a halide such as I, Br and Cl with a heterocyclic dioxaborolane such as 5-(4,4,5,5-tetramethyl-1,3,2-dioxaborolan-2-yl)pyrimidine, 4-(4,4,5,5-tetramethyl-1,3,2-dioxaborolan-2-yl)pyridine, tert-butyl 4-(4,4,5,5-tetramethyl-1,3,2-dioxaborolan-2-yl)-3,6-dihydro-2H-pyridine-1-carboxylate in an inert solvent such as 1,4-dioxane/water using a metalloorganic reagent such as bis(triphenylphosphine)palladium(II) chloride and a base such as K₂CO₃, optionally heating to 100° C. for 1 h in a microwave reactor to give a compound formula XII wherein X¹⁰ is defined as an heterocycle; optionally reacting a compound of formula XI wherein X⁹ is a halide such as I, Br and Cl with an heterocyclic boronic acid such as 3-pyridylboronic acid in an inert solvent such as DMF using a metalloorganic reagent such as bis(triphenylphosphine)palladium(II) chloride and a base such as K₂CO₃ at room temperature to give a compound formula XII wherein X¹⁰ is defined as an heterocycle; optionally reacting a compound of formula XI wherein X⁹ is a halide such as I, Br and Cl with a heterocyclic stannane such as tributyl-(2-pyridyl)stannane, tributyl(oxazol-2-yl)stannane, tributyl(thiazole-2-yl)stannane in an inert solvent such as DMF using a metalloorganic reagent such as bis(triphenylphosphine)palladium(II) chloride or palladium tetrakis optionally with CsF at room temperature or elevated temperatures to give a compound formula XII wherein X¹⁰ is defined as an heterocycle; optionally reacting a compound of formula XI wherein X⁹ is a halide such as I, Br and Cl with a metallocyanoreagent such as Zn(CN)₂ in an inert solvent such as DMF using a metalloorganic reagent such as Pd₂(dibenzylideneacetone) and zinc at elevated temperatures to give a compound formula XII wherein X¹⁰ is defined as a —CN; optionally reacting a compound of formula XI wherein X⁹ is a halide such as I, Br and Cl with ethynyl or TMS-ethynyl in the presence of metallorganic reagents such as bis(triphenylphosphine)palladium (II) chloride and CuI in the presence of an organic base in an inert solvent as DMF optionally at elevated temperature to give a compound formula XII wherein X¹⁰ is defined as an ethynyl; optionally reacting a compound of formula XI wherein X⁹ is a halide such as I, Br and Cl with a heterocycle or spiro heterocycle such as a spiro heterocycle, such as N-(2-oxa)-6-azaspiro[3.3]heptanyl using an organic base such as DIPEA in an inert solvent such as DMF at elevated temperature such as 130° C. in a microwave reactor to give a compound formula XII wherein X¹⁰ is defined as an heterocycle or a spirocycle; optionally reacting a compound of formula XI wherein X⁹ is a cyanogroup with a base such as sodium hydroxide at elevated temperature in a solvent such as ethanol and water to give a compound of formula XII wherein X¹⁰ is —COOH; optionally reacting a compound of formula XI wherein X⁹ is defined as —COOX¹¹ and X¹¹ is defined as an aryl or a straight or branched c1-c5 alkyl optionally substituted with an aryl, with a base such as lithium hydroxide or sodium hydroxide at elevated temperature in water optionally mixed with another organic solvent such as ethanol or acetonitrile to give a compound of formula XII wherein X¹⁰ is defined as —COOH; optionally reacting a compound of formula XI wherein X⁹ is a halide such as I, Br and Cl with an alkyl thiol nucleophile such as sodium thiomethoxide in a solvent such as DMF to give a compound of formula XII wherein X¹⁰ is —SCH₃; optionally reacting a compound of formula XI wherein X⁹ is defined as —COOH with an alkyl halide such as methyliodide in a solvent such as DMF in the presence of a base such as Cs₂CO₃ to give a compound of formula XII wherein X¹⁰ is —COOCH₃.

In a still further aspect, the present invention relates to a process of preparing a compound of formula III or a pharmaceutically acceptable salt or solvate thereof comprising the steps a8-a9 where A¹ is defined as above under formula 1 and X³ and X⁴ are optionally and independently selected from hydrogen and acetate;

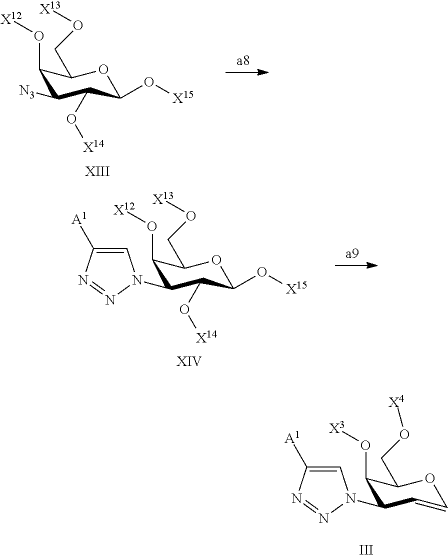

a8) Reacting the compound of formula XIII wherein X¹²-X¹⁵ are protecting groups such as acetates with a compound of formula A¹-CC-H or A¹-CC-TMS or A¹-CC-TIPS in an inert solvent, such as DMF or acetonitrile, using a base, such as DIPEA or L-ascorbic acid sodium salt, catalyzed by a cupper salt such as CuI or copper(II) sulfate, optionally using a reagent such as CsF or TBAF, optionally at elevated temperature to provide a compound of the formula XIV.
a9) Reacting a compound of formula XIV with an acid such as HBr or acetic acid in an inert solvent such as DCM over 0-10 h after which the product is isolated and reacted further in the presence of ammonium chloride and zinc in a solvent such as acetonitrile over 3-7 days to give a compound of formula III wherein X³—X⁴ are protective groups such as acetates.

In a still further aspect, the present invention relates to a process of preparing a compound of formula XIX or a pharmaceutically acceptable salt or solvate thereof comprising the steps a10-a13 where B¹ is defined as above under formula 1;

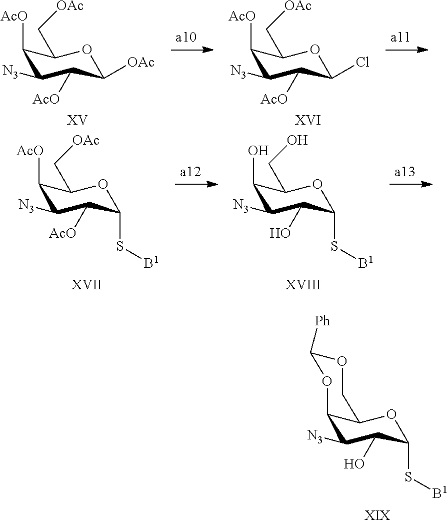

a10) Reacting a compound of the formula XV with a chlorinating reagent such as dichloromethylmethylether or PCl₅ in the presence of a lewis acid such as BF₃ Et₂O in an inert solvent such as DCM to give a compound of formula XVI.
a11) Reacting a compound of the formula XVI with a nucleophile like a compound of formula HS-B¹ in the presence of a base such as sodium hydride in an inert solvent such as DMF to give a compound of formula XVII.
a12) Reacting a compound of formula XVII in the presence of a base, such as triethylamine, sodium hydroxide or sodium methoxide in an organic solvent, such as methanol, optionally in the presence of water followed by neutralization using an acid, such as HCl, to give a compound to formula XVIII.
a13) Reacting a compound of formula XVIII with a reagent such as benzaldehyde dimethyl acetal in the prescence of an acid such as D(+)-10-Camphorsulfonic acid, in an inert solvent such as acetonitrile or DMF, optionally at elevated temperature and optionally at reduced pressure distilling off methanol to give a compound of formula XIX.

In a still further aspect the present invention relates to a process of preparing a compound of formula XXI where R¹ is defined as above under formula 1 and B³ is selected from B¹ section b) and d) under the compound of formula 1 wherein X¹⁷ is defined as —CONR⁶R⁷ or —CONR¹²R¹³ wherein R⁶, R⁷, R¹² and R¹³ are defined as for the compound of formula 1, methyl, heterocycle, —CN, ethynyl, spiroheterocycle, CONH₂, COOH, —SCH₃, —COOCH₃ comprising the step a14;

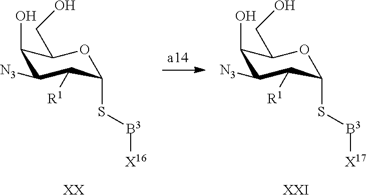

a14) Reacting a compound of formula XX wherein X¹⁶ is defined as —COOH with an amine reagent such as HNR⁶R⁷ or HNR¹²R¹³ in the presence of an amide coupling reagent such as HATU optionally in the presence of an organic base such as 4-methylmorpholine optionally in the presence of a reagent such as methansulfonic acid in an inert solvent such as DMF to give a compound formula XXI wherein X¹⁷ is defined as —CONR⁶R⁷ or CONR¹²R¹³; optionally reacting a compound of formula XX wherein X¹⁶ is a halide such as I, Br and Cl with a heterocyclic borinane such as 2,4,6-trimethyl-1,3,5,2,4,6-trioxatriborinane, in the presence of Pd(PPh₄)₃, K₂CO₃ in an inert solvent such as dioxane optionally at elevated temperature and optionally under an inert atmosphere to give a compound formula XXI wherein X¹⁷ is defined as a methyl; optionally reacting a compound of formula XX wherein X¹⁶ is a halide such as I, Br and Cl with a heterocyclic dioxaborolane such as 5-(4,4,5,5-tetramethyl-1,3,2-dioxaborolan-2-yl)pyrimidine, 4-(4,4,5,5-tetramethyl-1,3,2-dioxaborolan-2-yl)pyridine, tert-butyl 4-(4,4,5,5-tetramethyl-1,3,2-dioxaborolan-2-yl)-3,6-dihydro-2H-pyridine-1-carboxylate in an inert solvent such as 1,4-dioxane/water using a metalloorganic reagent such as bis(triphenylphosphine)palladium(II) chloride and a base such as K₂CO₃, optionally heating to 100° C. for 1 h in a microwave reactor to give a compound formula XXI wherein X¹⁷ is defined as an heterocycle; optionally reacting a compound of formula XX wherein X¹⁶ is a halide such as I, Br and Cl with an heterocyclic boronic acid such as 3-pyridylboronic acid in an inert solvent such as DMF using a metalloorganic reagent such as bis(triphenylphosphine)palladium(II) chloride and a base such as K₂CO₃ at room temperature to give a compound formula XXI wherein X¹⁷ is defined as an heterocycle; optionally reacting a compound of formula XX wherein X¹⁶ is a halide such as I, Br and Cl with a heterocyclic stannane such as tributyl-(2-pyridyl)stannane, tributyl(oxazol-2-yl)stannane, tributyl(thiazole-2-yl)stannane in an inert solvent such as DMF using a metalloorganic reagent such as bis(triphenylphosphine)palladium(II) chloride or palladium tetrakis optionally with CsF at room temperature or elevated temperatures to give a compound formula XXI wherein X¹⁷ is defined as an heterocycle; optionally reacting a compound of formula XX wherein X¹⁶ is a halide such as I, Br and Cl with a metallocyanoreagent such as Zn(CN)₂ in an inert solvent such as DMF using a metalloorganic reagent such as Pd₂(dibenzylideneacetone) and zinc at elevated temperatures to give a compound formula XXI wherein X¹⁷ is defined as a —CN; optionally reacting a compound of formula XX wherein X¹⁶ is a halide such as I, Br and Cl with ethynyl or TMS-ethynyl in the presence of metallorganic reagents such as bis(triphenylphosphine)palladium (II) chloride and CuI in the presence of an organic base in an inert solvent as DMF optionally at elevated temperature to give a compound formula XXI wherein X¹⁷ is defined as an ethynyl; optionally reacting a compound of formula XX wherein X¹⁶ is a halide such as I, Br and Cl with a heterocycle or spiro heterocycle such as a spiro heterocycle, such as N-(2-oxa)-6-azaspiro[3.3]heptanyl using an organic base such as DIPEA in an inert solvent such as DMF at elevated temperature such as 130° C. in a microwave reactor to give a compound formula XXI wherein X¹⁷ is defined as an heterocycle or a spirocycle; optionally reacting a compound of formula XX wherein X¹⁶ is a cyanogroup with a base such as sodium hydroxide at elevated temperature in a solvent such as ethanol and water to give a compound of formula XXI wherein X¹⁷ is —COOH; optionally reacting a compound of formula XX wherein X¹⁶ is defined as —COOX¹⁸ and X¹⁸ is defined as an aryl or a straight or branched c1-c5 alkyl optionally substituted with an aryl, with a base such as lithium hydroxide or sodium hydroxide at elevated temperature in water optionally mixed with another organic solvent such as ethanol or acetonitrile to give a compound of formula XXI wherein X¹⁷ is defined as —COOH; optionally reacting a compound of formula XX wherein X¹⁶ is a halide such as I, Br and Cl with an alkyl thiol nucleophile such as sodium thiomethoxide in a solvent such as DMF to give a compound of formula XXI wherein X¹⁷ is —SCH₃; optionally reacting a compound of formula XX wherein X¹⁶ is defined as —COOH with an alkyl halide such as methyliodide in a solvent such as DMF in the presence of a base such as Cs₂CO₃ to give a compound of formula XXI wherein X¹⁷ is —COOCH₃.

In a still further aspect the present invention relates to a process of preparing a compound of the formula XXIII comprising the step a15, wherein B¹ is defined as above under formula 1;

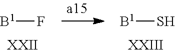

a15) Reacting a compound of the formula XXII with Na₂S·10H₂O in the presence of a base such as NaOH in an inert solvent such as DMF to give a compound of formula XXIII.

In a still further aspect the present invention relates to a process of preparing a compound of formula XXVI comprising the steps a16-a17, wherein B¹ is defined as above under formula 1;

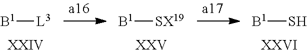

a16) Reacting a compound of the formula XXIV wherein L³ is a leaving group such as bromine or iodine with a compound of the formula X¹⁹—SH wherein X¹⁹ is a protective group such as a benzyl group in the presence of a base such as DIPEA in an inert solvent such as dioxane at elevated temperature to give a compound of formula XXV; optionally reacting a compound of formula XXIV wherein L³ is a leaving group such as bromine or iodine with (2,4-dimethoxyphenyl)methanethiol in the presence of a metalloorganic ligand such as bis(dibenzylideneacetone)palladium optionally in the presence of a ligand such as 4,5-bis(diphenylphosphino)-9,9-dimethylxanthene in an inert solvent such as dioxane at elevated temperature to give a compound of the formula XXV.
a17) Reacting a compound of the formula XXV with AlCl₃ in a solvent such as toluene to give a compound of the formula XXVI; optionally reacting a compound of the formula XXV in the presence of TFA and triethyl silane to give a compound of formula XXVI.

In a still further aspect the present invention relates to a process of preparing a compound of formula XXIX comprising the step a18 wherein R², R³ and R⁴ are defined as above under formula 1;

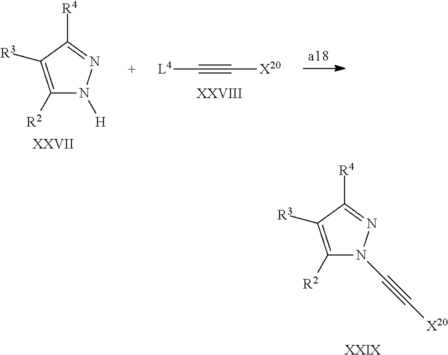

a18) Reacting a compound of formula XXVII with a compound of formula XXVIII wherein L⁴ is defined as a halide such as bromine or iodine and X²⁰ is either a hydrogen or a protective group such as triisopropylsilane in the presence of copper iodide and a base such as Cs₂CO₃ in an inert solvent, such as 1,4 dixane and PEG400, to give a compound of the formula XXIX; optionally reacting a compound of formula XXVII with a compound of formula XXVIII wherein L⁴ is defined as a hydrogen and X²⁰ is a protective group such as triisopropylsilane in the presence of a catalyst, such as copper(II) acetate, and a base such as Na₂CO₃ in an inert solvent, such as toluene, at elevated temperature to give a compound of the formula XXIX.

In a still further aspect the present invention relates to a process of preparing a compound of formula XXXIII comprising the steps a19-a21 wherein R², R³ and R⁴ are defined as above under formula 1:

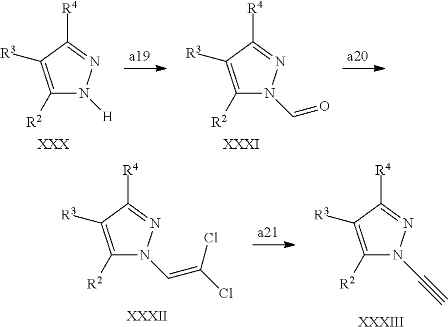

a19) Reacting a compound of formula XXX with acetic anhydride in a solvent such as formic acid to give a compound of the formula XXXI.
a20) Reacting a compound of formula XXXI with carbon tetrachloride in the presence of triphenylphosphine in an inert solvent, such as tetrahydrofuran, at elevated temperature to give a compound of the formula XXXII.
a21) Reacting a compound of formula XXXII with butyllithium in an inert solvent, such as tetrahydrofuran, at temperatures between −78 and −30° C. followed by neutralization using an acid, such as acetic acid to give a compound of the formula XXXIII.

In a still further aspect the present invention relates to a process of preparing a compound of formula XXXVI comprising the steps a22-a23;

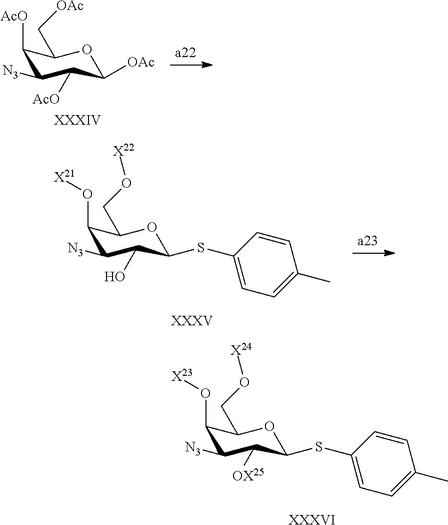

a22) Reacting a compound of the formula XXXIV with 4-methylbenzenethiol in the presence of boron trifluoride in an inert solvent, such as DCM, to give an intermediate which is deprotected in the presence of a base such as sodium methoxide in a solvent such as methanol to give a compound which is further reacted with a reagent such as benzaldehyde dimethyl acetal in the presence of an acid such as p-toluenesulphonic acid to give a compound of the formula XXXV wherein X²¹ and X²² together form a protecting group such as a benzylidene.
a23) Reacting a compound of the formula XXXV with a compound of formula X²⁵-L⁵, where X²⁵ taken together with O is OX²⁵ which is selected from c) under the definition of R¹ above under formula 1, and L⁵ is defined as a leaving groups such as a halide, such as Cl, Br, I or a sulfate ester such as a mesylate, tosylate or triflate in an organic solvent such as DMF, optionally in the presence of a reagent such as NaH, Cs₂CO₃ or AgO, to give a compound which is treated with an acid, such as TFA, in the presence of water to give a compound which is further reacted with acetic anhydride in pyridine to give a compound of formula XXXVI wherein X²³ and X³⁴ are acetates.

In a still further aspect the present invention relates to a process of preparing a compound of formula XXXX comprising the step a24-a26 where R¹ and B are defined as above under formula 1 and X²⁶ and X²⁷ are protective groups such as acetates, and X²⁸ is a protective group such as TIPS;

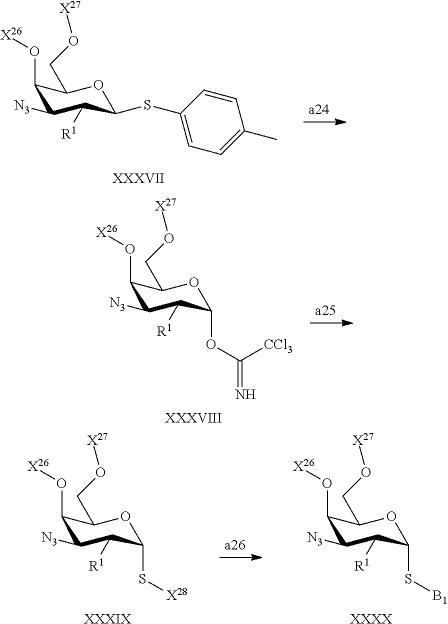

a24) Reacting a compound of the formula XXXVII with N-bromosuccinimide in a solvent mixture such as water and 1,4-dioxane to give a compound which is further reacted with trichloroacetonitrile in the presence of a base such as 1,8-diazabicyclo[5.4.0]undec-7-ene in an inert solvent such as DCM to give a compound of formula XXXVIII.
a25) Reacting a compound of the formula XXXVIII with a compound of the formula HS-X²⁸ in the presence boron trifluoride diethyl etherate in an inert solvent such as DCM to give a compound of formula XXXIX.
a26) Reacting a compound of the formula XXXIX with a compound of the formula B¹-L⁶, wherein L⁶ is defined as a leaving group such as I, Br, Cl and F, in the presence of a reagent, such as TBAF, in an inert solvent, such as acetonitrile, optionally at elevated temperature to give a compound of formula XXXX.

In the above reaction steps a1 to a26 whenever a diasteroisomeric compound is made it can be separated by chromatography such as using HPLC. Furthermore, in above process steps a1 to a26 an anomeric sulphur can be replaced by an O, SO, or SO₂ under similar reaction conditions to prepare analogs where S has been replaced.

### DETAILED DESCRIPTION OF THE INVENTION

The present compounds of formula (1) differ from prior art compounds particularly in that the pyranose ring is α-D-galactopyranose. It is important to emphasize that alpha and beta anomers are very different isomers and it is by no means considered to be obvious to the skilled person to expect same or similar activity of both anomers. Consequently, alpha and beta anomers do not in general posses the same activity, and this is common knowledge to the skilled person. The compounds of the present invention are novel α-D-galactopyranose compounds that unexpectedly have shown very high affinity and specificity for galectin-1 and are considered novel potent drug candidates. Some of the novel α-D-galactopyranose compounds have both galectin-1 and galectin-3 affinity and, as such have a broader disease treatment profile compared to selective galectin-1 inhibitors.

In broad aspect the present invention concerns a D-galactopyranose compound of formula (1)

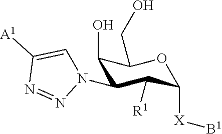

wherein
the pyranose ring is α-D-galactopyranose, and A¹, R¹, X and B¹ are as defined above.

In a further embodiment the compound of formula (1) R² is selected from the group consisting of H; halogen; OH; CN; SH; S—C₁₋₆ alkyl; C₁₋₆ alkyl, optionally substituted with a F; cyclopropyl, optionally substituted with a F; O-cyclopropyl optionally substituted with a F; OC₁₋₆ alkyl optionally substituted with a F; NR²⁴R²⁵, wherein R²⁴ is selected from H and C₁₋₆ alkyl, and R²⁵ is selected from H, C₁₋₃ alkyl, and C(═O)R²⁶, wherein R²⁶ is selected from H, and C₁₋₆ alkyl; C(═O)NR^{24a}R^{25a}, wherein R^{24a} is selected from H and C₁₋₆ alkyl, and R^{25a} is selected from H, C₁₋₃ alkyl, and C(═O)R^{26a}, wherein R^{26a} is selected from H, and C₁₋₆ alkyl; C(═O)OR^{24b}R^{25b}, wherein R^{24b} is selected from H and C₁₋₆ alkyl, and R^{25b} is selected from H, C₁₋₃ alkyl, and C(═O)R^{26b}, wherein R^{26b} is selected from H, and C₁₋₆ alkyl. In a still further embodiment R² is selected from H, halogen, methyl, amino, OH, and CN.

In a further embodiment the compound of formula (1) R³ is selected from the group consisting of H; halogen; OH; CN; SH; S—C₁₋₆ alkyl; C₁₋₆ alkyl, optionally substituted with a F; cyclopropyl, optionally substituted with a F; O-cyclopropyl optionally substituted with a F; OC₁₋₆ alkyl optionally substituted with a F; NR²⁴R²⁵, wherein R²⁴ is selected from H and C₁₋₆ alkyl, and R²⁵ is selected from H, C₁₋₃ alkyl, and C(═O)R²⁶, wherein R²⁶ is selected from H, and C₁₋₆ alkyl; C(═O)NR^{24a}R^{25a}, wherein R^{24a} is selected from H and C₁₋₆ alkyl, and R^{25a} is selected from H, C₁₋₃ alkyl, and C(═O)R^{26a}, wherein R^{26a} is selected from H, and C₁₋₆ alkyl; C(═O)OR^{24b}R^{25b}, wherein R^{24b} is selected from H and C₁₋₆ alkyl, and R^{25b} is selected from H, C₁₋₃ alkyl, and C(═O)R^{26b}, wherein R^{26b} is selected from H, and C₁₋₆ alkyl. In a still further embodiment R³ is selected from H, halogen, methyl, amino, OH, and CN.

In a further embodiment the compound of formula (1) R⁴ is selected from the group consisting of H; halogen; OH; CN; SH; S—C₁₋₆ alkyl; C₁₋₆ alkyl, optionally substituted with a F; cyclopropyl, optionally substituted with a F; O-cyclopropyl optionally substituted with a F; OC₁₋₆ alkyl optionally substituted with a F; NR²⁴R²⁵, wherein R²⁴ is selected from H and C₁₋₆ alkyl, and R²⁵ is selected from H, C₁₋₃ alkyl, and C(═O)R²⁶, wherein R²⁶ is selected from H, and C₁₋₆ alkyl; C(═O)NR^{24a}R^{25a}, wherein R^{24a} is selected from H and C₁₋₆ alkyl, and R^{25a} is selected from H, C₁₋₃ alkyl, and C(═O)R^{26a}, wherein R^{26a} is selected from H, and C₁₋₆ alkyl; C(═O)OR^{24b}R^{25b}, wherein R^{24b} is selected from H and C₁₋₆ alkyl, and R^{25b} is selected from H, C₁₋₃ alkyl, and C(═O)R^{26b}, wherein R^{26b} is selected from H, and C₁₋₆ alkyl. In a still further embodiment R⁴ is selected from H, halogen, methyl, amino, OH, and CN.

In a further embodiment R² is hydrogen, R³ is hydrogen and R⁴ is a halogen, such as Cl or F.

In a still further embodiment X is S. In another embodiment X is Se. In a still other embodiment X is SO. In another embodiment X is SO₂. In a still other embodiment X is O.

In a further embodiment B1 is selected from a C₁₋₆ alkyl or branched C₃₋₆ alkyl substituted with a five or six membered heteroaromatic ring, optionally substituted with a substituent selected from CN, a halogen, methyl optionally substituted with a F, OCH₃ optionally substituted with a F, OCH₂CH₃ optionally substituted with a F, OH, and R^{4a}—CONH— wherein R^{4a} is selected from C₁₋₃ alkyl and cyclopropyl; or a C₁₋₆ alkyl substituted with a phenyl, optionally substituted with a substituent selected from CN, a halogen, methyl optionally substituted with a F, OCH₃ optionally substituted with a F, OCH₂CH₃ optionally substituted with a F, OH, and R^{5a}—CONH— wherein R^{5a} is selected from C₁₋₃ alkyl and cyclopropyl.

In a still further embodiment B1 is selected from an aryl, such as phenyl or naphthyl, optionally substituted with a group selected from a halogen; a spiro heterocycle, such as N-(2-oxa)-6-azaspiro[3.3]heptanyl; C₂-alkynyl; CN; —COOH; COOC₁₋₄ alkyl; —CONR⁶R⁷, wherein R⁶ and R⁷ are independently selected from H, C₁₋₃ alkyl, cyclopropyl, and iso-propyl, or R⁶ and R⁷ together with the nitrogen form a heterocycloalkyl; C₁₋₃ alkyl, optionally substituted with a F; cyclopropyl, optionally substituted with a F; isopropyl, optionally substituted with a F; SC₁₋₃ alkyl, optionally substituted with a F; OC₁₋₃ alkyl, optionally substituted with a F; O-cyclopropyl, optionally substituted with a F; O-isopropyl, optionally substituted with a F; NR⁸R⁹, wherein R⁸ and R⁹ are independently selected from H, C₁₋₃ alkyl and isopropyl; OH; and R¹⁰—CONH— wherein R¹⁰ is selected from C₁₋₃ alkyl and cyclopropyl; an aryl; and a heterocycle.

In a further embodiment B1 is selected from a C₅₋₇ cycloalkyl, optionally substituted with a substituent selected from a halogen, C₂-alkynyl, CN, methyl optionally substituted with a F, OCH₃ optionally substituted with a F, OCH₂CH₃ optionally substituted with a F, OH, and R¹¹—CONH— wherein R¹¹ is selected from C₁_3 alkyl and cyclopropyl.

In a still further embodiment B¹ is selected from a heterocycle, such as heteroaryl or heterocycloalkyl, optionally substituted with a group selected from a halogen; a spiro heterocycle, such as N-(2-oxa)-6-azaspiro[3.3]heptanyl; C₂-alkynyl; CN; —COOH; COOC₁₋₄ alkyl; —CONR¹²R¹³, wherein R¹² and R¹³ are independently selected from H, C₁₋₃ alkoxy, branched C₃₋₆ alkyl, C₁₋₆ alkyl optionally substituted with a F, bicyclopentanyl, CH₂-cyclopropyl, and CH₂-cyclobutyl, or R¹² and R¹³ together with the nitrogen form a heterocycloalkyl; C₁₋₃ alkyl, optionally substituted with a F; cyclopropyl, optionally substituted with a F; isopropyl, optionally substituted with a F; SC₁₋₃ alkyl, optionally substituted with a F; OC₁₋₃ alkyl, optionally substituted with a F; O-cyclopropyl, optionally substituted with a F; O-isopropyl, optionally substituted with a F; SC₁₋₃ alkyl, optionally substituted with a F; NR¹⁴R¹⁵, wherein R¹⁴ and R¹⁵ are independently selected from H, C₁₋₃ alkyl and isopropyl; OH; an aryl; a heterocycle; and R¹⁶—CONH— wherein R¹⁶ is selected from C₁₋₃ alkyl and cyclopropyl. Typically, B1 is selected from a pyridinyl substituted with one, two or three groups selected from a Cl; Br; F; ethynyl; N-(2-oxa)-6-azaspiro[3.3]heptanyl; CO-azetidinyl; CONHCH₃; CONHCH₂CH₃; CON(CH₃)₂; CN; methyl; SCH₃; SCF₃; CF₃; imidazole; pyridin; pyrimidin; oxazol; and thiazol; such as pyridinyl substituted with one or two selected from Cl, Br, CN, and CONHCH₃. In a further embodiment B1 is selected from a pyridinyl substituted with one or two groups selected from halogen; CN; CONR¹²R¹³, wherein R¹² and R¹³ are independently selected from H, C₁₋₃ alkoxy, branched C₃₋₆ alkyl, C₁₋₆ alkyl optionally substituted with a F, bicyclopentanyl, CH₂-cyclopropyl, and CH₂-cyclobutyl, or R¹² and R¹³ together with the nitrogen form a 5 or 6-membered ring containing one nitrogen and 4 or 5 carbon atoms; and C₁₋₃ alkyl substituted with a F. Typically, when B1 is pyridinyl and substituted with two groups, one is a halogen and the other is selected from halogen; CN; CONR¹²R¹³, wherein R¹² and R¹³ are independently selected from H, C₁₋₃ alkoxy, branched C₃₋₆ alkyl, C₁₋₆ alkyl optionally substituted with a F, bicyclopentanyl, CH₂-cyclopropyl, and CH₂-cyclobutyl, or R¹² and R¹³ together with the nitrogen form a 5 or 6-membered ring containing one nitrogen and 4 or 5 carbon atoms; C₁₋₃ alkyl substituted with a F.

In a still further embodiment B1 is selected from a phenyl, optionally substituted with a group selected from a halogen; CN; —CONR⁶R⁷, wherein R⁶ and R⁷ are independently selected from H, C₁₋₃ alkyl, cyclopropyl, and iso-propyl; and C₁₋₃ alkyl, optionally substituted with a F. Typically, B1 is selected from a phenyl, optionally substituted with a group selected from a Cl; F; Br; CN; CONHCH₃; and C₁₋₃ alkyl, optionally substituted with a F; such as phenyl substituted with one or two selected from Cl, Br, CN, and CONHCH₃, preferably phenyl is substituted with two selected from Cl, Br, CN, and CONHCH₃, such as phenyl is substituted with one halogen and one group selected from Cl, Br, CN, and CONHCH₃.

In a further embodiment B1 is selected from a C₁₋₆ alkyl or branched C₃₋₆ alkyl.

In a still further embodiment B1 is selected from a C₂_₆ alkynyl.

In a further embodiment R¹ is H.

In a still further embodiment R¹ is OH.

In a further embodiment R¹ is OC₁₋₄ alkyl, such as O-methyl, O-ethyl, or 0-isopropyl. Typically, R¹ is O-methyl.

In a still further embodiment R¹ is OC₁₋₄ alkyl substituted with at least one from the group consisting of phenyl and phenyl substituted with one or more groups selected form OH and halogen.

Preferably the D-galactopyranose compound of formula (1) is selected form any one of the compounds prepared in examples 1-43; or a pharmaceutically acceptable salt or solvate thereof.

The skilled person will understand that it may be necessary to adjust or change the order of steps in the processes a1 to a47, and such change of order is encompassed by the aspects of the process as described above in the reaction schemes and accompanying description of the process steps.

Furthermore, the skilled person will understand that the processes described above and hereinafter the functional groups of intermediate compounds may need to be protected by protecting groups.

Functional groups that it is desirable to protect include hydroxy, amino and carboxylic acid. Suitable protecting groups for hydroxy include optionally substituted and/or unsaturated alkyl groups (e.g. methyl, allyl, benzyl or tert-butyl), trialkyl silyl or diarylalkylsilyl groups (e.g. t-butyldimethylsilyl, t-butyldipheylsilyl or trimethylsilyl), AcO(acetoxy), TBS(t-butyldimethylsilyl), TMS(trimethylsilyl), PMB (p-methoxybensyl), and tetrahydropyranyl. Suitable protecting groups for carboxylic acid include (C₁-6)-alkyl or benzyl esters. Suitable protecting groups for amino include t-butyloxycarbonyl, benzyloxycarbonyl, 2-(trimethylsilyl)-ethoxy-methyl or 2-trimethylsilylethoxycarbonyl (Teoc). Suitable protecting groups for S include S-C(═N)NH₂, TIPS.

The protection and deprotection of functional groups may take place before or after any reaction in the above-mentioned processes.

Furthermore the skilled person will appreciate, that, in order to obtain compounds of the invention in an alternative, and on some occasions more convenient manner, the individual process steps mentioned hereinbefore may be performed in different order, and/or the individual reactions may be performed at a different stage in the overall route (i.e. substituents may be added to and/or chemical transformations performed upon, different intermediates to those mentioned hereinbefore in conjunction with a particular reaction). This may negate, or render necessary, the need for protecting groups.

In a still further embodiment the compound (1) is on free form. “On free form” as used herein means a compound of formula (1), either an acid form or base form, or as a neutral compound, depending on the substitutents. The free form does not have any acid salt or base salt in addition. In one embodiment the free form is an anhydrate. In another embodiment the free form is a solvate, such as a hydrate.

In a further embodiment the compound of formula (1) is a crystalline form. The skilled person may carry out tests in order to find polymorphs, and such polymorphs are intended to be encompassed by the term “crystalline form” as used herein.

When the compounds and pharmaceutical compositions herein disclosed are used for the above treatment, a therapeutically effective amount of at least one compound is administered to a mammal in need of said treatment.

The term “C₁₋ₓ alkyl” as used herein means an alkyl group containing 1-x carbon atoms, e.g. C₁₋₅ or C₁₋₆, such as methyl, ethyl, propyl, butyl, pentyl or hexyl.

The term “branched C₃₋₆ alkyl” as used herein means a branched alkyl group containing 3-6 carbon atoms, such as isopropyl, isobutyl, tert-butyl, isopentyl, 3-methylbutyl, 2,2-dimethylpropyl, n-hexyl, 2-methylpentyl, 2,2-dimethylbutyl, 2,3-dimethylbutyl.

The term “C₃-7 cycloalkyl” as used herein means a cyclic alkyl group containing 3-7 carbon atoms, such as cyclopropyl, cyclobutyl, cyclopentyl, cyclohexyl, cycloheptyl, and 1-methylcyclopropyl.

The term “C₅₋₇ cycloalkyl” as used herein means a cyclic alkyl group containing 5-7 carbon atoms, such as cyclopentyl, cyclohexyl, or cycloheptyl.

The term “C₂-alkynyl” as used herein means —CCH. Wherein the two carbons are connected by a triple bond.

The term “Oxo” as used herein means an oxygen atom with double bonds, also indicated as ═O.

The term “CN” as used herein means a nitril.

The term “a five or six membered heteroaromatic ring” as used herein means one five membered heteroaromatic ring or one six membered heteroaromatic ring. The five membered heteroaromatic ring contains 5 ring atoms of which one to four are heteroatoms selected from N, O, and S. The six membered heteroaromatic ring contains 6 ring atoms of which one to five are heteroatoms selected from N, O and S. Examples include thiophene, furan, pyran, pyrrole, imidazole, pyrazole, isothiazole, isooxazole, pyridine, pyrazine, pyrimidine and pyridazine. When such heteroaromatic rings are substituents they are termed thiophenyl, furanyl, pyranyl, pyrrolyl, imidazolyl, pyrazolyl, isothiazolyl, isooxazolyl, pyridinyl, pyrazinyl, pyrimidinyl and pyridazinyl. Also included are oxazoyl, thiazoyl, thiadiazoly, oxadiazoyl, and pyridonyl.

The term “a heterocycle, such as heteroaryl or heterocycloalkyl” as used herein means a heterocycle consisting of one or more 3-7 membered ring systems containing one or more heteroatoms and wherein such ring systems may optionally be aromatic. The term “a heteroaryl” as used herein means a mono or bicyclic aromatic ringsystem containing one or more heteroatoms, such as 1-10, e.g. 1-6, selected from O, S, and N, including but not limited to benzothiazolyl, oxazolyl, oxadiazolyl, thiophenyl, thiadiazolyl, thiazolyl, thiazolopyridinyl, pyridyl, pyrimidinyl, pyridonyl, pyrimidonyl, quinolinyl, azaquionolyl, isoquinolinyl, azaisoquinolyl, quinazolinyl, azaquinazolinyl, bensozazoyl, azabensoxazoyl, bensothiazoyl, or azabensothiazoyl.

The term “a heterocycloalkyl” as used herein means a mono or bicyclic 3-7 membered alifatic heterocycle containing one or more heteroatoms, such as 1-7, e.g. 1-5, selected from O, S, and N, including but not limited to azetidinyl, piperidinyl, tetrahydropyranyl, tetrahydrothipyranyl, or piperidonyl. The term “treatment” and “treating” as used herein means the management and care of a patient for the purpose of combating a condition, such as a disease or a disorder. The term is intended to include the full spectrum of treatments for a given condition from which the patient is suffering, such as administration of the active compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of a patient for the purpose of combating the disease, condition, or disorder and includes the administration of the active compounds to prevent the onset of the symptoms or complications. The treatment may either be performed in an acute or in a chronic way. The patient to be treated is preferably a mammal; in particular, a human being, but it may also include animals, such as dogs, cats, cows, sheep and pigs.

The term “a therapeutically effective amount” of a compound of formula (1) of the present invention as used herein means an amount sufficient to cure, alleviate or partially arrest the clinical manifestations of a given disease and its complications. An amount adequate to accomplish this is defined as “therapeutically effective amount”. Effective amounts for each purpose will depend on the severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician or veterinary.

In a still further aspect, the present invention relates to a pharmaceutical composition comprising the compound of formula (1) and optionally a pharmaceutically acceptable additive, such as a carrier or an excipient.

As used herein “pharmaceutically acceptable additive” is intended without limitation to include carriers, excipients, diluents, adjuvant, colorings, aroma, preservatives etc. that the skilled person would consider using when formulating a compound of the present invention in order to make a pharmaceutical composition.

The adjuvants, diluents, excipients and/or carriers that may be used in the composition of the invention must be pharmaceutically acceptable in the sense of being compatible with the compound of formula (1) and the other ingredients of the pharmaceutical composition, and not deleterious to the recipient thereof. It is preferred that the compositions shall not contain any material that may cause an adverse reaction, such as an allergic reaction. The adjuvants, diluents, excipients and carriers that may be used in the pharmaceutical composition of the invention are well known to a person skilled within the art.

As mentioned above, the compositions and particularly pharmaceutical compositions as herein disclosed may, in addition to the compounds herein disclosed, further comprise at least one pharmaceutically acceptable adjuvant, diluent, excipient and/or carrier. In some embodiments, the pharmaceutical compositions comprise from 1 to 99% by weight of said at least one pharmaceutically acceptable adjuvant, diluent, excipient and/or carrier and from 1 to 99% by weight of a compound as herein disclosed. The combined amount of the active ingredient and of the pharmaceutically acceptable adjuvant, diluent, excipient and/or carrier may not constitute more than 100% by weight of the composition, particularly the pharmaceutical composition.

In some embodiments, only one compound as herein disclosed is used for the purposes discussed above.

In some embodiments, two or more of the compounds as herein disclosed are used in combination for the purposes discussed above.

The composition, particularly pharmaceutical composition comprising a compound set forth herein may be adapted for oral, intravenous, topical, intraperitoneal, nasal, buccal, sublingual, or subcutaneous administration, or for administration via the respiratory tract in the form of, for example, an aerosol or an air-suspended fine powder. Therefore, the pharmaceutical composition may be in the form of, for example, tablets, capsules, powders, nanoparticles, crystals, amorphous substances, solutions, transdermal patches or suppositories.

Further embodiments of the process are described in the experimental section herein, and each individual process as well as each starting material constitutes embodiments that may form part of embodiments.

The above embodiments should be seen as referring to any one of the aspects (such as ‘method for treatment’, ‘pharmaceutical composition’, ‘compound for use as a medicament’, or ‘compound for use in a method’) described herein as well as any one of the embodiments described herein unless it is specified that an embodiment relates to a certain aspect or aspects of the present invention.

All references, including publications, patent applications and patents, cited herein are hereby incorporated by reference to the same extent as if each reference was individually and specifically indicated to be incorporated by reference and was set forth in its entirety herein.

All headings and sub-headings are used herein for convenience only and should not be construed as limiting the invention in any way.

Any combination of the above-described elements in all possible variations thereof is encompassed by the invention unless otherwise indicated herein or otherwise clearly contradicted by context.

The terms “a” and “an” and “the” and similar referents as used in the context of describing the invention are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (e.g., all exact exemplary values provided with respect to a particular factor or measurement can be considered to also pro-vide a corresponding approximate measurement, modified by “about,” where appropriate).

All methods described herein can be performed in any suitable order unless otherwise indicated herein or otherwise clearly contradicted by context.

The use of any and all examples, or exemplary language (e.g., “such as”) provided herein, is intended merely to better illuminate the invention and does not pose a limitation on the scope of the invention unless otherwise indicated. No language in the specification should be construed as indicating any element is essential to the practice of the invention unless as much is explicitly stated.

The citation and incorporation of patent documents herein is done for convenience only and does not reflect any view of the validity, patentability and/or enforceability of such patent documents.

The term “and/or” as used herein is intended to mean both alternatives as well as each of the alternatives individually. For instance, the expression “xxx and/or yyy” means “xxx and yyy”; “xxx”; or “yyy”, all three alternatives are subject to individual embodiments.

The description herein of any aspect or embodiment of the invention using terms such as “comprising”, “having”, “including” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

The present invention is further illustrated by the following examples that, however, are not to be construed as limiting the scope of protection. The features disclosed in the foregoing description and in the following examples may, both separately and in any combination thereof, be material for realizing the invention indiverse forms thereof.

### Experimental Procedures (Evaluation of Kd Values)

The affinity of Example 1-43 for galectins were determined by a fluorescence anisotropy assay where the compound was used as an inhibitor of the interaction between galectin and a fluorescein tagged saccharide probe as described Sörme, P., Kahl-Knutsson, B., Huflejt, M., Nilsson, U. J., and Leffler H. (2004) Fluorescence polarization as an analytical tool to evaluate galectin-ligand interactions. Anal. Biochem. 334: 36-47, (Sörme et al., 2004) and Monovalent interactions of Galectin-1 By Salomonsson, Emma; Larumbe, Amaia; Tejler, Johan; Tullberg, Erik; Rydberg, Hanna; Sundin, Anders; Khabut, Areej; Frejd, Torbjomn; Lobsanov, Yuri D.; Rini, James M.; et al, From Biochemistry (2010), 49(44), 9518-9532, (Salomonsson et al., 2010).

| | | | Galectin-1 | Galectin-3 |
|---|---|---|---|---|
| Example | Name | Structure | Kd (μM) | Kd (μM) |
| 1 | 5-Bromo-2-(N-methyl-carbonyl)phenyl 3-[4-(3- chloro-1H-1,2-pyrazol-1-yl)-1H-1,2,3-triazol-1- yl]-3-deoxy-2-O-methyl-1-thio-α-D- galactopyranoside | 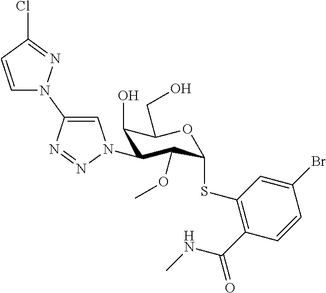 | 0.057 | 0.16 |
| 2 | 5-Bromo-2-cyanophenyl 3-[4-(3-chloro-1H-1,2- pyrazol-1-yl)-1H-1,2,3-triazol-1-yl]-3-deoxy-2- O-methyl-1-thio-α-D-galactopyranoside | 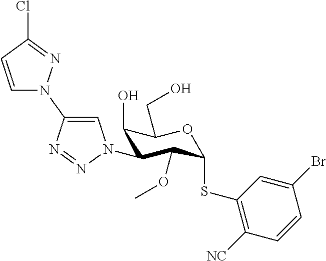 | 0.085 | 0.13 |
| 3 | 5-Bromo-2-cyanopyridin-3-yl 3-[4-(3-chloro-1H- 1,2-pyrazol-1-yl)-1H-1,2,3-triazol-1-yl]-3-deoxy- 2-O-methyl-1-thio-α-D-galactopyranoside | 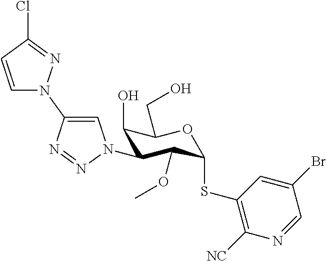 | 0.13 | 0.12 |
| 4 | 5-Chloropyridin-3-yl 3-deoxy-3-[4-(3-fluoro-1H- 1,2-pyrazol-1-yl)-1H-1,2,3-triazol-1-yl]-1-thio-α- D-galactopyranoside | 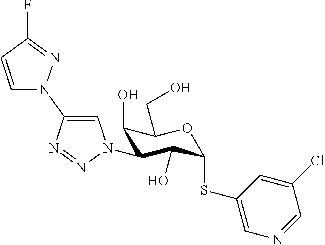 | 1.7 | 0.36 |
| 5 | 5-Chloropyridin-3-yl 3-[4-(3-chloro-1H-1,2- pyrazol-1-yl)-1H-1,2,3-triazol-1-yl]-3-deoxy-1- thio-α-D-galactopyranoside | 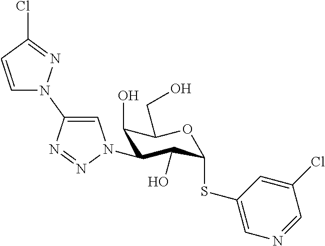 | 0.50 | 0.31 |
| 6 | 5-Chloro-2-(trifluoromethyl)pyridin-3-yl 3-[4-(3- chloro-1H-1,2-pyrazol-1-yl)-1H-1,2,3-triazol-1- yl]-3-deoxy-2-O-methyl-1-thio-α-D- galactopyranoside | 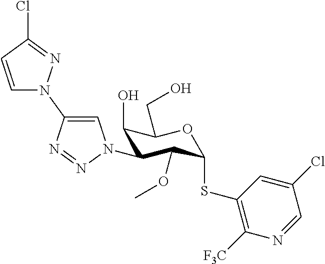 | 0.20 | 0.27 |
| 7 | 5-Bromo-2-(trifluoromethyl)pyridin-3-yl 3-[4-(3- chloro-1H-1,2-pyrazol-1-yl)-1H-1,2,3-triazol-1- yl]-3-deoxy-2-O-methyl-1-thio-α-D- galactopyranoside | 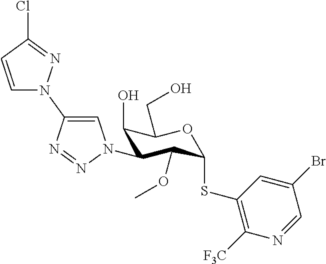 | 0.15 | 0.21 |
| 8 | 3-Chloro-2-(trifluoromethyl)pyridin-5-yl 3-[4-(3- chloro-1H-1,2-pyrazol-1-yl)-1H-1,2,3-triazol-1- yl]-3-deoxy-2-O-methyl-1-thio-α-D- galactopyranoside | 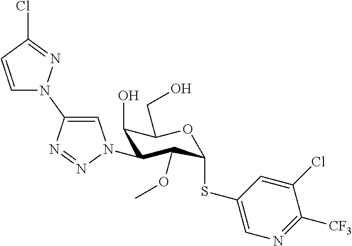 | 0.36 | 0.54 |
| 9 | 3-Bromo-2-(trifluoromethyl)pyridin-5-yl 3-[4-(3- chloro-1H-1,2-pyrazol-1-yl)-1H-1,2,3-triazol-1- yl]-3-deoxy-2-O-methyl-1-thio-α-D- galactopyranoside | 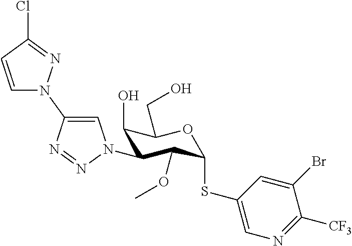 | 0.21 | 0.39 |
| 10 | 5-Bromo-2-(N,N-dimethylcarbamoyl)pyridin-3- yl 3-[4-(3-chloro-1H-1,2-pyrazol-1-yl)-1H-1,2,3- triazol-1-yl]-3-deoxy-2-O-methyl-1-thio-α-D- galactopyranoside | 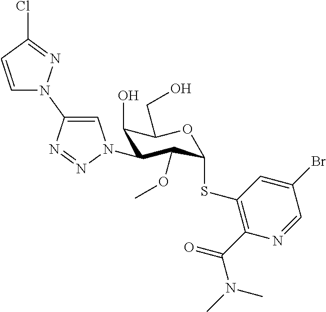 | 0.054 | 0.073 |
| 11 | 5-Bromo-2-cyanopyridin-3-yl 3-[4-(4-chloro-1H- 1,2-pyrazol-1-yl)-1H-1,2,3-triazol-1-yl]-3-deoxy- 2-O-methyl-1-thio-α-D-galactopyranoside | 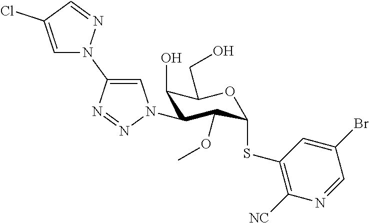 | 0.52 | 0.42 |
| 12 | 5-Bromo-2-cyanopyridin-3-yl 3-deoxy-3-[4-(3- fluoro-1H-1,2-pyrazol-1-yl)-1H-1,2,3-triazol-1- yl]-2-O-methyl-1-thio-α-D-galactopyranoside | 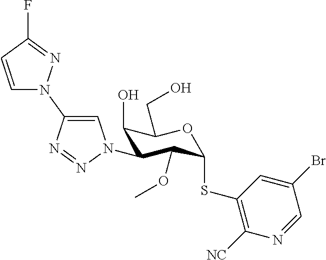 | 0.18 | 0.16 |
| 13 | 5-Bromo-2-cyanopyridin-3-yl 3-deoxy-3-[4-(4- fluoro-1H-1,2-pyrazol-1-yl)-1H-1,2,3-triazol-1- yl]-2-O-methyl-1-thio-α-D-galactopyranoside | 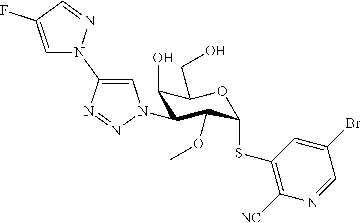 | 0.18 | 1.7 |
| 14 | 5-Bromo-2-cyanopyridin-3-yl 3-deoxy-3-[4-(3- methyl-1H-1,2-pyrazol-1-yl)-1H-1,2,3-triazol-1- yl]-2-O-methyl-1-thio-α-D-galactopyranoside | 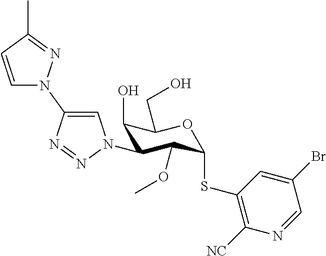 | 0.18 | 1.4 |
| 15 | 5-Bromo-2-cyanopyridin-3-yl 3-deoxy-3-[4-(5- methyl-1H-1,2-pyrazol-1-yl)-1H-1,2,3-triazol-1- yl]-2-O-methyl-1-thio-α-D-galactopyranoside | 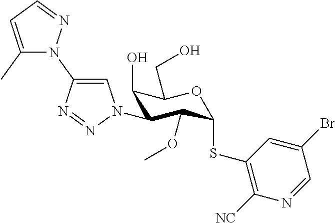 | 0.89 | 4.3 |
| 16 | 5-Bromo-2-cyanopyridin-3-yl 3-[4-(3-chloro-5- methyl-1H-1,2-pyrazol-1-yl)-1H-1,2,3-triazol-1- yl]-3-deoxy-2-O-methyl-1-thio-α-D- galactopyranoside | 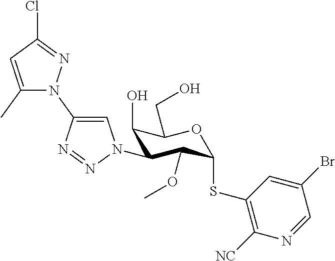 | 0.35 | 0.40 |
| 17 | 5-Bromo-2-cyanopyridin-3-yl 3-{4-[5-chloro-3- (trifluoromethyl)-1H-1,2-pyrazol-1-yl]-1H-1,2,3- triazol-1-yl}-3-deoxy-2-O-methyl-1-thio-α-D- galactopyranoside | 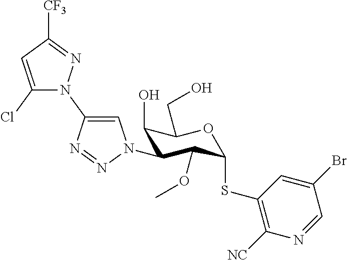 | 2.0 | 0.56 |
| 18 | 5-Bromo-2-cyanopyridin-3-yl 3-[4-(3-chloro-4- methyl-1H-1,2-pyrazol-1-yl)-1H-1,2,3-triazol-1- yl]-3-deoxy-2-O-methyl-1-thio-α-D- galactopyranoside | 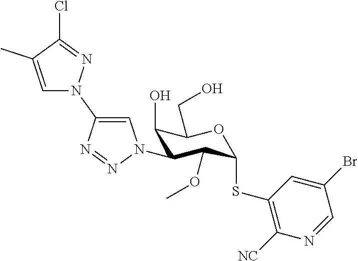 | 0.35 | 0.041 |
| 19 | 5-Bromo-2-(N,N- ethylisopropylcarbamoyl)pyridin-3-yl 3-[4-(3- chloro-1H-1,2-pyrazol-1-yl)-1H-1,2,3-triazol-1- yl]-3-deoxy-2-O-methyl-1-thio-α-D- galactopyranoside | 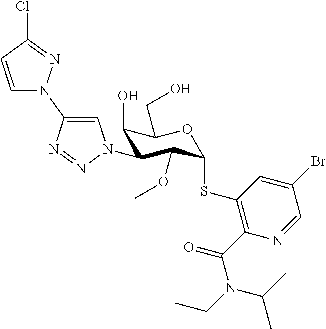 | 0.11 | 0.092 |
| 20 | 5-Bromo-2-(N,N-diisobutylcarbamoyl)pyridin-3- yl 3-[4-(3-chloro-1H-1,2-pyrazol-1-yl)-1H-1,2,3- triazol-1-yl]-3-deoxy-2-O-methyl-1-thio-α-D- galactopyranoside | 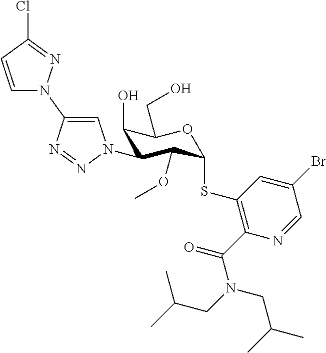 | 0.36 | 0.36 |
| 21 | 5-Bromo-2-[N,N- (cyclopropylmethyl)ethylcarbamoyl]pyridin-3-yl 3-[4-(3-chloro-1H-1,2-pyrazol-1-yl)-1H-1,2,3- triazol-1-yl]-3-deoxy-2-O-methyl-1-thio-α-D- galactopyranoside | 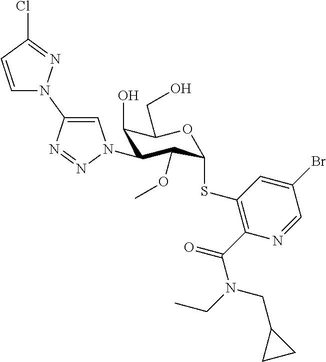 | 0.10 | 0.10 |
| 22 | 5-Bromo-2-[N,N-(2-fluoro-2- methylpropyl)methylcarbamoyl]pyridin-3-yl 3- [4-(3-chloro-1H-1,2-pyrazol-1-yl)-1H-1,2,3- triazol-1-yl]-3-deoxy-2-O-methyl-1-thio-α-D- galactopyranoside | 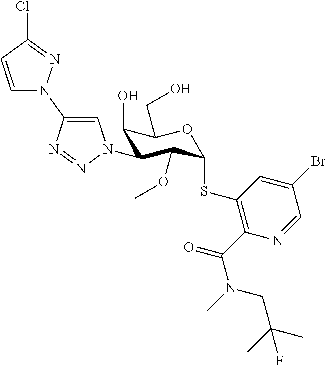 | 0.098 | 0.076 |
| 23 | 5-Bromo-2-[N,N-(tert- butyl)ethylcarbamoyl]pyridin-3-yl 3-[4-(3- chloro-1H-1,2-pyrazol-1-yl)-1H-1,2,3-triazol-1- yl]-3-deoxy-2-O-methyl-1-thio-α-D- galactopyranoside | 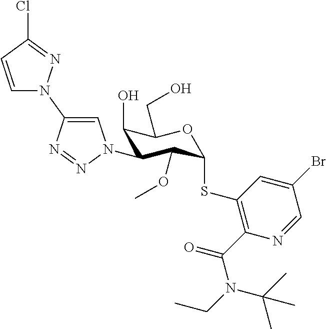 | 0.087 | 0.072 |
| 24 | 5-Bromo-2-[N,N- bis(cyclopropylmethyl)carbamoyl]pyridin-3-yl 3- [4-(3-chloro-1H-1,2-pyrazol-1-yl)-1H-1,2,3- triazol-1-yl]-3-deoxy-2-O-methyl-1-thio-α-D- galactopyranoside | 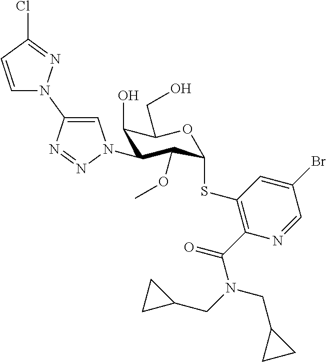 | 0.12 | 0.083 |
| 25 | 5-Bromo-2-[N,N-(cyclobutylmethyl)ethyl carbamoyl}pyridin-3-yl 3-[4-(3-chloro-1H-1,2- pyrazol-1-yl)-1H-1,2,3-triazol-1-yl]-3-deoxy-2- O-methyl-1-thio-α-D-galactopyranoside | 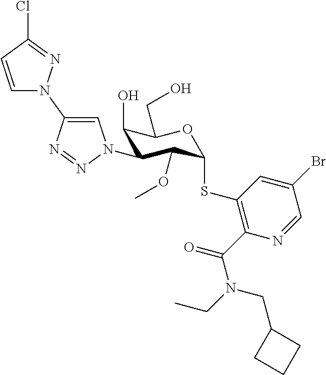 | 0.14 | 0.075 |
| 26 | 5-Bromo-2-[N,N- (cyclobutylmethyl)isopropylcarbamoyl]pyridin-3- yl 3-[4-(3-chloro-1H-1,2-pyrazol-1-yl)-1H-1,2,3- triazol-1-yl]-3-deoxy-2-O-methyl-1-thio-α-D- galactopyranoside | 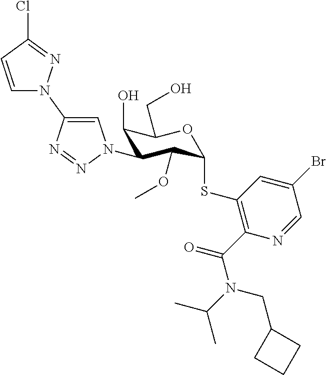 | 0.16 | 0.11 |
| 27 | 5-Bromo-2-[N,N- bis(cyclobutylmethyl)carbamoyl]pyridin-3-yl 3- [4-(3-chloro-1H-1,2-pyrazol-1-yl)-1H-1,2,3- triazol-1-yl]-3-deoxy-2-O-methyl-1-thio-α-D- galactopyranoside | 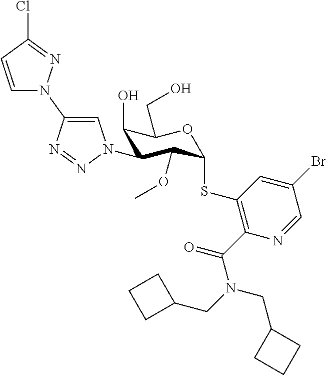 | 0.38 | 0.26 |
| 28 | 5-Bromo-2-(pyrrolidine-1-carbonyl)pyridin-3-yl 3-[4-(3-chloro-1H-1,2-pyrazol-1-yl)-1H-1,2,3- triazol-1-yl]-3-deoxy-2-O-methyl-1-thio-α-D- galactopyranoside | 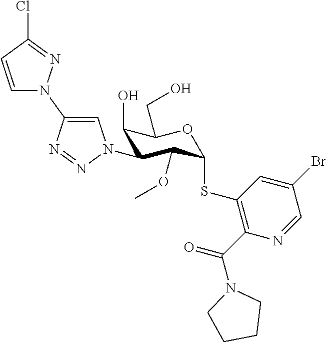 | 0.069 | 0.052 |
| 29 | 5-Bromo-2-[N,N-ethyl-(2,2,2- trifluoroethyl)carbamoyl]pyridin-3-yl 3-[4-(3- chloro-1H-1,2-pyrazol-1-yl)-1H-1,2,3-triazol-1- yl]-3-deoxy-2-O-methyl-1-thio-α-D- galactopyranoside | 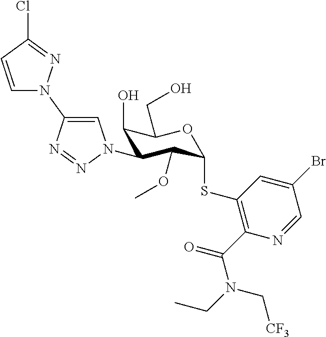 | 0.10 | 0.060 |
| 30 | 5-Bromo-2-[N,N-ethyl(2-fluoro-2- methylpropyl)carbamoyl]pyridin-3-yl 3-[4-(3- chloro-1H-1,2-pyrazol-1-yl)-1H-1,2,3-triazol-1- yl]-3-deoxy-2-O-methyl-1-thio-α-D- galactopyranoside | 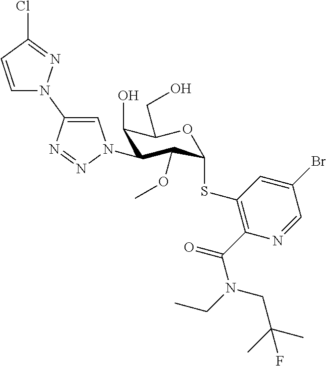 | 0.14 | 0.062 |
| 31 | 5-Bromo-2-[N,N- (cyclopropylmethyl)isopropylcarbamoyl]pyridin- 3-yl 3-[4-(3-chloro-1H-1,2-pyrazol-1-yl)-1H- 1,2,3-triazol-1-yl]-3-deoxy-2-O-methyl-1-thio-α- D-galactopyranoside | 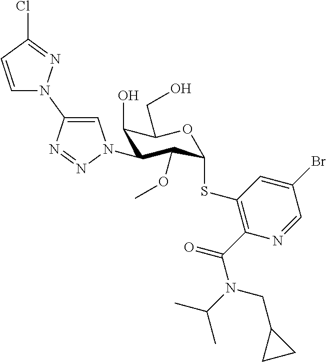 | 0.072 | 0.060 |
| 32 | 5-Bromo-2-(N,N- isobutylisopropylcarbamoyl)pyridin-3-yl 3-[4-(3- chloro-1H-1,2-pyrazol-1-yl)-1H-1,2,3-triazol-1- yl]-3-deoxy-2-O-methyl-1-thio-α-D- galactopyranoside | 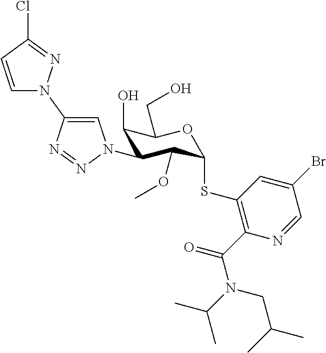 | 0.11 | 0.063 |
| 33 | 5-Bromo-2-[N,N- (cyclopropylmethyl)methoxycarbamoyl]pyridin- 3-yl 3-[4-(3-chloro-1H-1,2-pyrazol-1-yl)-1H- 1,2,3-triazol-1-yl]-3-deoxy-2-O-methyl-1-thio-α- D-galactopyranoside | 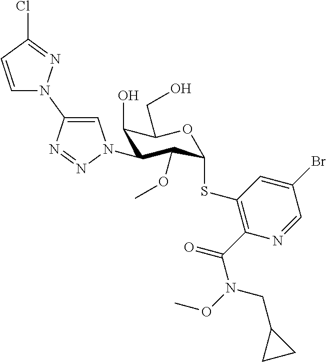 | 0.074 | 0.068 |
| 34 | 5-Bromo-2-(N-methylcarbamoyl)pyridin-3-yl 3- [4-(3-chloro-1H-1,2-pyrazol-1-yl)-1H-1,2,3- triazol-1-yl]-3-deoxy-2-O-methyl-1-thio-α-D- galactopyranoside | 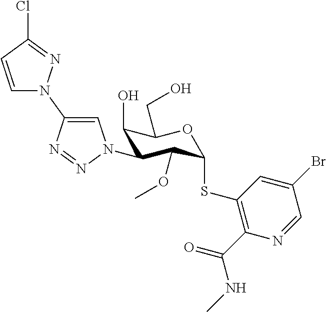 | 0.063 | 0.14 |
| 35 | 5-Bromo-2-(N-ethylcarbamoyl)pyridin-3-yl 3-[4- (3-chloro-1H-1,2-pyrazol-1-yl)-1H-1,2,3-triazol- 1-yl]-3-deoxy-2-O-methyl-1-thio-α-D- galactopyranoside | 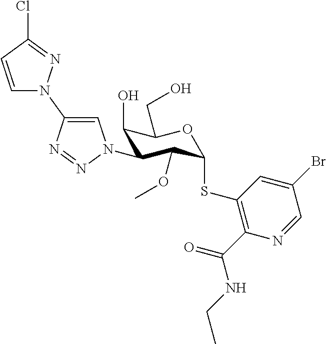 | 0.063 | 0.14 |
| 36 | 5-Bromo-2-(N-butylcarbamoyl)pyridin-3-yl 3-[4- (3-chloro-1H-1,2-pyrazol-1-yl)-1H-1,2,3-triazol- 1-yl]-3-deoxy-2-O-methyl-1-thio-α-D- galactopyranoside | 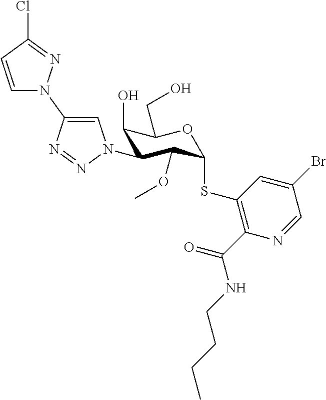 | 0.11 | 0.14 |
| 37 | 5-Bromo-2-(N-isobutylcarbamoyl)pyridin-3-yl 3- [4-(3-chloro-1H-1,2-pyrazol-1-yl)-1H-1,2,3- triazol-1-yl]-3-deoxy-2-O-methyl-1-thio-α-D- galactopyranoside | 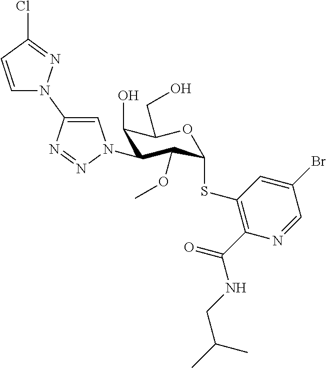 | 0.092 | 0.11 |
| 38 | 5-Bromo-2-[N-(2-fluoroethyl)carbamoyl]pyridin- 3-yl 3-[4-(3-chloro-1H-1,2-pyrazol-1-yl)-1H- 1,2,3-triazol-1-yl]-3-deoxy-2-O-methyl-1-thio-α- D-galactopyranoside | 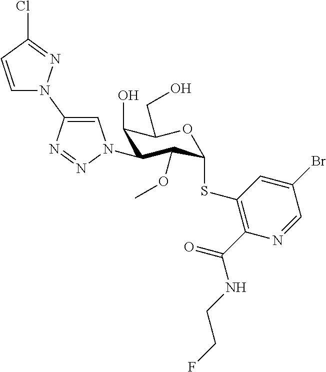 | 0.080 | 0.18 |
| 39 | 5-Bromo-2-(N-bicyclo[1.1.1]pentan-1- ylcarbamoyl)pyridin-3-yl 3-[4-(3-chloro-1H-1,2- pyrazol-1-yl)-1H-1,2,3-triazol-1-yl]-3-deoxy-2- O-methyl-1-thio-α-D-galactopyranoside | 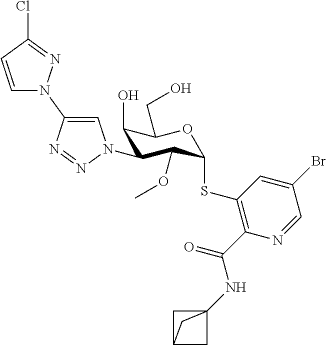 | 0.064 | 0.18 |
| 40 | 5-Bromo-2-(N-cyclobutylcarbamoyl)pyridin-3-yl 3-[4-(3-chloro-1H-1,2-pyrazol-1-yl)-1H-1,2,3- triazol-1-yl]-3-deoxy-2-O-methyl-1-thio-α-D- galactopyranoside | 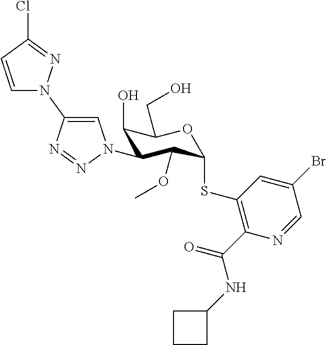 | 0.082 | 0.16 |
| 41 | 5-Bromo-2-(N-cyclopropylcarbamoyl)pyridin-3- yl 3-[4-(3-chloro-1H-1,2-pyrazol-1-yl)-1H-1,2,3- triazol-1-yl]-3-deoxy-2-O-methyl-1-thio-α-D- galactopyranoside | 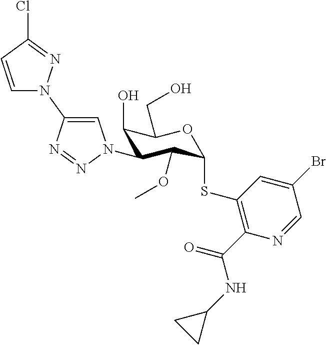 | 0.073 | 0.16 |
| 42 | 5-Bromo-2-cyanopyridin-3-yl 3-[4-(3,4-dichloro- 1H-1,2-pyrazol-1-yl)-1H-1,2,3-triazol-1-yl]-3- deoxy-2-O-methyl-1-thio-α-D-galactopyranoside | 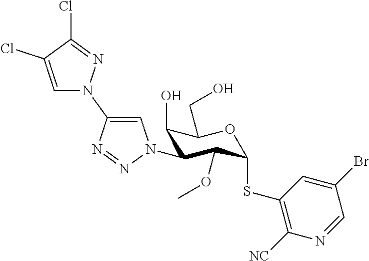 | 0.29 | 0.030 |
| 43 | 5-Bromo-2-cyanopyridin-3-yl 3-[4-(3-chloro-4- fluoro-1H-1,2-pyrazol-1-yl)-1H-1,2,3-triazol-1- yl]-3-deoxy-2-O-methyl-1-thio-α-D- galactopyranoside | 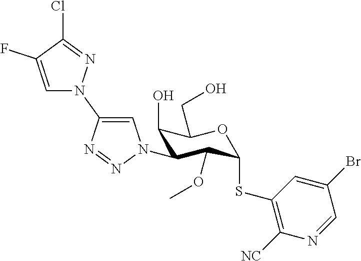 | 0.27 | 0.14 |

### SYNTHESIS OF EXAMPLES AND INTERMEDIATES

### General Experimental

Nuclear Magnetic Resonance (NMR) spectra were recorded on a 400 MHz Bruker AVANCE III 500 instrument or a Varian instrument at 400 MHz, at 25° C. Chemical shifts are reported in ppm (d) using the residual solvent as internal standard. Peak multiplicities are expressed as follow: s, singlet; d, doublet; dd, doublet of doublets; t, triplet; dt, doublet of triplet; q, quartet; m, multiplet; br s, broad singlet. In the case of anomeric mixtures, the shifts of the individual anomers are reported separately and the α/β ratio was calculated based on the integration of the anomeric peaks.

LC-MS were acquired on an Agilent 1200 HPLC coupled with an Agilent MSD mass spectrometer operating in ES (+) ionization mode. Column: XBridge C18 (4.6×50 mm, 3.5 μm) or SunFire C18 (4.6×50 mm, 3.5 μm). Solvent A water+0.1% TFA and solvent B Acetonitrile+0.1% TFA or solvent A water (10 mM Ammonium hydrogen carbonate) and solvent B Acetonitrile. Wavelength: 254 nM. Alternatively, LC-MS were acquired on an Agilent 1100 HPLC coupled with an Agilent MSD mass spectrometer operating in ES (+) ionization mode. Column: Waters symmetry 2.1×30 mm C18 or Chromolith RP-18 2×50 mm. Solvent A water+0.1% TFA and solvent B Acetonitrile+0.1% TFA. Wavelength 254 nm.

Preparative HPLC was performed on a Gilson 281. Flow: 20 mL/min Column: X-Select 10 μm 19×250 mm column or Gemini 5 μm NX-C18 110 Å 21.2×150 mm. Wavelength: 254 nm, 220 nm or 214 nm. 1) Solvent A water (0.1% TFA) and solvent B Acetonitrile or 2) Solvent A water (10 mM Ammonium hydrogen carbonate) and solvent B Acetonitrile or 3) Solvent A water (0.1% Formic acid) and solvent B Acetonitrile or 4) Solvent A water (0.2% Ammonium hydroxide) and solvent B Acetonitrile. Alternatively, preparative HPLC was performed on a Gilson 215. Flow: 25 m/min Column: XBrige prep C18 10 μm OBD (19×250 mm) column. Wavelength: 254 nM. Solvent A water (10 mM Ammonium hydrogen carbonate) and solvent B Acetonitrile. Alternatively, preparative HPLC were acquired on a Gilson system. Flow: 15 ml/min Column: kromasil 100-5-C18 column. Wavelength: 220 nm. Solvent A water+0.1% TFA and solvent B Acetonitrile+0.1% TFA.

The following abbreviations are used
aq: aqueous
Calcd: Calculated
MeCN: Acetonitrile
CuI: Copper Iodide
DCM: Dichloromethane
DIPEA: Diisopropylethylamine
DMF: N,N-dimethylformamide
ESI-MS: Electrospray ionization mass spectrometry
EtOAc or EA: Ethylacetate
Et₃N: Triethylamine
GC: Gas chromatography
h: hour(s)
HATU: 1-[Bis(dimethylamino)methylene]-1H-1,2,3-triazolo[4,5-b]pyridinium 3-oxid hexafluorophosphate
HPLC: High performance liquid chromatography
LC: Liquid Chromatography
MeCN: Acetonitrile
mL: milliliter
MeOH: Methanol
MeOD: Deuterated methanol
mm: millimeter
mM: millimolar
MS: Mass spectroscopy
nm: nanometer
NaOMe: Sodium methoxide
N₂: Nitrogen gas
NMR: Nuclear magnetic resonance
PE: petroleum ether
pH: acidity
Prep: Preparative
rt: Room temperature
TBAF: Tetrabutylammonium fluoride
TFA: trifluoroacetic acid
THF: Tetrahydrofuran
TIPS: Triisopropylsilyl
TMS: Trimethylsilyl
UV: Ultraviolet
Å: Ångström

### Synthesis of Example 1-43 from their Respective Intermediates 1-43

It should be noted that groups such as amides may due to the substitution pattern have a high barrier of rotation yielding rotameres that can be observed on for example the NMR time scale. For any such example the NMR spectra is reported as observed

### Example 1

### 5-Bromo-2-(N-methyl-carbonyl)phenyl 3-[4-(3-chloro-1H-1,2-pyrazol-1-yl)-1H-1,2,3-triazol-1-yl]-3-deoxy-2-O-methyl-1-thio-α-D-galactopyranoside

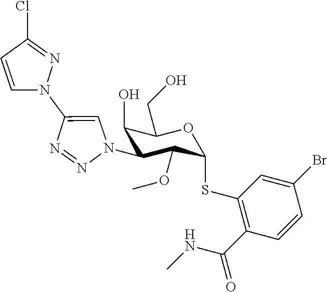

To a solution of 5-bromo-2-(N-methyl-carbonyl)phenyl 3-azido-3-deoxy-2-O-methyl-1-thio-α-D-galactopyranoside (30 mg, 0.067 mmol), CuI (2.6 mg, 0.013 mmol) and 2-(3-chloropyrazol-1-yl)ethynyl(triisopropyl)silane (17 mg, 0.084 mmol) in MeCN (1 mL) DIPEA (34 μL, 0.20 mmol) was added followed by TBAF (17 μL, 1 M in THF, 0.017 mmol) and the mixture was stirred 4 h at 50° C. The mixture was purified by prep HPLC (C₁₈, H₂O/MeCN/0.1% TFA) to give the product as a tetrabutylammonium salt. The product was filtered through a SCX column using MeOH to remove the tetrabutylammonium and afford the title compound (5 mg, 13%). ESI-MS m/z calcd for [C₂₀H₂₂BrClN₆O₅S] [M+H]⁺: 573.0; found: 573.0. ¹H NMR (500 MHz, Methanol-d₄) δ 8.34 (s, 1H), 8.24 (d, J=2.6 Hz, 1H), 8.00 (d, J=1.8 Hz, 1H), 7.55 (dd, J=8.2, 1.9 Hz, 1H), 7.32 (d, J=8.2 Hz, 1H), 6.51 (d, J=2.6 Hz, 1H), 6.17 (d, J=5.4 Hz, 1H), 5.00 (dd, J=11.4, 2.9 Hz, 1H), 4.58 (dd, J=11.4, 5.4 Hz, 1H), 4.48 (t, J=6.2 Hz, 1H), 4.20 (d, J=2.5 Hz, 1H), 3.76-3.65 (m, 2H), 3.39 (s, 3H), 2.91 (s, 3H).

### Example 2

### 5-Bromo-2-cyanophenyl 3-[4-(3-chloro-1H-1,2-pyrazol-1-yl)-1H-1,2,3-triazol-1-yl]-3-deoxy-2-O-methyl-1-thio-α-D-galactopyranoside

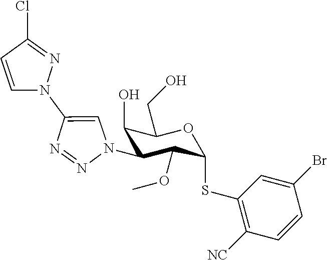

To a solution of 5-bromo-2-cyanophenyl 4,6-di-O-acetyl-3-azido-3-deoxy-2-O-methyl-1-thio-α-D-galactopyranoside (29 mg, 0.058 mmol), CuI (2.2 mg, 0.012 mmol) and 2-(3-chloropyrazol-1-yl)ethynyl(triisopropyl)silane (14 mg, 0.073 mmol) in MeCN (1 mL) DIPEA (30 μL, 0.17 mmol) was added followed by TBAF (15 μL, 1 M in THF, 0.015 mmol) and the mixture was stirred 3 h at 50° C. The mixture was partitioned between EtOAc and water. The organic phase was dried, evaporated and purified by chromatography (SiO₂, PE/EtOAc). The obtained material was stirred 2 h at rt in MeOH (1 mL) and NaOMe (0.1 mL, 1 M). The mixture was concentrated and purified by prep HPLC (C₁₈, H₂O/MeCN/0.1% TFA) to afford the title compound (3 mg, 10%). ESI-MS m/z calcd for [C₁₉H₁₈BrClN₆O₄S] [M+H]⁺: 541.0; found: 541.0. ¹H NMR (400 MHz, Methanol-d₄) δ 8.37 (s, 1H), 8.25 (d, J=2.6 Hz, 1H), 8.15 (s, 1H), 7.68 (d, J=1.6 Hz, 2H), 6.51 (d, J=2.6 Hz, 1H), 6.39 (d, J=5.3 Hz, 1H), 5.06 (dd, J=11.3, 2.9 Hz, 1H), 4.67 (dd, J=11.3, 5.3 Hz, 1H), 4.44 (t, J=6.1 Hz, 1H), 4.23 (d, J=2.5 Hz, 1H), 3.71 (dd, J=11.5, 5.6 Hz, 1H), 3.65 (dd, J=11.5, 6.6 Hz, 1H), 3.46 (s, 3H).

### Example 3

### 5-Bromo-2-cyanopyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside

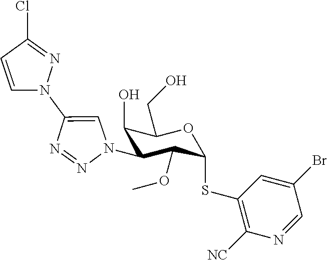

To a solution of 5-bromo-2-cyanopyridin-3-yl 4,6-di-O-acetyl-3-azido-3-deoxy-2-O-methyl-1-thio-α-D-galactopyranoside (92 mg, 0.18 mmol), CuI (7.0 mg, 0.037 mmol) and 2-(3-chloropyrazol-1-yl)ethynyl(triisopropyl)silane (52 mg, 0.18 mmol) in MeCN (2.0 mL) DIPEA (94 μL, 0.55 mmol) was added followed by TBAF (55 μL, 1 M in THF, 0.055 mmol) and the mixture was stirred 5 h at 50° C. The mixture was partitioned between EtOAc and brine. The organic phase was dried, evaporated and purified by chromatography (SiO₂, PE/EtOAc). The obtained material was stirred 6 h at rt in MeOH/Et₃N/water (9/3/1, 2.0 mL). The mixture was concentrated and purified by prep HPLC (C₁₈, H₂O/MeCN/0.1% TFA) to afford the title compound (16 mg, 16%). ESI-MS m/z calcd for [C₁₈H₁₇BrClN₇O₄S] [M+H]⁺: 542.0; found: 542.0. ¹H NMR (400 MHz, Methanol-d₄) δ 8.71 (d, J=2.0 Hz, 1H), 8.62 (d, J=2.0 Hz, 1H), 8.38 (s, 1H), 8.25 (d, J=2.6 Hz, 1H), 6.51 (d, J=2.6 Hz, 1H), 6.50 (d, J=5.3 Hz, 1H), 5.07 (dd, J=11.3, 2.9 Hz, 1H), 4.70 (dd, J=11.3, 5.3 Hz, 1H), 4.40 (t, J=6.0 Hz, 1H), 4.22 (d, J=2.5 Hz, 1H), 3.69 (d, J=6.0 Hz, 2H), 3.47 (s, 3H).

### Example 4

### 5-Chloropyridin-3-yl 3-deoxy-3-[4-(3-fluoro-1H-1,2-pyrazol-1-yl)-1H-1,2,3-triazol-1-yl]-1-thio-α-D-galactopyranoside

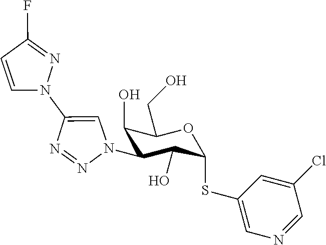

To a solution of 5-chloropyridin-3-yl 2,4,6-tri-O-acetyl-3-azido-3-deoxy-1-thio-α-D-galactopyranoside (50 mg, 0.11 mmol), CuI (4.2 mg, 0.022 mmol) and 2-(3-fluoropyrazol-1-yl)ethynyl(triisopropyl)silane (36 mg, 0.14 mmol) in MeCN (1.5 mL) DIPEA (56 μL, 0.33 mmol) was added followed by TBAF (27 μL, 1 M in THF, 0.027 mmol) and the mixture was stirred 4 h at 50° C. The mixture was partitioned between EtOAc and water and the aqueous phase was extracted with EtOAc. The combined organic phases were dried, evaporated and purified by chromatography (SiO₂, PE/EtOAc). The obtained material was stirred 1 h at rt in MeOH (2 mL) and NaOMe (0.3 mL, 1 M). The mixture was quenched with acetic acid (0.1 mL), concentrated, and purified by prep HPLC (C₁₈, H₂O/MeCN/0.1% TFA) to afford the title compound (10 mg, 21%). ESI-MS m/z calcd for [C₁₆H₁₆ClFN₆O₄S] [M+H]⁺: 443.1; found: 443.0. ¹H NMR (500 MHz, Methanol-d₄) δ 8.66 (d, J=1.6 Hz, 1H), 8.49 (d, J=2.0 Hz, 1H), 8.24 (s, 1H), 8.22 (t, J=2.1 Hz, 1H), 8.14 (t, J=2.6 Hz, 1H), 6.17 (dd, J=5.6, 2.7 Hz, 1H), 5.93 (d, J=5.3 Hz, 1H), 5.00 (dd, J=11.4, 2.8 Hz, 1H), 4.91 (dd, J=11.4, 5.3 Hz, 1H), 4.47 (t, J=6.3 Hz, 1H), 4.22-4.20 (m, 1H), 3.75-3.66 (m, 2H).

### Example 5

### 5-Chloropyridin-3-yl 3-[4-(3-chloro-1H-1,2-pyrazol-1-yl)-1H-1,2,3-triazol-1-yl]-3-deoxy-1-thio-α-D-galactopyranoside

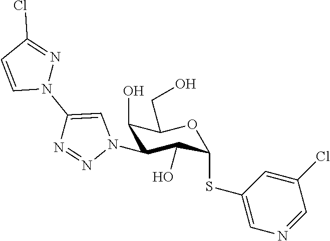

To a solution of 5-chloropyridin-3-yl 2,4,6-tri-O-acetyl-3-azido-3-deoxy-1-thio-α-D-galactopyranoside (41 mg, 0.088 mmol), CuI (3.4 mg, 0.018 mmol) and 2-(3-chloropyrazol-1-yl)ethynyl(triisopropyl)silane (25 mg, 0.088 mmol) in MeCN (1.5 mL) DIPEA (45 μL, 0.27 mmol) was added followed by TBAF (22 μL, 1 M in THF, 0.022 mmol) and the mixture was stirred, first 5 h at 50° C. then 3 days at rt. The mixture was partitioned between EtOAc and water and the aqueous phase was extracted with EtOAc. The combined organic phases were dried, evaporated and purified by chromatography (SiO₂, PE/EtOAc). The obtained material was stirred 1 h at rt in MeOH (2 mL) and NaOMe (0.3 mL, 1 M). The mixture was quenched with acetic acid (0.1 mL), concentrated, and purified by prep HPLC (C₁₈, H₂O/MeCN/0.1% TFA) to afford the title compound (4 mg, 10%). ESI-MS m/z calcd for [C₁₆H₁₆Cl₂N₆O₄S] [M+H]⁺: 459.0; found: 459.0. ¹H NMR (500 MHz, Methanol-d₄) δ 8.65 (d, J=1.9 Hz, 1H), 8.48 (d, J=2.2 Hz, 1H), 8.32 (s, 1H), 8.25 (d, J=2.6 Hz, 1H), 8.20 (t, J=2.1 Hz, 1H), 6.51 (d, J=2.6 Hz, 1H), 5.93 (d, J=5.3 Hz, 1H), 5.01 (dd, J=11.4, 2.8 Hz, 1H), 4.92 (dd, J=11.4, 5.3 Hz, 1H), 4.47 (t, J=6.2 Hz, 1H), 4.22 (d, J=1.9 Hz, 1H), 3.77-3.65 (m, 2H).

### Example 6

### 5-Chloro-2-(trifluoromethyl)pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside

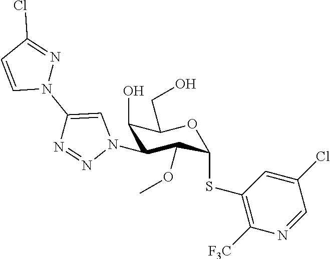

To a solution of 5-chloro-2-(trifluoromethyl)pyridin-3-yl 4,6-di-O-acetyl-3-azido-3-deoxy-2-O-methyl-1-thio-α-D-galactopyranoside (90 mg, 0.18 mmol), CuI (8.6 mg, 0.045 mmol) and 2-(3-chloropyrazol-1-yl)ethynyl(triisopropyl)silane (61 mg, 0.22 mmol) in MeCN (1.5 mL) DIPEA (93 μL, 0.54 mmol) was added followed by TBAF (45 μL, 1 M in THF, 0.045 mmol) and the mixture was stirred, first 5 h at 50° C. then overnight at rt. The mixture was concentrated and purified by chromatography (SiO₂, PE/EtOAc). The obtained material was stirred 1 h at rt in MeOH (1.5 mL) and NaOMe (0.3 mL, 1 M). The mixture was quenched with acetic acid (0.1 mL), concentrated, and purified by prep HPLC (C₁₈, H₂O/MeCN/0.2% NH₄OH) to afford the title compound (10.3 mg, 11%). ESI-MS m/z calcd for [C₁₈H₁₇C₁₂F₃N₆O₄S] [M+H]⁺: 541.0; found: 541.0. 1H NMR (400 MHz, Methanol-d₄) δ 8.55 (s, 2H), 8.37 (s, 1H), 8.26 (d, J=2.5 Hz, 1H), 6.52 (d, J=2.5 Hz, 1H), 6.38 (d, J=5.3 Hz, 1H), 5.04 (dd, J=11.3, 2.8 Hz, 1H), 4.68 (dd, J=11.4, 5.4 Hz, 1H), 4.42 (t, J=5.8 Hz, 1H), 4.21 (d, J=2.1 Hz, 1H), 3.72 (d, J=6.0 Hz, 2H), 3.41 (s, 3H).

### Example 7

### 5-Bromo-2-(trifluoromethyl)pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside

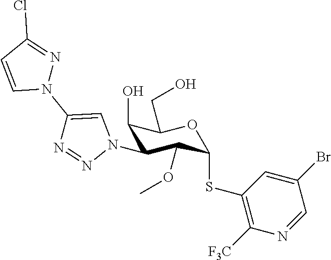

To a solution of 5-bromo-2-(trifluoromethyl)pyridin-3-yl 4,6-di-O-acetyl-3-azido-3-deoxy-2-O-methyl-1-thio-α-D-galactopyranoside (70 mg, 0.13 mmol), CuI (6.1 mg, 0.032 mmol) and 2-(3-chloropyrazol-1-yl)ethynyl(triisopropyl)silane (44 mg, 0.16 mmol) in MeCN (2 mL) DIPEA (66 μL, 0.39 mmol) was added followed by TBAF (32 μL, 1 M in THF, 0.032 mmol) and the mixture was stirred, first 5 h at 50° C. then overnight at rt. The mixture was concentrated and purified by chromatography (SiO₂, PE/EtOAc). The obtained material was stirred 30 min at rt in MeOH (1.5 mL) and NaOMe (0.3 mL, 1 M). The mixture was quenched with acetic acid (0.1 mL), concentrated, and purified by prep HPLC (C₁₈, H₂O/MeCN/0.2% NH₄OH) to afford the title compound (20.3 mg, 27%). ESI-MS m/z calcd for [C₁₈H₁₇BrClF₃N₆O₄S][M+H]⁺: 585.0; found: 585.0. 1H NMR (400 MHz, Methanol-d₄) δ 8.72-8.62 (m, 2H), 8.37 (s, 1H), 8.26 (d, J=2.6 Hz, 1H), 6.52 (d, J=2.6 Hz, 1H), 6.36 (d, J=5.3 Hz, 1H), 5.03 (dd, J=11.4, 2.8 Hz, 1H), 4.68 (dd, J=11.4, 5.4 Hz, 1H), 4.43 (t, J=6.1 Hz, 1H), 4.21 (d, J=2.3 Hz, 1H), 3.72 (d, J=6.0 Hz, 2H), 3.41 (s, 3H).

### Example 8

### 3-Chloro-2-(trifluoromethyl)pyridin-5-yl 3-[4-(3-chloro-1H-1,2-pyrazol-1-yl)-1H-1,2,3-triazol-1-yl]-3-deoxy-2-O-methyl-1-thio-α-D-galactopyranoside

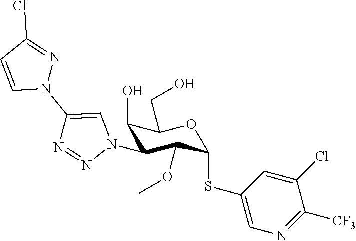

To a solution of 3-chloro-2-(trifluoromethyl)pyridin-5-yl 4,6-di-O-acetyl-3-azido-3-deoxy-2-O-methyl-1-thio-α-D-galactopyranoside (100 mg, 0.20 mmol), CuI (9.5 mg, 0.050 mmol) and 2-(3-chloropyrazol-1-yl)ethynyl(triisopropyl)silane (89 mg, 0.22 mmol) in MeCN (1.9 mL) DIPEA (103 μL, 0.60 mmol) was added followed by TBAF (220 μL, 1 M in THF, 0.22 mmol) and the mixture was stirred overnight at rt. The mixture was concentrated and purified by chromatography (SiO₂, PE/EtOAc). The obtained material was stirred 1 h at rt in MeOH (1.5 mL) and NaOMe (0.6 mL, 1 M). The mixture was quenched with acetic acid (0.1 mL), concentrated, and purified by prep HPLC (C₁₈, H₂O/MeCN/0.1% TFA) to afford the title compound (4.6 mg, 4%). ESI-MS m/z calcd for [C₁₈H₁₇Cl₂F₃N₆O₄S] [M+H]⁺: 541.0; found: 541.0. ¹H NMR (400 MHz, Methanol-d₄) δ 8.73 (d, J=1.8 Hz, 1H), 8.38 (s, 2H), 8.26 (d, J=2.6 Hz, 1H), 6.54-6.47 (m, 2H), 5.06 (dd, J=11.4, 2.8 Hz, 1H), 4.67 (dd, J=11.3, 5.3 Hz, 1H), 4.38 (t, J=5.8 Hz, 1H), 4.19 (s, 1H), 3.70 (d, J=6.1 Hz, 2H), 3.41 (s, 3H).

### Example 9

### 3-Bromo-2-(trifluoromethyl)pyridin-5-yl 3-[4-(3-chloro-1H-1,2-pyrazol-1-yl)-1H-1,2,3-triazol-1-yl]-3-deoxy-2-O-methyl-1-thio-α-D-galactopyranoside

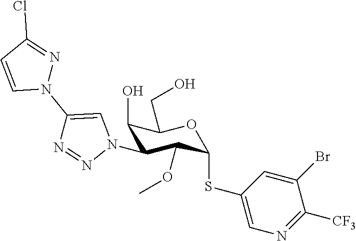

To a solution of 3-bromo-2-(trifluoromethyl)pyridin-5-yl 4,6-di-O-acetyl-3-azido-3-deoxy-2-O-methyl-1-thio-α-D-galactopyranoside (115 mg, 0.21 mmol), CuI (10 mg, 0.053 mmol) and 2-(3-chloropyrazol-1-yl)ethynyl(triisopropyl)silane (35 mg, 0.12 mmol) in MeCN (2 mL) DIPEA (59 μL, 0.34 mmol) was added followed by TBAF (53 μL, 1 M in THF, 0.53 mmol) and the mixture was stirred 2 days at rt. The mixture was concentrated and purified by chromatography (SiO₂, PE/EtOAc). The obtained material was stirred 1 h at rt in MeOH (1.5 mL) and NaOMe (0.3 mL, 1 M). The mixture was quenched with acetic acid (0.1 mL), concentrated, and purified by prep HPLC (C₁₈, H₂O/MeCN/0.1% TFA) to afford the title compound (4.3 mg, 6%). ESI-MS m/z calcd for [C₁₈H₁₇BrClF₃N₆O₄S] [M+H]⁺: 585.0; found: 585.0. ¹H NMR (400 MHz, Methanol-d₄) δ 8.77 (d, J=1.7 Hz, 1H), 8.54 (s, 1H), 8.38 (s, 1H), 8.26 (d, J=2.6 Hz, 1H), 6.52 (d, J=2.5 Hz, 1H), 6.49 (d, J=5.3 Hz, 1H), 5.06 (dd, J=11.3, 2.9 Hz, 1H), 4.67 (dd, J=11.3, 5.3 Hz, 1H), 4.39 (t, J=6.0 Hz, 1H), 4.20 (d, J=2.5 Hz, 1H), 3.73-3.64 (m, 2H), 3.41 (s, 3H).

### Example 10

### 5-Bromo-2-(N,N-dimethylcarbamoyl)pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside

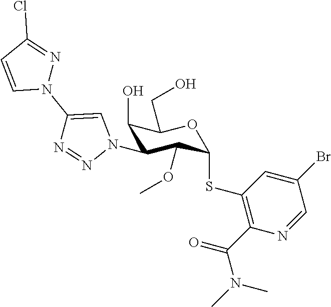

A solution of 5-bromo-2-carboxypyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside (24 mg, 0.04 mmol), HATU, (24 mg, 0.06 mmol), and dimethylamine (50 mg, 40% in H₂O, 0.44 mmol) in DMF (1 mL) was stirred 3 h rt. The mixture was quenched with HCl (1M) and extracted with EtOAc. The organic phase was washed with brine, dried, concentrated and purified by prep HPLC (C₁₈, H₂O/MeCN/0.1% TFA) to afford the title compound (2.8 mg, 11%). ESI-MS m/z calcd for [C₂₀H₂₃BrClN₇O₅S] [M+H]⁺: 588.0; found: 588.0. ¹H NMR (400 MHz, Methanol-d₄) δ 8.65 (d, J=1.9 Hz, 1H), 8.51 (d, J=1.9 Hz, 1H), 8.35 (s, 1H), 8.25 (d, J=2.5 Hz, 1H), 6.51 (d, J=2.5 Hz, 1H), 6.29 (d, J=5.2 Hz, 1H), 4.99 (dd, J=11.3, 2.7 Hz, 1H), 4.60 (dd, J=11.4, 5.3 Hz, 1H), 4.49 (t, J=6.0 Hz, 1H), 4.19 (d, J=2.5 Hz, 1H), 3.72 (d, J=5.8 Hz, 2H), 3.39 (s, 3H), 3.15 (s, 3H), 2.89 (s, 3H).

### Example 11

### 5-Bromo-2-cyanopyridin-3-yl 3-[4-(4-chloro-1H-1,2-pyrazol-1-yl)-1H-1,2,3-triazol-1-yl]-3-deoxy-2-O-methyl-1-thio-α-D-galactopyranoside

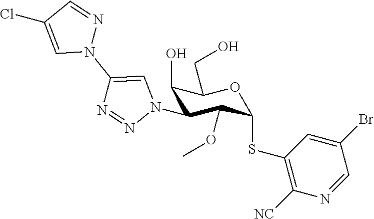

To a solution of 5-bromo-2-cyanopyridin-3-yl 3-azido-3-deoxy-2-O-methyl-1-thio-α-D-galactopyranoside (62 mg, 0.15 mmol), CuI (6 mg, 0.03 mmol) and 2-(4-chloropyrazol-1-yl)ethynyl(triisopropyl)silane (74 mg, 0.26 mmol) in MeCN (2 mL) triethylamine (84 μL, 0.6 mmol) was added followed by TBAF (15 μL, 1 M in THF, 0.015 mmol) and the mixture was stirred 5.5 h at 50° C. The mixture was cooled to rt and additional TBAF (150 μL, 1 M in THF, 0.15 mmol) was added. The mixture was stirred overnight at rt and was then filtered through a SCX-column (2 g, eluting with MeCN). The filtrate was concentrated and purified by prep HPLC (C₁₈, H₂O/MeCN/0.1% TFA) to afford the title compound (35 mg, 43%). ESI-MS m/z calcd for [C₁₈H₁₇BrClN₇O₄S] [M+H]⁺: 542.0; found: 541.8. ¹H NMR (400 MHz, Methanol-d₄) δ 8.71 (d, J=2.0 Hz, 1H), 8.62 (d, J=2.0 Hz, 1H), 8.40 (s, 1H), 8.35 (s, 1H), 7.74 (s, 1H), 6.50 (d, J=5.3 Hz, 1H), 5.08 (dd, J=11.3, 2.8 Hz, 1H), 4.70 (dd, J=11.3, 5.3 Hz, 1H), 4.40 (t, J=5.9 Hz, 1H), 4.21 (d, J=2.5 Hz, 1H), 3.68 (d, J=6.0 Hz, 2H), 3.46 (s, 3H).

### Example 12

### 5-Bromo-2-cyanopyridin-3-yl 3-deoxy-3-[4-(3-fluoro-1H-1,2-pyrazol-1-yl)-1H-1,2,3-triazol-1-yl]-2-O-methyl-1-thio-α-D-galactopyranoside

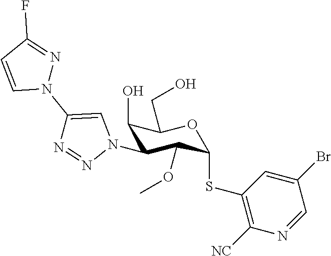

To a solution of 5-bromo-2-cyanopyridin-3-yl 3-azido-3-deoxy-2-O-methyl-1-thio-α-D-galactopyranoside (62 mg, 0.15 mmol), CuI (6 mg, 0.03 mmol) and 2-(3-fluoropyrazol-1-yl)ethynyl(triisopropyl)silane (91 mg, 0.23 mmol) in MeCN (2 mL) triethylamine (84 μL, 0.6 mmol) was added followed by TBAF (15 μL, 1 M in THF, 0.015 mmol) and the mixture was stirred 5.5 h at 50° C. The mixture was cooled to rt and additional TBAF (150 μL, 1 M in THF, 0.15 mmol) was added. The mixture was stirred overnight at rt and was then filtered through a SCX-column (2 g, eluting with MeCN). The filtrate was concentrated and purified by prep HPLC (C₁₈, H₂O/MeCN/0.1% TFA) to afford the title compound (10 mg, 13%). ESI-MS m/z calcd for [C₁₈H₁₇BrFN₇O₄S] [M+H]⁺: 526.0; found: 525.7. ¹H NMR (400 MHz, Methanol-d₄) δ 8.71 (d, J=2.0 Hz, 1H), 8.63 (d, J=2.0 Hz, 1H), 8.30 (s, 1H), 8.15 (t, J=2.6 Hz, 1H), 6.50 (d, J=5.3 Hz, 1H), 6.18 (dd, J=5.6, 2.7 Hz, 1H), 5.06 (dd, J=11.3, 2.9 Hz, 1H), 4.69 (dd, J=11.3, 5.3 Hz, 1H), 4.40 (t, J=6.0 Hz, 1H), 4.20 (d, J=2.3 Hz, 1H), 3.68 (d, J=6.0 Hz, 2H), 3.46 (s, 3H).

### Example 13

### 5-Bromo-2-cyanopyridin-3-yl 3-deoxy-3-[4-(4-fluoro-1H-1,2-pyrazol-1-yl)-1H-1,2,3-triazol-1-yl]-2-O-methyl-1-thio-α-D-galactopyranoside

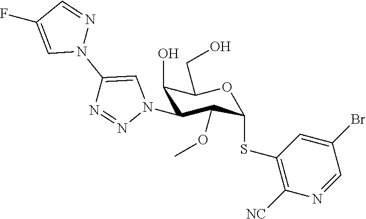

To a solution of 5-bromo-2-cyanopyridin-3-yl 3-azido-3-deoxy-2-O-methyl-1-thio-α-D-galactopyranoside (62 mg, 0.15 mmol), CuI (6 mg, 0.03 mmol) and 2-(4-fluoropyrazol-1-yl)ethynyl(triisopropyl)silane (122 mg, purity 49%, 0.23 mmol) in MeCN (2 mL) triethylamine (84 μL, 0.6 mmol) was added followed by TBAF (15 μL, 1 M in THF, 0.015 mmol) and the mixture was stirred 5.5 h at 50° C. The mixture was cooled to rt and additional TBAF (150 μL, 1 M in THF, 0.15 mmol) was added. The mixture was stirred overnight at rt and was then filtered through a SCX-column (2 g, eluting with MeCN). The filtrate was concentrated and purified by prep HPLC (C₁₈, H₂O/MeCN/0.1% TFA) to afford the title compound (40 mg, 50%). ESI-MS m/z calcd for [C₁₈H₁₇BrFN₇O₄S] [M+H]⁺: 526.0; found: 525.7. ¹H NMR (400 MHz, Methanol-d₄) δ 8.71 (d, J=2.0 Hz, 1H), 8.63 (d, J=2.0 Hz, 1H), 8.38 (s, 1H), 8.26 (d, J=4.4 Hz, 1H), 7.70 (d, J=3.8 Hz, 1H), 6.50 (d, J=5.3 Hz, 1H), 5.07 (dd, J=11.3, 2.9 Hz, 1H), 4.69 (dd, J=11.3, 5.3 Hz, 1H), 4.40 (t, J=6.0 Hz, 1H), 4.21 (d, J=2.7 Hz, 1H), 3.68 (d, J=6.0 Hz, 2H), 3.46 (s, 3H).

### Example 14

### 5-Bromo-2-cyanopyridin-3-yl 3-deoxy-3-[4-(3-methyl-1H-1,2-pyrazol-1-yl)-1H-1,2,3-triazol-1-yl]-2-O-methyl-1-thio-α-D-galactopyranoside

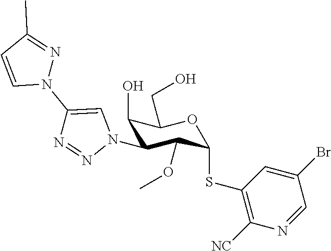

To a solution of 5-bromo-2-cyanopyridin-3-yl 3-azido-3-deoxy-2-O-methyl-1-thio-α-D-galactopyranoside (62 mg, 0.15 mmol), CuI (6 mg, 0.03 mmol) and 2-(3-methylpyrazol-1-yl)ethynyl(triisopropyl)silane (41 mg, 0.16 mmol) in MeCN (2 mL) triethylamine (84 μL, 0.6 mmol) was added followed by TBAF (15 μL, 1 M in THF, 0.015 mmol) and the mixture was stirred 5.5 h at 50° C. The mixture was cooled to rt and additional TBAF (150 μL, 1 M in THF, 0.15 mmol) was added. The mixture was stirred overnight at rt and was then filtered through a SCX-column (2 g, eluting with MeCN). The filtrate was concentrated and purified by prep HPLC (C₁₈, H₂O/MeCN/0.1% TFA) to afford the title compound (15 mg, 19%). ESI-MS m/z calcd for [C₁₉H₂₀BrN₇O₄S] [M+H]⁺: 522.1; found: 522.8. ¹H NMR (400 MHz, Methanol-d₄) δ 8.71 (d, J=2.0 Hz, 1H), 8.63 (d, J=2.1 Hz, 1H), 8.30 (s, 1H), 8.13 (d, J=2.5 Hz, 1H), 6.51 (d, J=5.3 Hz, 1H), 6.35 (d, J=2.3 Hz, 1H), 5.06 (dd, J=11.3, 2.8 Hz, 1H), 4.68 (dd, J=11.4, 5.3 Hz, 1H), 4.40 (t, J=5.8 Hz, 1H), 4.22 (d, J=2.6 Hz, 1H), 3.68 (d, J=6.0 Hz, 2H), 3.47 (s, 3H), 2.34 (s, 3H).

### Example 15

### 5-Bromo-2-cyanopyridin-3-yl 3-deoxy-3-[4-(5-methyl-1H-1,2-pyrazol-1-yl)-1H-1,2,3-triazol-1-yl]-2-O-methyl-1-thio-α-n-galactopyranoside

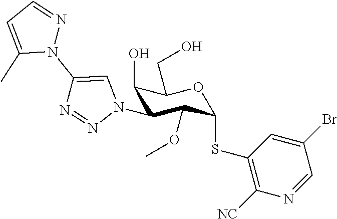

To a solution of 5-bromo-2-cyanopyridin-3-yl 3-azido-3-deoxy-2-O-methyl-1-thio-α-D-galactopyranoside (62 mg, 0.15 mmol), CuI (6 mg, 0.03 mmol) and 2-(5-methylpyrazol-1-yl)ethynyl(triisopropyl)silane (35 mg, 0.14 mmol) in MeCN (2 mL) triethylamine (84 μL, 0.6 mmol) was added followed by TBAF (15 μL, 1 M in THF, 0.015 mmol) and the mixture was stirred 5.5 h at 50° C. The mixture was cooled to rt and additional TBAF (150 μL, 1 M in THF, 0.15 mmol) was added. The mixture was stirred overnight at rt and was then filtered through a SCX-column (2 g, eluting with MeCN). The filtrate was concentrated and purified by prep HPLC (C₁₈, H₂O/MeCN/0.1% TFA). The obtained material was further purified by chromatography (SiO₂, PE/EtOAc) to afford the title compound (13 mg, 17%). ESI-MS m/z calcd for [C₁₉H₂₀BrN₇O₄S] [M+H]⁺: 522.1; found: 521.8. ¹H NMR (400 MHz, Methanol-d₄) δ 8.71 (d, J=2.0 Hz, 1H), 8.63 (d, J=2.0 Hz, 1H), 8.40 (s, 1H), 7.61 (d, J=1.6 Hz, 1H), 6.51 (d, J=5.3 Hz, 1H), 6.30 (s, 1H), 5.10 (dd, J=11.3, 2.9 Hz, 1H), 4.71 (dd, J=11.3, 5.3 Hz, 1H), 4.41 (t, J=5.9 Hz, 1H), 4.23 (d, J=2.4 Hz, 1H), 3.69 (d, J=6.0 Hz, 2H), 3.47 (s, 3H), 2.45 (s, 3H).

### Example 16

### 5-Bromo-2-cyanopyridin-3-yl 3-[4-(3-chloro-5-methyl-1H-1,2-pyrazol-1-yl)-1H-1,2,3-triazol-1-yl]-3-deoxy-2-O-methyl-1-thio-α-D-galactopyranoside

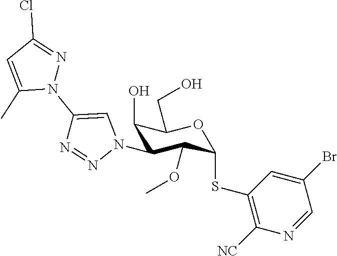

To a solution of 5-bromo-2-cyanopyridin-3-yl 3-azido-3-deoxy-2-O-methyl-1-thio-α-D-galactopyranoside (70 mg, 0.17 mmol), CuI (6 mg, 0.03 mmol) and 2-(3-chloro-5-methylpyrazol-1-yl)ethynyl(triisopropyl)silane (60 mg, 0.20 mmol) in MeCN (1 mL) triethylamine (94 μL, 0.67 mmol) was added followed by TBAF (168 μL, 1 M in THF, 0.17 mmol). Acetic acid (9.6 μL, 0.17 mmol) was added and the mixture was stirred 1 h at rt and then filtered through a SCX-column (2 g, eluting with MeCN). The filtrate was concentrated and purified by prep HPLC (C₁₈, H₂O/MeCN/0.1% formic acid) to afford the title compound (8 mg, 9%). ESI-MS m/z calcd for [C₁₉H₁₉BrClN₇O₄S][M+H]⁺: 556.0; found: 555.8. ¹H NMR (400 MHz, Methanol-d₄) δ 8.71 (d, J=2.0 Hz, 1H), 8.63 (d, J=2.0 Hz, 1H), 8.41 (s, 1H), 6.51 (d, J=5.3 Hz, 1H), 6.29 (s, 1H), 5.09 (dd, J=11.3, 2.9 Hz, 1H), 4.70 (dd, J=11.3, 5.3 Hz, 1H), 4.41 (t, J=6.0 Hz, 1H), 4.22 (d, J=2.5 Hz, 1H), 3.69 (d, J=6.0 Hz, 2H), 3.47 (s, 3H), 2.44 (s, 3H).

### Example 17

### 5-Bromo-2-cyanopyridin-3-yl 3-{4-[5-chloro-3-(trifluoromethyl)-1H-1,2-pyrazol-1-yl]-1H-1,2,3-triazol-1-yl}-3-deoxy-2-O-methyl-1-thio-α-D-galactopyranoside

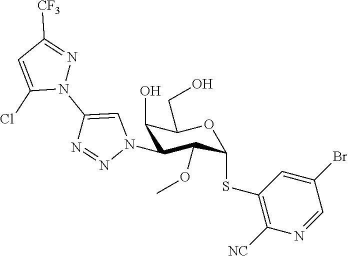

To a solution of 5-bromo-2-cyanopyridin-3-yl 3-azido-3-deoxy-2-O-methyl-1-thio-α-D-galactopyranoside (70 mg, 0.17 mmol), CuI (6 mg, 0.03 mmol) and 2-(3-chloro-5-methylpyrazol-1-yl)ethynyl(triisopropyl)silane (80 mg, 0.23 mmol) in MeCN (1 mL) triethylamine (94 μL, 0.67 mmol) was added followed by TBAF (168 μL, 1 M in THF, 0.17 mmol). Acetic acid (9.6 μL, 0.17 mmol) was added and the mixture was stirred 1 h at rt and then filtered through a SCX-column (2 g, eluting with MeCN). The filtrate was concentrated and purified by prep HPLC (C₁₈, H₂O/MeCN/0.1% formic acid) to afford the title compound (10 mg, 10%). ESI-MS m/z calcd for [C₁₉H₁₆BrClF₃N₇O₄S][M+H]⁺: 610.0; found: 609.7. ¹H NMR (500 MHz, Methanol-d₄) δ 8.71 (d, J=2.0 Hz, 1H), 8.65 (s, 1H), 8.63 (d, J=2.0 Hz, 1H), 6.98 (s, 1H), 6.50 (d, J=5.3 Hz, 1H), 5.15 (dd, J=11.3, 2.9 Hz, 1H), 4.72 (dd, J=11.3, 5.3 Hz, 1H), 4.42 (t, J=6.0 Hz, 1H), 4.25 (d, J=2.5 Hz, 1H), 3.69 (d, J=6.0 Hz, 2H), 3.48 (s, 3H).

### Example 18

### 5-Bromo-2-cyanopyridin-3-yl 3-[4-(3-chloro-4-methyl-1H-1,2-pyrazol-1-yl)-1H-1,2,3-triazol-1-yl]-3-deoxy-2-O-methyl-1-thio-α-D-galactopyranoside

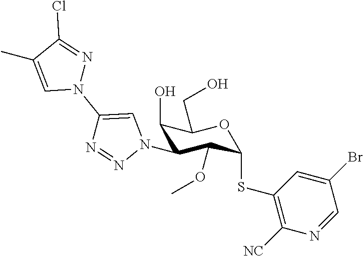

To a solution of 5-bromo-2-cyanopyridin-3-yl 3-azido-3-deoxy-2-O-methyl-1-thio-α-D-galactopyranoside (62 mg, 0.15 mmol), CuI (6 mg, 0.03 mmol) and 3-chloro-1-ethynyl-4-methylpyrazole (48 mg, 0.23 mmol) in MeCN (2 mL) triethylamine (84 μL, 0.6 mmol) was added and the mixture was stirred 1 h at 50° C. The mixture was cooled to rt, concentrated, and purified by prep HPLC (C₁₈, H₂O/MeCN/0.1% TFA). The obtained material was further purified by chromatography (SiO₂, PE/EtOAc) to afford the title compound (24.2 mg, 29%). ESI-MS m/z calcd for [C₁₉H₁₉BrClN₇O₄S][M+H]⁺: 556.0; found: 555.7. ¹H NMR (400 MHz, Methanol-d₄) δ 8.71 (d, J=1.8 Hz, 1H), 8.63 (d, J=1.8 Hz, 1H), 8.32 (s, 1H), 8.09 (s, 1H), 6.50 (d, J=5.2 Hz, 1H), 5.06 (dd, J=11.3, 2.7 Hz, 1H), 4.69 (dd, J=11.3, 5.3 Hz, 1H), 4.40 (t, J=5.9 Hz, 1H), 4.24-4.16 (m, 1H), 3.68 (d, J=6.0 Hz, 2H), 3.46 (s, 3H), 2.12 (s, 3H).

### Example 19

### 5-Bromo-2-(N,N-ethylisopropylcarbamoyl)pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside

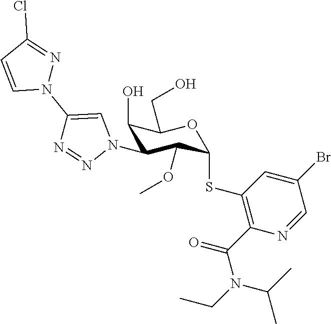

A solution of 5-bromo-2-carboxypyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside (70 mg, 0.13 mmol), HATU (71 mg, 0.06 mmol), and N-ethylisopropylamine (22 mg, 0.25 mmol) in DMF (1 mL) was stirred overnight at rt. The mixture was heated to 50° C. and stirred overnight. The mixture was quenched with HCl (1 M) and extracted with EtOAc. The organic phase was washed with brine, dried, concentrated and purified by prep HPLC (Cis, H₂O/MeCN/0.1% TFA) to afford the title compound (6.3 mg, 8%). ESI-MS m/z calcd for [C₂₃H₂₉BrClN₇O₅S] [M+H]⁺: 630.1; found: 630.1. ¹H NMR (500 MHz, Methanol-d₄) δ 8.67-8.62 (m, 1H), 8.56-8.51 (m, 1H), 8.39-8.35 (m, 1H), 8.28-8.22 (m, 1H), 6.55-6.50 (m, 1H), 6.33-6.27 (m, 1H), 5.04-4.97 (m, 1H), 4.66-4.59 (m, 1H), 4.57-4.49 (m, 1H), 4.21 (s, 1H), 3.81-3.70 (m, 2H), 3.64-3.48 (m, 2H), 3.41 (s, 3H), 3.28-3.13 (m, 1H), 1.44-1.08 (m, 9H).

### Example 20

### 5-Bromo-2-(N,N-diisobutylcarbamoyl)pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside

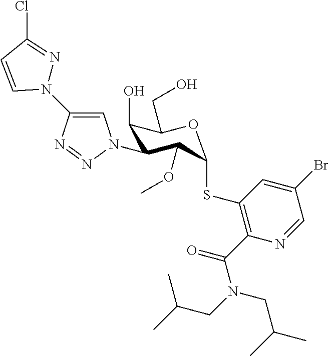

To a solution of 5-bromo-2-carboxypyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside (18 mg, 0.032 mmol), triethylamine (9 μL, 0.64 mmol) and diisobutylamine (11 μL, 0.064 mmol) in MeCN (0.5 mL) HATU (13 mg, 0.035 mmol) was added and the mixture was stirred 30 min at rt. The mixture was diluted with MeOH/H₂O and purified by prep HPLC (C₁₈, H₂O/MeCN/0.1% TFA) to afford the title compound (10.2 mg, 47%). ESI-MS m/z calcd for [C₂₆H₃₅BrClN₇O₅S] [M+H]⁺: 672.1; found: 672.0. ¹H NMR (400 MHz, Methanol-d₄) δ 8.62 (d, J=2.1 Hz, 1H), 8.53 (d, J=2.0 Hz, 1H), 8.36 (s, 1H), 8.25 (d, J=2.6 Hz, 1H), 6.51 (d, J=2.5 Hz, 1H), 6.33 (d, J=5.2 Hz, 1H), 4.97 (dd, J=11.4, 2.8 Hz, 1H), 4.60 (dd, J=11.3, 5.3 Hz, 1H), 4.49 (t, J=5.9 Hz, 1H), 4.18 (d, J=2.6 Hz, 1H), 3.72 (d, J=6.0 Hz, 2H), 3.47 (dd, J=13.5, 7.8 Hz, 1H), 3.40-3.35 (m, 4H), 3.03 (dd, J=7.5, 2.4 Hz, 2H), 2.21 (dt, J=13.7, 6.8 Hz, 1H), 1.94-1.82 (m, 1H), 1.04 (dd, J=6.6, 3.5 Hz, 6H), 0.82 (t, J=6.5 Hz, 6H).

### Example 21

### 5-Bromo-2-[N,N-(cyclopropylmethyl)ethylcarbamoyl]pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside

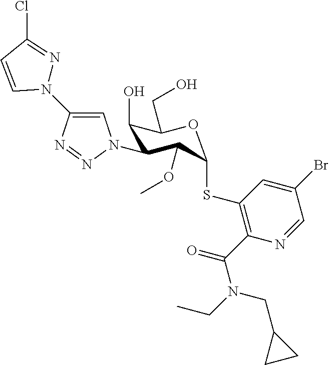

To a solution of 5-bromo-2-carboxypyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside (18 mg, 0.032 mmol), 4-methylmorpholine (14 μL, 0.13 mmol) and N-(cyclopropylmethyl)ethanamine (6.4 mg, 0.064 mmol) in MeCN (0.5 mL) HATU (13 mg, 0.035 mmol) was added and the mixture was stirred 1 h at rt. The mixture was diluted with MeOH/H₂O and purified by prep HPLC (C₁₈, H₂O/MeCN/0.1% TFA) to afford the title compound (12.7 mg, 62%). ESI-MS m/z calcd for [C₂₄H₂₉BrClN₇O₅S][M+H]⁺: 642.1; found: 642.0. ¹H NMR (400 MHz, Methanol-d₄) δ 8.66-8.61 (m, 1H), 8.56-8.49 (m, 1H), 8.35 (s, 1H), 8.25 (d, J=2.6 Hz, 1H), 6.51 (d, J=2.6 Hz, 1H), 6.35-6.23 (m, 1H), 5.03-4.94 (m, 1H), 4.60 (dd, J=11.3, 5.4 Hz, 1H), 4.55-4.46 (m, 1H), 4.18 (d, J=2.6 Hz, 1H), 3.76-3.00 (m, 9H), 1.38-0.97 (m, 4H), 0.67-0.08 (m, 4H).

### Example 22

### 5-Bromo-2-[N,N-(2-fluoro-2-methylpropyl)methylcarbamoyl]pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside

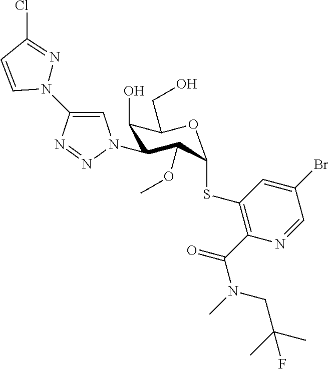

To a solution of 5-bromo-2-carboxypyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside (25 mg, 0.045 mmol), 4-methylmorpholine (20 μL, 0.18 mmol) and (2-fluoro-2-methylpropyl)(methyl)amine hydrochloride (12.6 mg, 0.089 mmol) in MeCN (0.5 mL) HATU (19 mg, 0.049 mmol) was added and the mixture was stirred 1 h at rt. The mixture was diluted with MeOH/H₂O and purified by prep HPLC (C₁₈, H₂O/MeCN/0.1% TFA) to afford the title compound (19.7 mg, 68%). ESI-MS m/z calcd for [C₂₃H₂₅BrFClN₇O₅S] [M+H]⁺: 648.1; found: 648.0. ¹H NMR (400 MHz, Methanol-d₄,) δ 8.66 (d, J=2.0 Hz, 1H), 8.57 (d, J=2.0 Hz, 1H), 8.35 (s, 1H), 8.25 (d, J=2.6 Hz, 1H), 6.51 (d, J=2.6 Hz, 1H), 6.31 (d, J=5.2 Hz, 1H), 4.98 (dd, J=11.3, 3.0 Hz, 1H), 4.61 (dd, J=11.4, 5.3 Hz, 1H), 4.50 (t, J=6.0 Hz, 1H), 4.19 (s, 1H), 3.94-3.64 (m, 4H), 3.37 (s, 3H), 2.95 (s, 3H), 1.47 (d, J=21.4 Hz, 6H).

### Example 23

### 5-Bromo-2-[N,N-(tert-butyl)ethylcarbamoyl]pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside

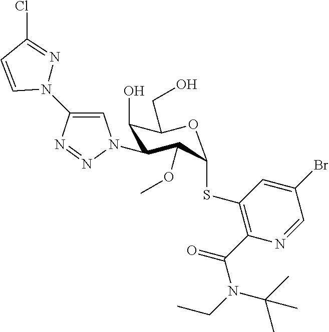

To a solution of 5-bromo-2-carboxypyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside (25 mg, 0.045 mmol), 4-methylmorpholine (20 μL, 0.18 mmol) and N-tert-butylmethylamine (9.0 mg, 0.089 mmol) in MeCN (0.5 mL) HATU (19 mg, 0.049 mmol) was added and the mixture was stirred 1 h at rt. The mixture was diluted with MeOH/H₂O and purified by prep HPLC (C₁₈, H₂O/MeCN/0.1% TFA) to afford the title compound (5.6 mg, 20%). ESI-MS m/z calcd for [C₂₄H₃₁BrClN₇O₅S] [M+H]⁺: 644.1; found: 644.0. ¹H NMR (400 MHz, Methanol-d₄) δ 8.59 (d, J=2.0 Hz, 1H), 8.54 (d, J=2.0 Hz, 1H), 8.36 (s, 1H), 8.25 (d, J=2.5 Hz, 1H), 6.51 (d, J=2.5 Hz, 1H), 6.27 (d, J=5.2 Hz, 1H), 4.98 (dd, J=11.4, 2.9 Hz, 1H), 4.61 (dd, J=11.5, 5.3 Hz, 1H), 4.52 (t, J=6.0 Hz, 1H), 4.19 (d, J=2.4 Hz, 1H), 3.78-3.72 (m, 2H), 3.38 (s, 3H), 3.29-3.21 (m, 2H), 1.61 (s, 9H), 1.13 (t, J=7.0 Hz, 3H).

### Example 24

### 5-Bromo-2-[N,N-bis(cyclopropylmethyl)carbamoyl]pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside

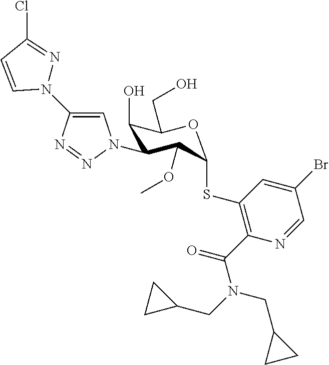

To a solution of 5-bromo-2-carboxypyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside (25 mg, 0.045 mmol), 4-methylmorpholine (20 μL, 0.18 mmol) and bis(cyclopropylmethyl)amine hydrochloride (14.4 mg, 0.089 mmol) in MeCN (0.5 mL) HATU (19 mg, 0.049 mmol) was added and the mixture was stirred 1 h at rt. The mixture was diluted with MeOH/H₂O and purified by prep HPLC (C₁₈, H₂O/MeCN/0.1% TFA) to afford the title compound (13.9 mg, 47%). ESI-MS m/z calcd for [C₂₆H₃₁BrClN₇O₅S] [M+H]⁺: 668.1; found: 668.0. ¹H NMR (400 MHz, Methanol-d₄) δ 8.62 (d, J=2.0 Hz, 1H), 8.53 (d, J=2.0 Hz, 1H), 8.35 (s, 1H), 8.24 (d, J=2.6 Hz, 1H), 6.51 (d, J=2.6 Hz, 1H), 6.31 (d, J=5.3 Hz, 1H), 4.98 (dd, J=11.3, 2.8 Hz, 1H), 4.60 (dd, J=11.4, 5.3 Hz, 1H), 4.49 (t, J=5.9 Hz, 1H), 4.18 (d, J=2.4 Hz, 1H), 3.72 (d, J=6.0 Hz, 2H), 3.67-3.52 (m, 2H), 3.38 (s, 3H), 3.13 (d, J=6.7 Hz, 2H), 1.31-1.20 (m, 1H), 1.10-1.00 (m, 1H), 0.65-0.05 (m, 8H).

### Example 25

### 5-Bromo-2-[N,N-(cyclobutylmethyl)ethylcarbamoyl}pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside

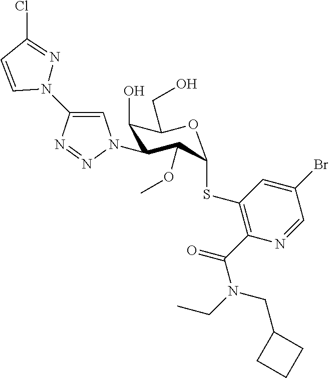

To a solution of 5-bromo-2-carboxypyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside (25 mg, 0.045 mmol), 4-methylmorpholine (20 μL, 0.18 mmol) and (cyclobutylmethyl)(ethyl)amine hydrochloride (13.3 mg, 0.089 mmol) in MeCN (0.5 mL) HATU (19 mg, 0.049 mmol) was added and the mixture was stirred 1 h at rt. The mixture was diluted with MeOH/H₂O and purified by prep HPLC (C₁₈, H₂O/MeCN/0.1% TFA) to afford the title compound (13.0 mg, 45%). ESI-MS m/z calcd for [C₂₅H₃₁BrClN₇O₅S] [M+H]⁺: 656.1; found: 656.0. ¹H NMR (400 MHz, Methanol-d₄) δ 8.67-8.59 (m, 1H), 8.56-8.50 (m, 1H), 8.36 (s, 1H), 8.25 (d, J=2.5 Hz, 1H), 6.51 (d, J=2.5 Hz, 1H), 6.35-6.24 (m, 1H), 5.03-4.94 (m, 1H), 4.65-4.56 (m, 1H), 4.55-4.44 (m, 1H), 4.22-4.16 (m, 1H), 3.76-3.69 (m, 2H), 3.64-3.53 (m, 2H), 3.42-3.37 (m, 3H), 3.25-3.09 (m, 2H), 2.89-2.56 (m, 1H), 2.22-1.56 (m, 6H), 1.32-1.05 (m, 3H).

### Example 26

### 5-Bromo-2-[N,N-(cyclobutylmethyl)isopropylcarbamoyl]pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside

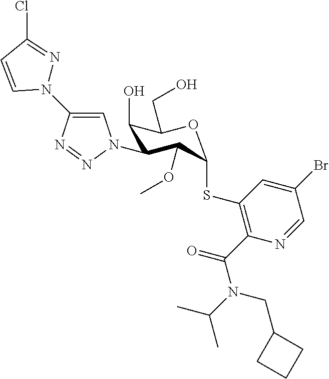

To a solution of 5-bromo-2-carboxypyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside (25 mg, 0.045 mmol), 4-methylmorpholine (20 μL, 0.18 mmol) and (cyclobutylmethyl)(isopropanyl)amine hydrochloride (14.6 mg, 0.089 mmol) in MeCN (0.5 mL) HATU (19 mg, 0.049 mmol) was added and the mixture was stirred 1 h at rt. The mixture was diluted with MeOH/H₂O and purified by prep HPLC (C₁₈, H₂O/MeCN/0.1% TFA) to afford the title compound (11.8 mg, 40%). ESI-MS m/z calcd for [C₂₆H₃₃BrClN₇O₅S] [M+H]⁺: 670.1; found: 670.0. ¹H NMR (400 MHz, Methanol-d₄) δ 8.67-8.58 (m, 1H), 8.55-8.49 (m, 1H), 8.39-8.34 (m, 1H), 8.25 (d, J=2.5 Hz, 1H), 6.51 (d, J=2.6 Hz, 1H), 6.35-6.23 (m, 1H), 5.03-4.95 (m, 1H), 4.66-4.56 (m, 1H), 4.56-4.44 (m, 1H), 4.38-3.51 (m, 4H), 3.50-3.08 (m, 5H), 2.91-2.49 (m, 1H), 2.20-1.45 (m, 6H), 1.42-1.13 (m, 6H).

### Example 27

### 5-Bromo-2-[N,N-bis(cyclobutylmethyl)carbamoyl]pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside

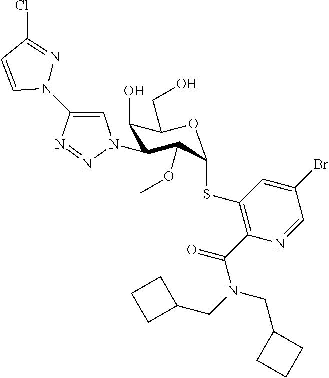

To a solution of 5-bromo-2-carboxypyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside (25 mg, 0.045 mmol), 4-methylmorpholine (20 μL, 0.18 mmol) and bis(cyclobutylmethyl)amine hydrochloride (16.9 mg, 0.089 mmol) in MeCN (0.5 mL) HATU (19 mg, 0.049 mmol) was added and the mixture was stirred 1 h at rt. The mixture was diluted with MeOH/H₂O and purified by prep HPLC (C₁₈, H₂O/MeCN/0.1% TFA) to afford the title compound (11.8 mg, 40%). ESI-MS m/z calcd for [C₂₈H₃₅BrClN₇O₅S] [M+H]⁺: 696.1; found: 696.0. ¹H NMR (400 MHz, Methanol-d₄) δ 8.62 (d, J=2.0 Hz, 1H), 8.53 (d, J=2.0 Hz, 1H), 8.36 (s, 1H), 8.25 (d, J=2.5 Hz, 1H), 6.51 (d, J=2.5 Hz, 1H), 6.33 (d, J=5.2 Hz, 1H), 4.99 (dd, J=11.4, 2.8 Hz, 1H), 4.61 (dd, J=11.4, 5.3 Hz, 1H), 4.48 (t, J=5.9 Hz, 1H), 4.19 (d, J=2.3 Hz, 1H), 3.72 (d, J=6.0 Hz, 2H), 3.66-3.52 (m, 2H), 3.40 (s, 3H), 3.15 (dd, J=7.2, 2.9 Hz, 2H), 2.81 (dt, J=14.5, 7.3 Hz, 1H), 2.61 (p, J=7.6 Hz, 1H), 2.22-1.51 (m, 12H).

### Example 28

### 5-Bromo-2-(pyrrolidine-1-carbonyl)pyridin-3-yl 3-[4-(3-chloro-1H-1,2-pyrazol-1-yl)-1H-1,2,3-triazol-1-yl ]-3-deoxy-2-O-methyl-1-thio-α-D-galactopyranoside

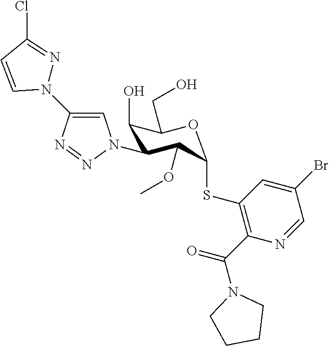

A solution of 5-bromo-2-carboxypyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside (20 mg, 0.036 mmol), DIPEA (18 μL, 0.11 mmol), pyrrolidine (6 μL, 0.071 mmol) and benzotriazol-1-yloxytripyrrolidinophosphonium hexaphosphate (20.4 mg, 0.039 mmol) in MeCN (0.5 mL) was stirred overnight at rt. The mixture was diluted with H₂O and purified by prep HPLC (C₁₈, H₂O/MeCN/0.2% ammonium hydroxide) to afford the title compound (12 mg, 55%). ESI-MS m/z calcd for [C₂₂H₂₅BrClN₇O₅S] [M+H]⁺: 614.1; found: 614.1. ¹H NMR (400 MHz, Methanol-d₄) δ 8.64 (d, J=2.0 Hz, 1H), 8.52 (d, J=2.1 Hz, 1H), 8.36 (s, 1H), 8.25 (d, J=2.6 Hz, 1H), 6.51 (d, J=2.6 Hz, 1H), 6.32 (d, J=5.3 Hz, 1H), 5.00 (dd, J=11.3, 3.0 Hz, 1H), 4.61 (dd, J=11.4, 5.3 Hz, 1H), 4.49 (t, J=6.0 Hz, 1H), 4.18 (d, J=2.3 Hz, 1H), 3.71 (d, J=6.0 Hz, 2H), 3.64 (t, J=6.7 Hz, 2H), 3.39 (s, 3H), 3.27 (t, J=5.9 Hz, 2H), 2.11-1.86 (m, 4H).

### Example 29

### 5-Bromo-2-[N,N-ethyl-(2,2,2-trifluoroethyl)carbamoyl]pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside

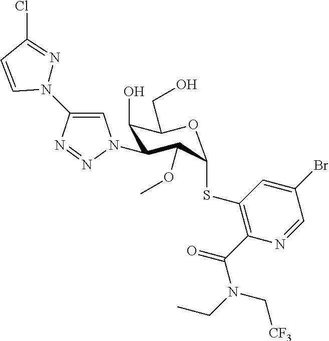

To a solution of 5-bromo-2-carboxypyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside (25 mg, 0.045 mmol), 4-methylmorpholine (20 μL, 0.18 mmol) and N-ethyl-2,2,2-trifluoroethanamine hydrochloride (14.6 mg, 0.089 mmol) in MeCN (0.5 mL) HATU (19 mg, 0.049 mmol) was added and the mixture was stirred 1 h at rt. The mixture was diluted with MeOH/H₂O and purified by prep HPLC (C₁₈, H₂O/MeCN/0.1% TFA) to afford the title compound (14.5 mg, 49%). ESI-MS m/z calcd for [C₂₂H₂₄BrF₃ClN₇O₅S][M+H]⁺: 670.1; found: 670.0. ¹H NMR (500 MHz, Methanol-d₄) δ 8.69-8.65 (m, 1H), 8.59-8.56 (m, 1H), 8.38-8.35 (m, 1H), 8.26 (d, J=2.6 Hz, 1H), 6.53 (d, J=2.6 Hz, 1H), 6.35-6.26 (m, 1H), 5.04-4.97 (m, 1H), 4.66-4.59 (m, 1H), 4.55-4.49 (m, 1H), 4.40-4.07 (m, 3H), 3.81-3.35 (m, 7H), 1.37-1.12 (m, 3H).

### Example 30

### 5-Bromo-2-[N,N-ethyl(2-fluoro-2-methylpropyl)carbamoyl]pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside

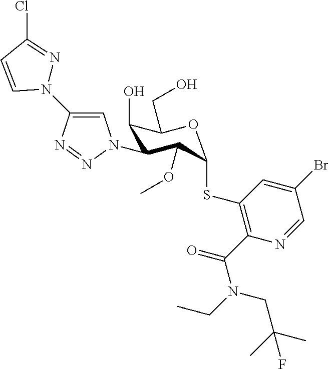

To a solution of 5-bromo-2-carboxypyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside (25 mg, 0.045 mmol), 4-methylmorpholine (20 μL, 0.18 mmol) and (ethyl)(2-fluoro-2-methylpropyl) amine hydrochloride (13.9 mg, 0.089 mmol) in MeCN (0.5 mL) HATU (19 mg, 0.049 mmol) was added and the mixture was stirred 1 h at rt. The mixture was diluted with MeOH/H₂O and purified by prep HPLC (C₁₈, H₂O/MeCN/0.1% TFA) to afford the title compound (14.1 mg, 48%). ESI-MS m/z calcd for [C₂₄H₃₀BrFClN₇O₅S][M+H]⁺: 662.1; found: 662.0. ¹H NMR (500 MHz, Methanol-d₄) δ 8.68-8.61 (m, 1H), 8.61-8.50 (m, 1H), 8.39-8.35 (m, 1H), 8.27 (d, J=2.6 Hz, 1H), 6.53 (d, J=2.6 Hz, 1H), 6.36-6.27 (m, 1H), 5.04-4.96 (m, 1H), 4.62 (dd, J=11.4, 5.3 Hz, 1H), 4.52 (t, J=6.0 Hz, 1H), 4.24-4.17 (m, 1H), 3.93-3.69 (m, 4H), 3.44-3.35 (m, 5H), 1.56-1.11 (m, 9H).

### Example 31

### 5-Bromo-2-[N,N-(cyclopropylmethyl)isopropylcarbamoyl]pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside

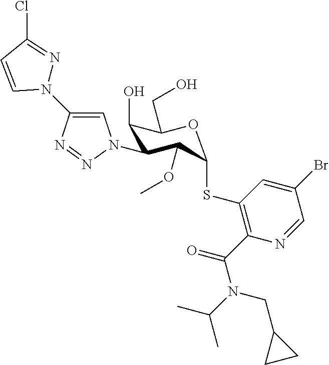

To a solution of 5-bromo-2-carboxypyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside (25 mg, 0.045 mmol), 4-methylmorpholine (20 μL, 0.18 mmol) and N-(cyclopropylmethyl)-2-propanamine hydrochloride (13.3 mg, 0.089 mmol) in MeCN (0.5 mL) HATU (19 mg, 0.049 mmol) was added and the mixture was stirred 1 h at rt. The mixture was diluted with MeOH/H₂O and purified by prep HPLC (Cis, H₂O/MeCN/0.1% TFA) to afford the title compound (13.6 mg, 47%). ESI-MS m/z calcd for [C₂₅H₃₁BrClN₇O₅S][M+H]⁺: 656.1; found: 656.0. ¹H NMR (500 MHz, Methanol-d₄) δ 8.67-8.62 (m, 1H), 8.55-8.51 (m, 1H), 8.37 (s, 1H), 8.28-8.23 (m, 1H), 6.53 (d, J=2.6 Hz, 1H), 6.36-6.26 (m, 1H), 5.05-4.96 (m, 1H), 4.66-4.58 (m, 1H), 4.57-3.59 (m, 5H), 3.44-3.02 (m, 5H), 1.50-0.06 (m, 11H).

### Example 32

### 5-Bromo-2-(N,N-isobutylisopropylcarbamoyl)pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside

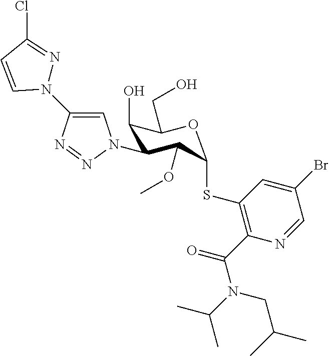

To a solution of 5-bromo-2-carboxypyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside (25 mg, 0.045 mmol), 4-methylmorpholine (20 μL, 0.18 mmol) and N-isopropyl-2-methylpropan-1-amine hydrochloride (13.5 mg, 0.089 mmol) in MeCN (0.5 mL) HATU (19 mg, 0.049 mmol) was added and the mixture was stirred 1 h at rt. The mixture was diluted with MeOH/H₂O and purified by prep HPLC (C₁₈, H₂O/MeCN/0.1% TFA) to afford the title compound (10.7 mg, 37%). ESI-MS m/z calcd for [C₂₅H₃₃BrClN₇O₅S] [M+H]⁺: 658.1; found: 658.0. ¹H NMR (500 MHz, Methanol-d₄) δ 8.66-8.61 (m, 1H), 8.57-8.50 (m, 1H), 8.40-8.35 (m, 1H), 8.26 (d, J=2.6 Hz, 1H), 6.53 (d, J=2.6 Hz, 1H), 6.36-6.27 (m, 1H), 5.03-4.96 (m, 1H), 4.62 (dd, J=11.4, 5.3 Hz, 1H), 4.56-4.47 (m, 1H), 4.27-3.61 (m, 4H), 3.43-3.37 (m, 3H), 3.38-2.92 (m, 2H), 2.39-1.78 (m, 1H), 1.53-1.19 (m, 6H), 1.09-0.80 (m, 6H).

### Example 33

### 5-Bromo-2-[N,N-(cyclopropylmethyl)methoxycarbamoyl]pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside

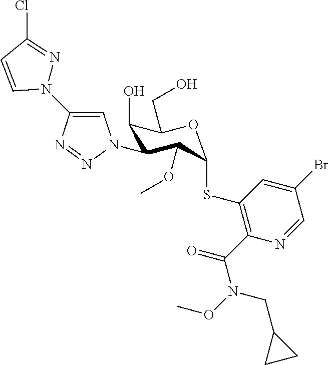

To a solution of 5-bromo-2-carboxypyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside (25 mg, 0.045 mmol), 4-methylmorpholine (20 μL, 0.18 mmol) and (cyclopropylmethyl)(methoxy)amine hydrochloride (12.2 mg, 0.089 mmol) in MeCN (0.5 mL) HATU (19 mg, 0.049 mmol) was added and the mixture was stirred 1 h at rt. The mixture was diluted with MeOH/H₂O and purified by prep HPLC (C₁₈, H₂O/MeCN/0.1% TFA) to afford the title compound (17.5 mg, 61%). ESI-MS m/z calcd for [C₂₃H₂₇BrClN₇O₆S] [M+H]⁺: 644.1; found: 644.0. ¹H NMR (400 MHz, Methanol-d₄) δ 8.64 (d, J=2.0 Hz, 1H), 8.57-8.49 (m, 1H), 8.38-8.33 (m, 1H), 8.25 (d, J=2.6 Hz, 1H), 6.51 (d, J=2.6 Hz, 1H), 6.34-6.20 (m, 1H), 4.99 (dd, J=11.3, 2.9 Hz, 1H), 4.59 (dd, J=11.3, 5.3 Hz, 1H), 4.51 (t, J=5.9 Hz, 1H), 4.18 (d, J=2.3 Hz, 1H), 4.01-3.56 (m, 7H), 3.43-3.36 (m, 3H), 1.36-1.09 (m, 1H), 0.71-0.52 (m, 2H), 0.48-0.16 (m, 2H).

### Example 34

### 5-Bromo-2-(N-methylcarbamoyl)pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside

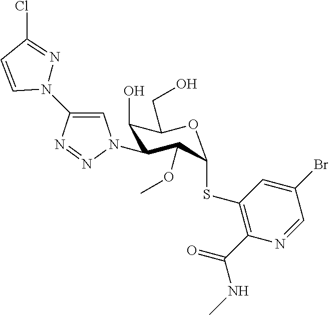

To a solution of 5-bromo-2-carboxypyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside (25 mg, 0.045 mmol), 4-methylmorpholine (20 μL, 0.18 mmol), methanesulfonic acid (8.7 μL, 0.13 mmol) and methylamine (44.5 μL, 2M in THF, 0.089 mmol) in MeCN (0.5 mL) HATU (19 mg, 0.049 mmol) was added and the mixture was stirred 1 h at rt. The mixture was diluted with MeOH/H₂O and purified by prep HPLC (C₁₈, H₂O/MeCN/0.1% TFA) to afford the title compound (17.2 mg, 67%). ESI-MS m/z calcd for [C₁₉H₂₁BrClN₇O₅S] [M+H]⁺: 574.0; found: 574.0. ¹H NMR (400 MHz, Methanol-d₄) δ 8.53 (d, J=1.6 Hz, 1H), 8.50 (d, J=1.5 Hz, 1H), 8.36 (s, 1H), 8.26 (d, J=2.5 Hz, 1H), 6.52 (d, J=2.5 Hz, 1H), 6.39 (d, J=5.4 Hz, 1H), 5.11 (dd, J=11.4, 2.8 Hz, 1H), 4.67 (dd, J=11.4, 5.4 Hz, 1H), 4.36 (t, J=6.0 Hz, 1H), 4.21-4.16 (m, 1H), 3.71 (dd, J=11.4, 5.4 Hz, 1H), 3.65 (dd, J=11.4, 6.9 Hz, 1H), 3.38 (s, 3H), 2.93 (s, 3H).

### Example 35

### 5-Bromo-2-(N-ethylcarbamoyl)pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside

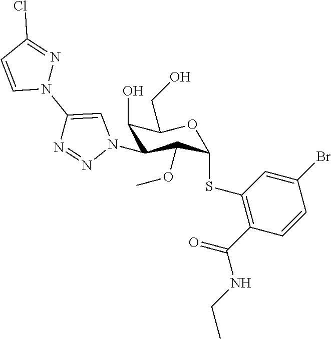

To a solution of 5-bromo-2-carboxypyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside (25 mg, 0.045 mmol), 4-methylmorpholine (20 μL, 0.18 mmol), methanesulfonic acid (8.7 μL, 0.13 mmol) and ethylamine hydrochloride (7.3 mg, 0.089 mmol) in MeCN (0.5 mL) HATU (19 mg, 0.049 mmol) was added and the mixture was stirred 1 h at rt. The mixture was diluted with MeOH/H₂O and purified by prep HPLC (C₁₈, H₂O/MeCN/0.1% TFA) to afford the title compound (15.1 mg, 58%). ESI-MS m/z calcd for [C₂₀H₂₃BrClN₇O₅S] [M+H]⁺: 588.0; found: 587.8. ¹H NMR (400 MHz, Methanol-d₄) δ 8.53 (s, 1H), 8.51 (s, 1H), 8.36 (s, 1H), 8.26 (d, J=2.4 Hz, 1H), 6.52 (d, J=2.4 Hz, 1H), 6.39 (d, J=5.4 Hz, 1H), 5.11 (dd, J=11.4, 2.7 Hz, 1H), 4.67 (dd, J=11.4, 5.4 Hz, 1H), 4.37 (t, J=6.0 Hz, 1H), 4.19 (d, J=2.3 Hz, 1H), 3.71 (dd, J=11.5, 5.4 Hz, 1H), 3.66 (dd, J=11.4, 6.9 Hz, 1H), 3.45-3.37 (m, 5H), 1.23 (t, J=7.2 Hz, 3H).

### Example 36

### 5-Bromo-2-(N-butylcarbamoyl)pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside

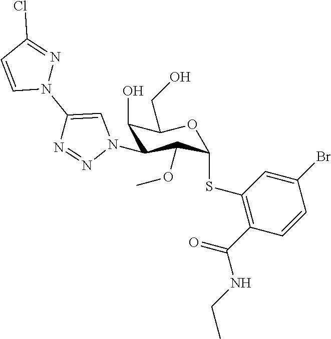

To a solution of 5-bromo-2-carboxypyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside (25 mg, 0.045 mmol), 4-methylmorpholine (20 μL, 0.18 mmol), methanesulfonic acid (8.7 μL, 0.13 mmol) and butylamine (6.5 mg, 0.089 mmol) in MeCN (0.5 mL) HATU (19 mg, 0.049 mmol) was added and the mixture was stirred 1 h at rt. The mixture was diluted with MeOH/H₂O and purified by prep HPLC (C₁₈, H₂O/MeCN/0.1% TFA) to afford the title compound (10.4 mg, 38%). ESI-MS m/z calcd for [C₂₂H₂₇BrClN₇O₅S][M+H]⁺: 616.1; found: 615.8. ¹H NMR (400 MHz, Methanol-d₄) δ 8.53 (d, J=1.7 Hz, 1H), 8.51 (d, J=1.7 Hz, 1H), 8.36 (s, 1H), 8.26 (d, J=2.5 Hz, 1H), 6.52 (d, J=2.5 Hz, 1H), 6.39 (d, J=5.4 Hz, 1H), 5.10 (dd, J=11.4, 2.7 Hz, 1H), 4.66 (dd, J=11.4, 5.5 Hz, 1H), 4.37 (t, J=5.9 Hz, 1H), 4.19 (d, J=2.1 Hz, 1H), 3.71 (dd, J=11.4, 5.3 Hz, 1H), 3.66 (dd, J=11.5, 6.7 Hz, 1H), 3.41-3.36 (m, 5H), 1.67-1.55 (m, 2H), 1.49-1.38 (m, 2H), 0.98 (t, J=7.3 Hz, 3H).

### Example 37

### 5-Bromo-2-(N-isobutylcarbamoyl)pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside

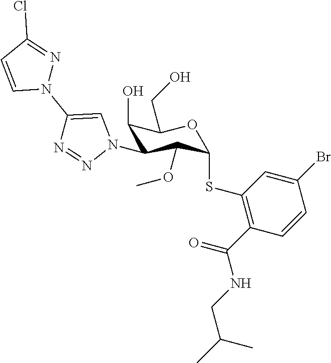

To a solution of 5-bromo-2-carboxypyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside (25 mg, 0.045 mmol), 4-methylmorpholine (20 μL, 0.18 mmol), methanesulfonic acid (8.7 μL, 0.13 mmol) and isobutylamine (6.5 mg, 0.089 mmol) in MeCN (0.5 mL) HATU (19 mg, 0.049 mmol) was added and the mixture was stirred 1 h at rt. The mixture was diluted with MeOH/H₂O and purified by prep HPLC (C₁₈, H₂O/MeCN/0.1% TFA) to afford the title compound (6.1 mg, 22%). ESI-MS m/z calcd for [C₂₂H₂₇BrClN₇O₅S][M+H]⁺: 616.1; found: 616.0. ¹H NMR (500 MHz, Methanol-d₄) δ 8.53 (d, J=1.9 Hz, 1H), 8.52 (d, J=1.9 Hz, 1H), 8.35 (s, 1H), 8.25 (d, J=2.6 Hz, 1H), 6.51 (d, J=2.5 Hz, 1H), 6.38 (d, J=5.4 Hz, 1H), 5.10 (dd, J=11.4, 2.9 Hz, 1H), 4.66 (dd, J=11.4, 5.5 Hz, 1H), 4.38 (t, J=6.1 Hz, 1H), 4.19 (d, J=2.5 Hz, 1H), 3.75-3.62 (m, 2H), 3.38 (s, 3H), 3.21 (s, 2H), 1.92 (dp, J=13.5, 6.8 Hz, 1H), 0.99 (d, J=6.7 Hz, 6H).

### Example 38

### 5-Bromo-2-[N-(2-fluoroethyl)carbamoyl]pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside

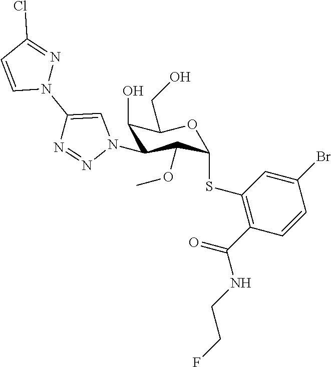

To a solution of 5-bromo-2-carboxypyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside (25 mg, 0.045 mmol), 4-methylmorpholine (20 μL, 0.18 mmol), methanesulfonic acid (8.7 μL, 0.13 mmol) and 2-fluoroethanamine hydrochloride (8.9 mg, 0.089 mmol) in MeCN (0.5 mL) HATU (19 mg, 0.049 mmol) was added and the mixture was stirred 1 h at rt. The mixture was diluted with MeOH/H₂O and purified by prep HPLC (C₁₈, H₂O/MeCN/0.1% TFA) to afford the title compound (16.4 mg, 61%). ESI-MS m/z calcd for [C₂₀H₂₂BrClFN₇O₅S] [M+H]⁺: 606.0; found: 606.0. ¹H NMR (400 MHz, Methanol-d₄) δ 8.55 (d, J=1.6 Hz, 1H), 8.52 (d, J=1.6 Hz, 1H), 8.36 (s, 1H), 8.26 (d, J=2.5 Hz, 1H), 6.52 (d, J=2.5 Hz, 1H), 6.41 (d, J=5.4 Hz, 1H), 5.11 (dd, J=11.4, 2.7 Hz, 1H), 4.71-4.60 (m, 2H), 4.51 (t, J=5.0 Hz, 1H), 4.37 (t, J=5.8 Hz, 1H), 4.18 (d, J=2.2 Hz, 1H), 3.75-3.61 (m, 4H), 3.39 (s, 3H).

### Example 39

### 5-Bromo-2-(N-bicyclo[1.1.1]pentan-1-ylcarbamoyl)pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside

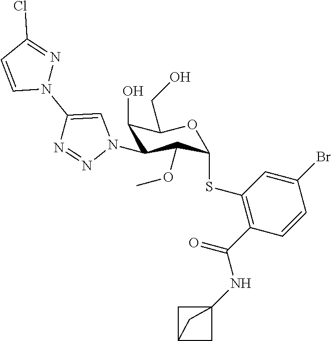

To a solution of 5-bromo-2-carboxypyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside (25 mg, 0.045 mmol), 4-methylmorpholine (20 μL, 0.18 mmol), methanesulfonic acid (8.7 μL, 0.13 mmol) and bicyclo[1.1.1]pentan-1-amine hydrochloride (10.6 mg, 0.089 mmol) in MeCN (0.5 mL) HATU (19 mg, 0.049 mmol) was added and the mixture was stirred 1 h at rt. The mixture was diluted with MeOH/H₂O and purified by prep HPLC (C₁₈, H₂O/MeCN/0.1% TFA) to afford the title compound (15.9 mg, 57%). ESI-MS m/z calcd for [C₂₃H₂₅BrClN₇O₅S] [M+H]⁺: 626.1; found: 626.1. ¹H NMR (400 MHz, Methanol-d₄) δ 8.52 (d, J=1.9 Hz, 1H), 8.49 (d, J=2.0 Hz, 1H), 8.36 (s, 1H), 8.26 (d, J=2.6 Hz, 1H), 6.52 (d, J=2.6 Hz, 1H), 6.39 (d, J=5.4 Hz, 1H), 5.12 (dd, J=11.4, 2.9 Hz, 1H), 4.67 (dd, J=11.3, 5.4 Hz, 1H), 4.37 (t, J=5.9 Hz, 1H), 4.19 (d, J=2.1 Hz, 1H), 3.71 (dd, J=11.4, 5.3 Hz, 1H), 3.66 (dd, J=11.5, 6.9 Hz, 1H), 3.39 (s, 3H), 2.48 (s, 1H), 2.20 (s, 6H).

### Example 40

### 5-Bromo-2-(N-cyclobutylcarbamoyl)pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside

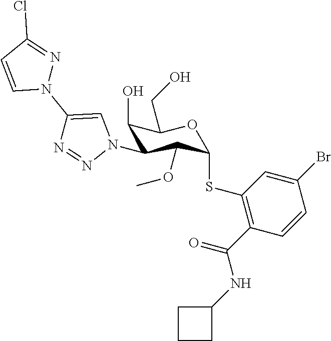

To a solution of 5-bromo-2-carboxypyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside (25 mg, 0.045 mmol), 4-methylmorpholine (20 μL, 0.18 mmol), methanesulfonic acid (8.7 μL, 0.13 mmol) and cyclobutylamine (6.3 mg, 0.089 mmol) in MeCN (0.5 mL) HATU (19 mg, 0.049 mmol) was added and the mixture was stirred 1 h at rt. The mixture was diluted with MeOH/H₂O and purified by prep HPLC (C₁₈, H₂O/MeCN/0.1% TFA) to afford the title compound (13.2 mg, 48%). ESI-MS m/z calcd for [C₂₂H₂₅BrClN₇O₅S][M+H]⁺: 614.1; found: 613.9. ¹H NMR (400 MHz, Methanol-d₄) δ 8.52 (s, 2H), 8.36 (s, 1H), 8.25 (d, J=2.4 Hz, 1H), 6.52 (d, J=2.3 Hz, 1H), 6.37 (d, J=5.4 Hz, 1H), 5.09 (dd, J=11.5, 2.6 Hz, 1H), 4.69-4.63 (m, 1H), 4.53-4.45 (m, 1H), 4.37 (t, J=5.7 Hz, 1H), 4.21-4.16 (m, 1H), 3.77-3.61 (m, 2H), 3.38 (s, 3H), 2.44-2.30 (m, 2H), 2.20-2.07 (m, 2H), 1.88-1.73 (m, 2H).

### Example 41

### 5-Bromo-2-(N-cyclopropylcarbamoyl)pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside

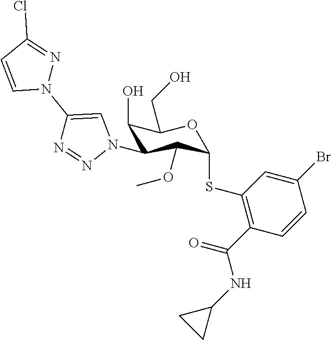

To a solution of 5-bromo-2-carboxypyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside (25 mg, 0.045 mmol), 4-methylmorpholine (20 μL, 0.18 mmol), methanesulfonic acid (8.7 μL, 0.13 mmol) and cyclopropylamine (5.1 mg, 0.089 mmol) in MeCN (0.5 mL) HATU (19 mg, 0.049 mmol) was added and the mixture was stirred 1 h at rt. The mixture was diluted with MeOH/H₂O and purified by prep HPLC (C₁₈, H₂O/MeCN/0.1% TFA) to afford the title compound (13.6 mg, 51%). ESI-MS m/z calcd for [C₂₁H₂₃BrClN₇O₅S][M+H]⁺: 600.0; found: 599.8. ¹H NMR (400 MHz, Methanol-d₄) δ 8.53 (s, 1H), 8.50 (s, 1H), 8.36 (s, 1H), 8.26 (d, J=2.5 Hz, 1H), 6.52 (d, J=2.5 Hz, 1H), 6.37 (d, J=5.4 Hz, 1H), 5.10 (dd, J=11.4, 2.8 Hz, 1H), 4.66 (dd, J=11.4, 5.4 Hz, 1H), 4.38 (t, J=5.9 Hz, 1H), 4.19 (d, J=2.3 Hz, 1H), 3.71 (dd, J=11.5, 5.3 Hz, 1H), 3.66 (dd, J=11.5, 6.9 Hz, 1H), 3.39 (s, 3H), 2.91-2.81 (m, 1H), 0.88-0.79 (m, 2H), 0.71-0.61 (m, 2H).

### Example 42

### 5-Bromo-2-cyanopyridin-3-yl 3-[4-(3,4-dichloro-1H-1,2-pyrazol-1-yl)-1H-1,2,3-triazol-1-yl]-3-deoxy-2-O-methyl-1-thio-α-D-galactopyranoside

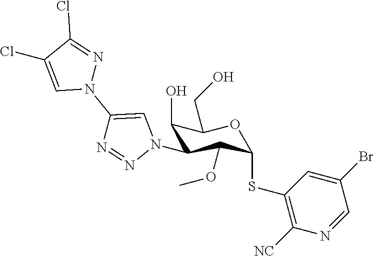

To a solution of 5-bromo-2-cyanopyridin-3-yl 3-azido-3-deoxy-2-O-methyl-1-thio-α-D-galactopyranoside (60 mg, 0.14 mmol), CuI (6 mg, 0.03 mmol) and 3,4-dichloro-1-ethynylpyrazole (28 mg, 0.17 mmol) in MeCN (2 mL) triethylamine (81.2 μL, 0.58 mmol) was added and the mixture was stirred 1 h at 40° C. More 3,4-dichloro-1-ethynylpyrazole (28 mg, 0.17 mmol) was added and the mixture was stirred an additional 3 h at 40° C. The mixture was cooled to rt, concentrated, and purified by chromatography (SiO₂, PE/EtOAc) to afford the title compound (49.9 mg, 60%). ESI-MS m/z calcd for [C₁₈H₁₆BrCl₂N₇O₄S] [M+H]⁺: 576.0; found: 575.8. ¹H NMR (400 MHz, Methanol-d₄) δ 8.71 (d, J=2.0 Hz, 1H), 8.62 (d, J=2.0 Hz, 1H), 8.45 (s, 1H), 8.42 (s, 1H), 6.50 (d, J=5.3 Hz, 1H), 5.08 (dd, J=11.3, 2.9 Hz, 1H), 4.70 (dd, J=11.3, 5.3 Hz, 1H), 4.40 (t, J=6.0 Hz, 1H), 4.20 (d, J=2.5 Hz, 1H), 3.68 (d, J=6.0 Hz, 2H), 3.46 (s, 3H)

### Example 43

### 5-Bromo-2-cyanopyridin-3-yl 3-[4-(3-chloro-4-fluoro-1H-1,2-pyrazol-1-yl)-1H-1,2,3-triazol-1-yl]-3-deoxy-2-O-methyl-1-thio-α-D-galactopyranoside

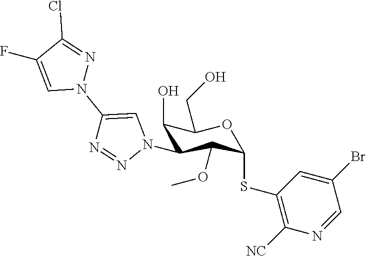

To a solution of 3-chloro-4-fluoro-1-(2,2-dichlorovinyl)pyrazole (48.5 mg, 0.23 mmol) in THF (1 mL) n-butyllithium (198 μL, 2.5 M in THF, 0.50 mmol) was added at −78° C. The mixture was stirred 10 min and was then heated to −30° C. and stirred 1 h. Acetic acid (26 μL, 0.45 mmol) was added and the mixture was heated to rt. 5-Bromo-2-cyanopyridin-3-yl 3-azido-3-deoxy-2-O-methyl-1-thio-α-D-galactopyranoside (62 mg, 0.15 mmol), CuI (6 mg, 0.03 mmol) and triethylamine (105 μL, 0.75 mmol) were added and the mixture was stirred 1 h at 50° C. The mixture was cooled to rt, filtered through a plug of celite, concentrated and purified by prep HPLC (C₁₈, H₂O/MeCN/0.1% TFA) to afford the title compound (19.2 mg, 23%). ESI-MS m/z calcd for [C₁₈H₁₆BrClFN₇O₄S] [M+H]⁺: 560.0; found: 559.8. ¹H NMR (400 MHz, Methanol-d₄) δ 8.71 (d, J=2.0 Hz, 1H), 8.62 (d, J=2.0 Hz, 1H), 8.40 (s, 1H), 8.36 (d, J=4.8 Hz, 1H), 6.50 (d, J=5.4 Hz, 1H), 5.07 (dd, J=11.3, 2.9 Hz, 1H), 4.70 (dd, J=11.3, 5.3 Hz, 1H), 4.40 (t, J=5.8 Hz, 1H), 4.20 (d, J=2.6 Hz, 1H), 3.68 (d, J=6.0 Hz, 2H), 3.46 (s, 3H).

### Intermediate 1

### 2-(3-Chloropyrazol-1-yl)ethynyl(triisopropyl)silane

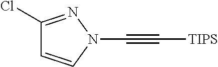

A solution of 3-chloro-1H-pyrazole (150 mg, 1.46 mmol), CuI (14 mg, 0.073 mmol), cesium carbonate (572 mg, 1.76 mmol) and 2-bromoethynyl(triisopropyl)silane (765 mg, 2.93 mmol) in 1,4-dioxane (2 mL) and PEG400 (400 mg) was stirred 4 h at 70° C. The mixture was filtered through a plug of celite, concentrated and purified by chromatography (SiO₂, PE/EtOAc) to afford the product (68 mg, 16%). ESI-MS m/z calcd for [C₁₄H₂₃ClN₂Si] [M+H]⁺: 283.1; found: 283.1.

### 4-Methylphenyl 3-azido-4,6-O-benzylidene-3-deoxy-1-thio-β-D-galactopyranoside

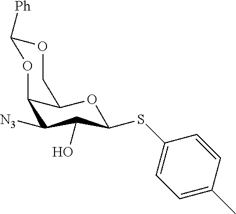

To a solution of 1,2,4,6-tetra-O-acetyl-3-azido-3-deoxy-β-D-galactopyranose (30.0 g, 78.7 mmol) and 4-methylbenzenethiol (11.0 g, 86.6 mmol) in DCM (200 mL) boron trifluoride diethyl etherate (30.2 mL, 236 mmol) was added and the mixture was stirred 1 h at rt. The mixture was partitioned between cold water and DCM. Aqueous NaOH (5 M, 140 mL) was added to maintain pH at approximately at 7. The organic phase was dried, concentrated and the residue was triturated from PE. The obtained material was stirred 19 h at rt in MeOH (300 mL) and NaOMe (1 M, 13 mL). The mixture was neutralized with silica (30 g) and filtered. The filtrate was evaporated, and the residue was dissolved in MeCN (300 mL). To the solution benzaldehyde dimethylacetal (17.9 mL, 118 mmol) followed by p-toluenesulfonic acid monohydrate (1.0 g, 5.26 mmol) were added and the mixture was stirred 1 h at rt. The mixture was neutralized with ammonia (16 M, 1.0 mL) and water (200 mL) was added. The precipitate was isolated as the product (26.61 g, 85%). ESI-MS m/z calcd for [C₂₀H₂₁N₃O₄S] [M+Na]⁺: 422.1; found: 422.1. ¹H NMR (400 MHz, Methanol-d₄) δ 7.56-7.51 (m, 2H), 7.42 (m, 2H), 7.35 (m, 3H), 7.05 (d, J=7.9 Hz, 2H), 5.59 (s, 1H), 4.57 (d, J=9.4 Hz, 1H), 4.32-4.27 (m, 1H), 4.21 (dd, J=12.4, 1.6 Hz, 1H), 4.09 (dd, J=12.4, 1.6 Hz, 1H), 3.81 (t, J=9.7 Hz, 1H), 3.63-3.58 (m, 1H), 3.44 (dd, J=10.0, 3.3 Hz, 1H), 2.31 (s, 3H).

### 4-Methylphenyl 4,6-di-O-acetyl-3-azido-3-deoxy-2-O-methyl-1-thio-β-D-galactopyranoside

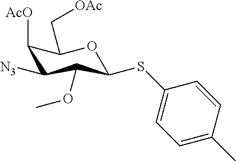

To a cooled (0° C.) solution of 4-methylphenyl 3-azido-4,6-O-benzylidene-3-deoxy-1-thio-β-D-galactopyranoside (26.61 g, 66.6 mmol) and 4-methylbenzenethiol (11.0 g, 86.6 mmol) in DMF (220 mL) NaH (60% in oil, 5.32 g, 133 mmol) was added and the mixture was stirred 5 min. A solution of iodomethane (6.33 mL, 100 mmol) in DMF (50 mL) was added over 15 min and the resulting mixture was stirred 30 min at rt. The reaction was quenched by addition of MeOH (5.0 mL) and ice/water (200 mL) was added. The precipitate was collected, washed with water, dried and stirred 2 h at rt in TFA/water (170 mL, 4:1). The mixture was cooled in an ice-bath and ammonia (16 M, 120 mL) was added cautiously. The precipitate was isolated, dissolved in pyridine (50 mL) and evaporated. The residue was stirred 4 h at 40° C. in pyridine (120 mL) and acetic anhydride (75 mL). The mixture was concentrated and partitioned between EtOAc and HCl (1 M). The organic phase was dried, evaporated and purified by chromatography (SiO₂, PE/EtOAc) to afford the product (26.72 g, 94%). ESI-MS m/z calcd for [C₁₈H₂₃N₃O₆S] [M+NH₄]⁺: 427.1; found: 427.2. ¹H NMR (400 MHz, Chloroform-d) δ 7.48 (d, J=8.1 Hz, 2H) 7.13 (d, J=8.0 Hz, 2H), 5.35 (d, J=3.0 Hz, 1H), 4.53 (d, J=9.6 Hz, 1H), 4.10 (d, J=6.5 Hz, 2H), 3.80 (t, J=6.5 Hz, 1H), 3.68 (s, 3H), 3.57 (dd, J=9.6, 3.3 Hz, 1H), 3.38 (t, J=9.6 Hz, 1H), 2.35 (s, 3H), 2.15 (s, 3H), 2.05 (s, 3H).

### 4,6-Di-O-acetyl-3-azido-3-deoxy-2-O-methyl-α-D-galactopyranosyl trichloroacetimidate

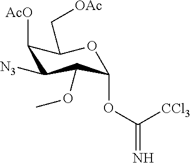

To a cooled (0° C.) solution of 4-methylphenyl 4,6-di-O-acetyl-3-azido-3-deoxy-2-O-methyl-1-thio-β-D-galactopyranoside (6.41 g, 15.6 mmol) in 1,4-dioxane (60 mL) and water (9.3 mL)N-bromosuccinimide (9.7 g, 55 mmol) was added in portions and the mixture was stirred 1 h at rt. The mixture was diluted with EtOAc and washed with aq NaHSO₃ (1 M), saturated aq NaHCO₃ and brine. The organic phase was evaporated and purified by chromatography (SiO₂, PE/EtOAc). The obtained material was dissolved in DCM (30 mL) and trichloroacetonitrile (1.40 mL, 13.4 mmol) was added followed by 1,8-diazabicyclo[5.4.0]undec-7-ene (0.15 mL, 0.96 mmol). After stirring 50 min at rt the mixture was concentrated and purified by chromatography (SiO₂, PE/EtOAc) to afford the product (3.65 g, 52%). ¹H NMR (400 MHz, Chloroform-d) δ 8.70 (s, 1H), 6.64 (d, J=3.3 Hz, 1H), 5.47 (d, J=2.7 Hz, 1H), 4.36 (t, J=6.5 Hz, 1H), 4.15 (dd, J=11.4, 6.2 Hz, 1H), 4.01 (dd, J=11.5, 4.6 Hz, 1H), 3.98 (dd, J=10.5, 3.3 Hz, 1H), 3.79 (dd, J=10.5, 3.3 Hz, 1H), 3.54 (s, 3H), 2.18 (s, 3H), 2.03 (s, 3H).

### Triisopropylsilyl 4,6-di-O-acetyl-3-azido-3-deoxy-2-O-methyl-1-thio-α-D-galactopyranoside

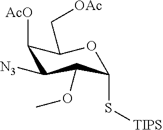

To a solution of 4,6-di-O-acetyl-3-azido-3-deoxy-2-O-methyl-α-D-galactopyranosyl trichloroacetimidate (2.00 g, 4.46 mmol) in DCM (20 mL) triisopropylsilylthiol (1.29 mL, 5.8 mmol) was added followed by boron trifluoride diethyl etherate (0.11 mL, 0.89 mmol) was added and the mixture was stirred 1 h at rt. The mixture was washed with saturated aq NaHCO₃ and the organic phase was evaporated and purified by chromatography (SiO₂, PE/EtOAc) to afford the product (1.70 g, 80%). ESI-MS m/z calcd for [C₂₀H₃₇N₃O₆SSi] [M+Na]⁺: 498.2; found: 498.2. ¹H NMR (400 MHz, Chloroform-d) δ 5.75 (d, J=5.0 Hz, 1H), 5.39 (d, J=2.5 Hz, 1H), 4.65 (t, J=6.5 Hz, 1H), 4.09 (dd, J=11.4, 6.6 Hz, 1H), 4.03-3.98 (m, 1H), 3.96 (dd, J=10.0, 2.5 Hz, 1H), 3.79 (dd, J=10.5, 5.0 Hz, 1H), 3.52 (s, 3H), 2.15 (s, 3H), 2.04 (s, 3H), 1.31 (m, 3H), 1.15 (d, J=7.3 Hz, 18H).

### 5-Bromo-2-cyanophenyl 4,6-di-O-acetyl-3-azido-3-deoxy-2-O-methyl-1-thio-α-D-galactopyranoside

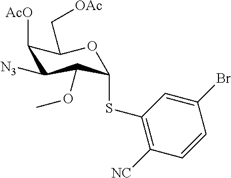

To a solution of triisopropylsilyl 4,6-di-O-acetyl-3-azido-3-deoxy-2-O-methyl-1-thio-α-D-galactopyranoside (970 mg, 2.04 mmol) and 4-bromo-2-fluorobenzonitrile (489 mg, 2.45 mmol) in MeCN (30 mL) TBAF (0.20 mL, 1 M in THF, 0.20 mmol) was added and the mixture was stirred 30 min at rt. The mixture was concentrated and partitioned between EtOAc and HCl (1 M). The organic phase was dried, concentrated and purified by chromatography (SiO₂, PE/EtOAc) to afford the product (822 mg, 81%). ESI-MS m/z calcd for [C₁₈H₁₉BrN₄O₆S] [M+Na]⁺: 521.0; found: 521.0. ¹H NMR (500 MHz, Chloroform-d) δ 7.86 (s, 1H), 7.54 (d, J=0.9 Hz, 2H), 6.11 (d, J=5.3 Hz, 1H), 5.41 (d, J=2.6 Hz, 1H), 4.57-4.51 (m, 1H), 4.05 (dd, J=11.6, 5.1 Hz, 1H), 4.02-3.95 (m, 2H), 3.86 (dd, J=10.4, 3.3 Hz, 1H), 3.61 (s, 3H), 2.15 (s, 3H), 1.96 (s, 3H).

### 5-Bromo-2-(N-methyl-carbonyl)phenyl 3-azido-3-deoxy-2-O-methyl-1-thio-α-D-galactopyranoside

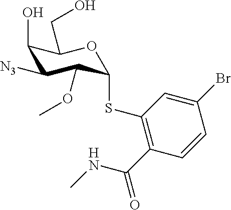

A solution of 5-bromo-2-cyanophenyl 4,6-di-O-acetyl-3-azido-3-deoxy-2-O-methyl-1-thio-α-D-galactopyranoside (793 mg, 1.59 mmol) in EtOH (16 mL) and NaOH (3 M, 8 mL) was stirred 24 h at 80° C. The mixture was concentrated to approximately half its volume. The mixture was acidified to pH 1 by addition of HCl (5 M). The precipitate was isolated by filtration to afford the intermediate carboxylic acid (259 mg). The filtrate was extracted with EtOAc, dried and evaporated to afford more of the intermediate carboxylic acid (468 mg). The carboxylic acid (727 mg) was dissolved together with 1-hydroxybenzotriazole hydrate (292 mg, 1.91 mmol) and N-(3-dimethylaminopropyl)-N′-ethylcarbodiimide hydrochloride (366 mg, 1.91 mmol) in DMF (8 mL). Methylamine (0.70 mL, 8 M in EtOH, 5.57 mmol) was added and the mixture was stirred 7 h at 50° C., then 15 h at rt. The mixture was diluted with EtOAc, washed with water and the aqueous phase was extracted with EtOAc. The combined organic phases were dried, evaporated and purified by chromatography (SiO₂, PE/EtOAc) to afford the product (455 mg, 64%). ESI-MS m/z calcd for [C₁₅H₁₉BrN₄O₅S] [M+Na]⁺: 469.0; found: 469.0. ¹H NMR (500 MHz, Methanol-d₄) δ 7.94 (d, J=1.9 Hz, 1H), 7.52 (dd, J=8.2, 1.9 Hz, 1H), 7.29 (d, J=8.2 Hz, 1H), 5.97 (d, J=5.4 Hz, 1H), 4.25 (t, J=6.4 Hz, 1H), 4.02 (dd, J=10.7, 5.4 Hz, 1H), 3.99 (d, J=2.0 Hz, 1H), 3.68 (dd, J=11.4, 5.5 Hz, 1H), 3.63 (dd, J=11.4, 6.8 Hz, 1H), 3.58 (dd, J=10.7, 3.0 Hz, 1H), 3.50 (s, 3H), 2.90 (s, 3H).

### Intermediate 3

### 5-Bromo-2-cyanopyridin-3-yl 4,6-di-O-acetyl-3-azido-3-deoxy-2-O-methyl-1-thio-α-D-galactopyranoside

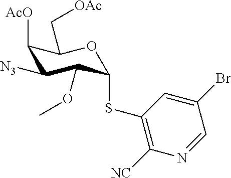

To a solution of triisopropylsilyl 4,6-di-O-acetyl-3-azido-3-deoxy-2-O-methyl-1-thio-α-D-galactopyranoside (400 mg, 0.84 mmol) and 5-bromo-3-fluoropyridine-2-carbonitrile (210 mg, 1.01 mmol) in MeCN (4.0 mL) TBAF (84 μL, 1 M in THF, 0.084 mmol) was added and the mixture was stirred 5 min at rt. The mixture was partitioned between EtOAc, brine and HCl (1 mL, 1 M). The organic phase was dried, evaporated and purified by chromatography (SiO₂, PE/EtOAc) to afford the product (394 mg, 94%). ESI-MS m/z calcd for [C₁₇H₁₈BrN₅O₆S] [M+H]⁺: 500.0; found: 500.0. ¹H NMR (400 MHz, Chloroform-d) δ 8.65 (d, J=2.0 Hz, 1H), 8.21 (d, J=2.0 Hz, 1H), 6.12 (d, J=5.3 Hz, 1H), 5.43 (d, J=2.7 Hz, 1H), 4.53-4.46 (m, 1H), 4.06 (dd, J=11.7, 4.7 Hz, 1H), 4.01 (dd, J=10.3, 5.3 Hz, 1H), 3.98 (dd, J=11.7, 7.6 Hz, 1H), 3.87 (dd, J=10.3, 3.3 Hz, 1H), 3.62 (s, 3H), 2.16 (s, 3H), 1.98 (s, 3H).

### Intermediate 4

### 2-(3-Fluoropyrazol-1-yl)ethynyl(triisopropyl)silane

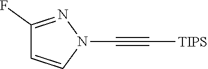

A solution of 3-fluoro-1H-pyrazole (75 mg, 0.87 mmol), CuI (8.3 mg, 0.044 mmol), cesium carbonate (341 mg, 1.05 mmol) and 2-bromoethynyl(triisopropyl)silane (455 mg, 1.74 mmol) in 1,4-dioxane (1 mL) and PEG400 (200 mg) was stirred 2 h at 70° C. The mixture was filtered through a plug of celite, concentrated and purified by chromatography (SiO₂, PE/EtOAc) to afford the product (40 mg, 17%). ESI-MS m/z calcd for [C₁₄H₂₃FN₂Si] [M+H]⁺: 267.2; found: 267.2.

### 2,4,6-Tri-O-acetyl-3-azido-3-deoxy-β-D-galactopyranosyl chloride

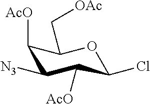

A solution of 1,2,4,6-tetra-O-acetyl-3-azido-3-deoxy-β-D-galactopyranoside (12.0 g, 32.1 mmol), PCl₅ (7.5 g, 36.0 mmol) and boron trifluoride diethyl etherate (50 μL, 0.41 mmol) in DCM (150 mL) was stirred 1 h at rt. The mixture was partitioned between saturated aq NaHCO₃ and DCM. The organic phase was dried, concentrated, and the residue was triturated in diethyl ether/PE to afford the product as a crystalline solid (10.2 g, 91%). ¹H NMR (400 MHz, Chloroform-d) δ 5.48 (d, J=3.2 Hz, 1H), 5.34 (t, J=9.2 Hz, 1H), 5.24 (d, J=8.7 Hz, 1H), 4.18 (dd, J=11.5, 6.1 Hz, 1H), 4.10 (dd, J=11.6, 6.7 Hz, 1H), 3.98 (t, J=6.4 Hz, 1H), 3.60 (dd, J=10.3, 3.3 Hz, 1H), 2.20 (s, 3H), 2.17 (s, 3H), 2.07 (s, 3H).

### 5-Chloropyridin-3-yl 2,4,6-tri-O-acetyl-3-azido-3-deoxy-1-thio-α-D-galactopyranoside

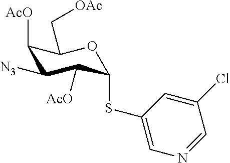

To a solution of 2,4,6-tri-O-acetyl-3-azido-3-deoxy-β-D-galactopyranosyl chloride (1.72 g, 4.91 mmol) and 5-chloropyridine-3-thiol (650 mg, 4.46 mmol) in DMF (20 mL) NaH (60% in oil, 428 mg, 11.2 mmol) was added and the mixture was stirred 3 h at rt. The mixture was diluted with EtOAc and washed twice with water and once with brine. The organic phase was dried, concentrated and purified by chromatography (SiO₂, PE/EtOAc) to afford the product (1.17 g, 57%). ESI-MS m/z calcd for [C₁₇H₁₉ClN₄O₇S] [M+H]⁺: 459.1; found: 459.1. ¹H NMR (400 MHz, Chloroform-d) δ 8.53 (d, J=1.8 Hz, 1H), 8.50 (d, J=2.2 Hz, 1H), 7.84 (t, J=2.1 Hz, 1H), 5.99 (d, J=5.5 Hz, 1H), 5.50 (d, J=3.3 Hz, 1H), 5.30 (dd, J=10.9, 5.5 Hz, 1H), 4.68-4.60 (m, 1H), 4.14 (dd, J=11.7, 4.6 Hz, 2H), 4.03 (dd, J=11.6, 7.9 Hz, 1H), 3.96 (dd, J=10.9, 3.4 Hz, 1H), 2.21 (s, 3H), 2.18 (d, J=2.1 Hz, 3H), 2.04 (s, 3H).

### Intermediate 6

### 5-Chloro-2-(trifluoromethyl)pyridin-3-yl 4,6-di-O-acetyl-3-azido-3-deoxy-2-O-methyl-1-thio-α-D-galactopyranoside

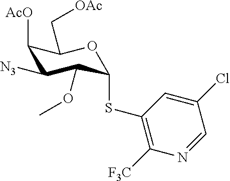

To a solution of triisopropylsilyl 4,6-di-O-acetyl-3-azido-3-deoxy-2-O-methyl-1-thio-α-D-galactopyranoside (400 mg, 0.84 mmol) and 5-chloro-3-fluoro-2-(trifluoromethyl)pyridine (201 mg, 1.01 mmol) in MeCN (10 mL) TBAF (0.084 mL, 1 M in THF, 0.084 mmol) was added and the mixture was stirred overnight at rt. The mixture was concentrated and partitioned between EtOAc and HCl (1 M). The organic phase was dried, concentrated and purified by chromatography (SiO₂, PE/EtOAc) to afford the product (185 mg, 44%). ESI-MS m/z calcd for [C₁₇H₁₈ClF₃N₄O₆S] [M+H]⁺: 499.1; found: 499.0. ¹H NMR (400 MHz, Chloroform-d) δ 8.51 (s, 1H), 8.15 (s, 1H), 5.98 (d, J=5.4 Hz, 1H), 5.43 (s, 1H), 4.56-4.48 (m, 1H), 4.13-3.98 (m, 3H), 3.85 (dd, J=10.3, 3.3 Hz, 1H), 3.56 (s, 3H), 2.18 (s, 3H), 2.00 (s, 3H).

### Intermediate 7

### 5-Bromo-2-(trifluoromethyl)pyridin-3-yl 4,6-di-O-acetyl-3-azido-3-deoxy-2-O-methyl-1-thio-α-D-galactopyranoside AcO

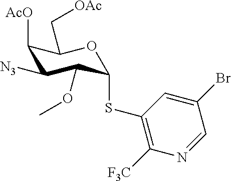

To a solution of triisopropylsilyl 4,6-di-O-acetyl-3-azido-3-deoxy-2-O-methyl-1-thio-α-D-galactopyranoside (400 mg, 0.84 mmol) and 5-bromo-3-fluoro-2-(trifluoromethyl)pyridine (246 mg, 1.01 mmol) in MeCN (10 mL) TBAF (0.084 mL, 1 M in THF, 0.084 mmol) was added and the mixture was stirred overnight at rt. The mixture was concentrated and partitioned between EtOAc and HCl (1 M). The organic phase was dried, concentrated and purified by chromatography (SiO₂, PE/EtOAc) to afford the product (148 mg, 32%). ESI-MS m/z calcd for [C₁₇H₁₈BrF₃N₄O₆S] [M+H]⁺: 543.0; found: 543.0. ¹H NMR (400 MHz, Chloroform-d) δ 8.61 (s, 1H), 8.29 (s, 1H), 5.99 (d, J=5.2 Hz, 1H), 5.44-5.41 (m, 1H), 4.54-4.48 (m, 1H), 4.11-3.97 (m, 3H), 3.87 (s, 1H), 3.56 (s, 3H), 2.18 (s, 3H), 2.00 (s, 3H).

### Intermediate 8

### 3-Chloro-2-(trifluoromethyl)pyridin-5-yl 4,6-di-O-acetyl-3-azido-3-deoxy-2-O-methyl-1-thio-α-D-galactopyranoside

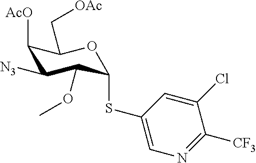

To a solution of 2-bromo-3-chloro-5-fluoropyridine (600 mg, 2.85 mmol) in DMF (3 mL) CuI (1.09 g, 5.70 mmol) and methyl 2,2-difluoro-2-fluorosulfonyl-acetate (0.73 mL, 5.70 mmol) were added and the mixture was stirred 7 h at 110° C. The mixture was diluted with water (100 mL) and extracted with diethyl ether. The combined organic phases were washed with brine, dried, and concentrated to afford a clear yellow oil (530 mg). To a solution of the clear yellow oil (300 mg) and triisopropylsilyl 4,6-di-O-acetyl-3-azido-3-deoxy-2-O-methyl-1-thio-α-D-galactopyranoside (600 mg, 1.26 mmol) in MeCN (19 mL) TBAF (0.31 mL, 1 M in THF, 0.31 mmol) was added and the mixture was stirred 1 h at rt. The mixture was concentrated and partitioned between EtOAc and HCl (1 M). The organic phase was dried, concentrated and purified by chromatography (SiO₂, PE/EtOAc) to afford the product (241 mg, 38%). ESI-MS m/z calcd for [C₁₇H₁₈ClF₃N₄O₆S] [M+H]⁺: 499.1; found: 499.0. ¹H NMR (400 MHz, Chloroform-d) δ 8.61 (d, J=1.8 Hz, 1H), 8.01 (d, J=1.4 Hz, 1H), 6.06 (d, J=5.3 Hz, 1H), 5.42 (d, J=2.4 Hz, 1H), 4.50 (dd, J=7.7, 4.1 Hz, 1H), 4.16-3.97 (m, 4H), 3.82 (dd, J=10.4, 3.3 Hz, 1H), 3.58 (s, 3H), 2.17 (s, 4H), 1.96 (s, 3H).

### Intermediate 9

### 3-Bromo-2-(trifluoromethyl)pyridin-5-yl 4,6-di-O-acetyl-3-azido-3-deoxy-2-O-methyl-1-thio-α-D-galactopyranoside

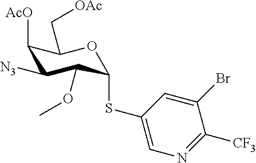

To a solution of triisopropylsilyl 4,6-di-O-acetyl-3-azido-3-deoxy-O-methyl-1-thio-α-D-galactopyranoside (600 mg, 1.26 mmol) and 3-bromo-5-fluoro-2-(trifluoromethyl)pyridine (400 mg, 1.64 mmol) in MeCN (10 mL) TBAF (0.13 mL, 1 M in THF, 0.13 mmol) was added and the mixture was stirred 2 days at rt. The mixture was concentrated and partitioned between EtOAc and HCl (1 M). The organic phase was dried, concentrated and purified by chromatography (SiO₂, PE/EtOAc) to afford the product (373 mg, 54%). ESI-MS m/z calcd for [C₁₇H₁₈BrF₃N₄O₆S] [M+H]⁺: 543.0; found: 543.0. ¹H NMR (400 MHz, Chloroform-d) δ 8.65 (s, 1H), 8.19 (s, 1H), 6.07 (d, J=5.2 Hz, 1H), 5.42 (d, J=3.2 Hz, 1H), 4.50 (dd, J=7.6, 3.9 Hz, 1H), 4.15-3.94 (m, 3H), 3.82 (dd, J=10.3, 3.2 Hz, 1H), 3.58 (s, 3H), 2.18 (s, 3H), 1.97 (s, 3H).

### Intermediate 10

### 5-Bromo-2-cyanopyridin-3-yl 4,6-di-O-acetyl-3-[4-(3-chloro-1H-1,2-pyrazol-1-yl)-1H-1,2,3-triazol-1-yl]-3-deoxy-2-O-methyl-1-thio-α-D-galactopyranoside

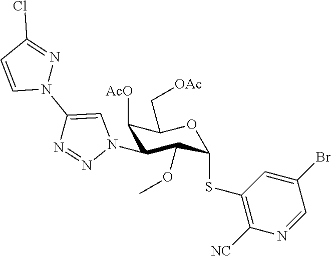

To a solution of 5-bromo-2-cyanopyridin-3-yl 4,6-di-O-acetyl-3-azido-3-deoxy-2-O-methyl-1-thio-α-D-galactopyranoside (920 mg, 1.84 mmol), CuI (88 mg, 0.46 mmol) and 2-(3-chloropyrazol-1-yl)ethynyl(triisopropyl)silane (676 mg, 2.39 mmol) in MeCN (10 mL) DIPEA (0.94 mL, 5.52 mmol) and TBAF (0.045 mL, 1M, in THF, 0.045 mmol) were added and the mixture was stirred overnight at 60° C. Additional TBAF (1.84 mL, 1M, 1.84 mmol) was added and the mixture was stirred overnight at 60° C. The mixture was concentrated and purified by chromatography (SiO₂, PE/EtOAc) to afford the product (564 mg, 48%). ESI-MS m/z calcd for [C₂₂H₂₁BrClN₇O₆S] [M+H]⁺: 626.0; found: 625.9. ¹H NMR (400 MHz, Chloroform-d) 8.68 (d, J=2.0 Hz, 1H), 8.25 (d, J=2.0 Hz, 1H), 8.20 (d, J=2.5 Hz, 1H), 7.90 (s, 1H), 6.37 (d, J=2.5 Hz, 1H), 6.32 (d, J=5.3 Hz, 1H), 5.61 (d, J=2.5 Hz, 1H), 5.03 (dd, J=11.1, 2.9 Hz, 1H), 4.81-4.67 (m, 2H), 4.17-3.97 (m, 2H), 3.46 (s, 3H), 2.07 (s, 3H), 1.96 (s, 3H).

### 5-Bromo-2-carboxypyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside

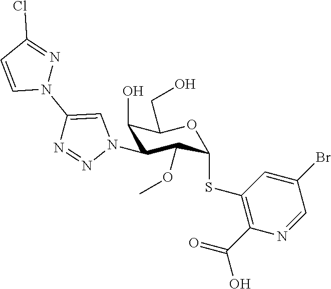

A solution of 5-bromo-2-cyanopyridin-3-yl 4,6-di-O-acetyl-3-[4-(3-chloro-1H-1,2-pyrazol-1-yl)-1H-1,2,3-triazol-1-yl]-3-deoxy-2-O-methyl-1-thio-α-D-galactopyranoside (564 mg, 0.90 mmol) in EtOH (10 mL) and NaOH (3 M, 5 mL) was stirred 72 h at 40° C. The mixture was concentrated to approximately half its volume. The mixture was acidified to pH 1 by addition of HCl (5 M) and extracted with EtOAc. The organic phase was dried and concentrated to afford the product (437 mg, 87%). ESI-MS m/z calcd for [C₁₈H₁₈BrClN₆O₆S] [M+H]⁺: 561.0; found: 560.7. ¹H NMR (400 MHz, Methanol-d₄) δ 8.58 (d, J=2.0 Hz, 1H), 8.56 (d, J=2.0 Hz, 1H), 8.37 (s, 1H), 8.26 (d, J=2.6 Hz, 1H), 6.52 (d, J=2.6 Hz, 1H), 6.41 (d, J=5.4 Hz, 1H), 5.09 (dd, J=11.4, 2.9 Hz, 1H), 4.68 (dd, J=11.2, 5.4 Hz, 1H), 4.38 (t, J=6.3 Hz, 1H), 4.20 (d, J=2.3 Hz, 1H), 3.75-3.64 (m, 2H), 3.40 (s, 3H).

### Intermediate 11

### 2-(4-Chloropyrazol-1-yl)ethynyl(triisopropyl)silane

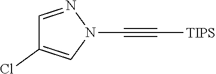

A solution of 4-chloro-1H-pyrazole (513 mg, 5.0 mmol), CuI (38 mg, 0.2 mmol), cesium carbonate (2.12 g, 6.5 mmol) and 2-bromoethynyl(triisopropyl)silane (1.70 g, 6.5 mmol) in 1,4-dioxane (6.5 mL) and PEG400 (1.44 g) was stirred 2 h at 70° C. The mixture was filtered through a plug of celite, concentrated and purified by chromatography (SiO₂, PE/EtOAc) to afford the product (75 mg, 5%). ESI-MS m/z calcd for [C₁₄H₂₃ClN₂Si] [M+H]⁺: 283.1; found: 282.9. ¹H NMR (400 MHz, Chloroform-d) δ 7.69 (s, 1H), 7.55 (s, 1H), 1.17-1.10 (m, 21H).

### 5-Bromo-2-cyanopyridin-3-yl 3-azido-3-deoxy-2-O-methyl-1-thio-α-D-galactopyranoside

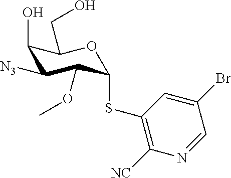

To a solution of 5-bromo-2-cyanopyridin-3-yl 4,6-di-O-acetyl-3-azido-3-deoxy-2-O-methyl-1-thio-α-D-galactopyranoside (927 mg, 1.85 mmol) in MeOH (5 mL) NaOMe (19 μL, 0.19 mmol) was added and the mixture was stirred 1 h at rt. The mixture was quenched with acetic acid (5 μL) and concentrated to afford the product (757 mg, 98%). ESI-MS m/z calcd for [C₁₃H₁₄BrN₅O₄S] [M+Na]⁺: 438.0; found: 438.0. ¹H NMR (400 MHz, Methanol-d₄) δ 8.67 (d, J=2.0 Hz, 1H), 8.56 (d, J=2.1 Hz, 1H), 6.31 (d, J=5.3 Hz, 1H), 4.17 (t, J=5.8 Hz, 1H), 4.10 (dd, J=10.5, 5.3 Hz, 1H), 4.02-3.97 (m, 1H), 3.68 (dd, J=10.6, 3.0 Hz, 1H), 3.64 (d, J=5.9 Hz, 2H), 3.57 (s, 3H).

### Intermediate 13

### 2-(4-Fluoropyrazol-1-yl)ethynyl(triisopropyl)silane

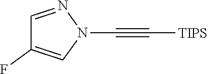

A solution of 4-fluoro-1H-pyrazole (430 mg, 5.0 mmol), CuI (38 mg, 0.2 mmol), cesium carbonate (2.12 g, 6.5 mmol) and 2-bromoethynyl(triisopropyl)silane (1.70 g, 6.5 mmol) in 1,4-dioxane (6.5 mL) and PEG400 (1.44 g) was stirred 6 h at 70° C. The mixture was filtered through a plug of celite, concentrated and purified by chromatography (SiO₂, PE/EtOAc) to afford the product (158 mg, purity 49%, 6%). ESI-MS m/z calcd for [C₁₄H₂₃FN₂Si] [M+H]⁺: 267.2; found: 267.0. H NMR (400 MHz, Chloroform-d) δ 7.57 (d, J=4.8 Hz, 1H), 7.52 (d, J=3.6 Hz, 1H), 1.18-1.09 (m, 21H).

### Intermediate 14

### 2-(3-Methylpyrazol-1-yl)ethynyl(triisopropyl)silane

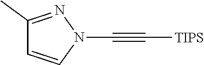

### 2-(5-Methylpyrazol-1-yl)ethynyl(triisopropyl)silane

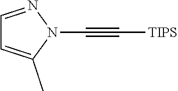

A solution of 3-methyl-1H-pyrazole (411 mg, 5.0 mmol), CuI (38 mg, 0.2 mmol), cesium carbonate (2.12 g, 6.5 mmol) and 2-bromoethynyl(triisopropyl)silane (1.70 g, 6.5 mmol) in 1,4-dioxane (6.5 mL) and PEG400 (1.44 g) was stirred 2 h at 70° C. The mixture was filtered through a plug of celite, concentrated and purified by chromatography (SiO₂, PE/EtOAc) to afford 2-(3-methylpyrazol-1-yl)ethynyl(triisopropyl)silane (42 mg, 3%) and 2-(5-methylpyrazol-1-yl)ethynyl(triisopropyl)silane (37 mg, 3%). 2-(3-Methylpyrazol-1-yl)ethynyl(triisopropyl)silane ESI-MS m/z calcd for [C₁₅H₂₆N₂Si] [M+H]⁺: 263.2; found: 262.9. ¹H NMR (400 MHz, Chloroform-d) δ 7.58 (d, J=2.5 Hz, 1H), 6.09 (d, J=2.4 Hz, 1H), 2.32 (s, 3H), 1.19-1.08 (m, 21H).

### 2-(5-Methylpyrazol-1-yl)ethynyl(triisopropyl)silane

ESI-MS m/z calcd for [C₁₅H₂₆N₂Si] [M+H]⁺: 263.2; found: 262.9. ¹H NMR (400 MHz, Chloroform-d) δ 7.49 (d, J=1.5 Hz, 1H), 6.05 (d, J=0.8 Hz, 1H), 2.40 (s, 3H), 1.23-1.08 (m, 21H).

### Intermediate 16

### 2-(3-Chloro-5-methylpyrazol-1-yl)ethynyl(triisopropyl)silane

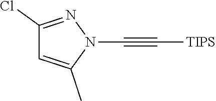

To a solution of 3-chloro-5-methyl-1H-pyrazole (291 mg, 2.5 mmol), copper(II) acetate (545 mg, 3.0 mmol) and Na₂CO₃ (132 mg, 1.25 mmol) in toluene (6.25 mL) pyridine (0.5 mL, 6.25 mmol) was added followed by ethynyl(triisopropyl)silane (0.84 mL, 3.75 mmol). The mixture was stirred overnight at 70° C. and was then filtered through a plug of celite. The filtrate was concentrated and purified by chromatography (SiO₂, PE/EtOAc) to afford the product (135 mg, 18%). ESI-MS m/z calcd for [C₁₅H₂₅ClN₂Si][M+H]⁺: 297.2; found: 296.8. ¹H NMR (400 MHz, Chloroform-d) δ 6.01 (s, 1H), 2.38 (d, J=0.7 Hz, 3H), 1.15-1.11 (m, 21H).

### Intermediate 17

### 2-(3-Chloro-5-methylpyrazol-1-yl)ethynyl(triisopropyl)silane

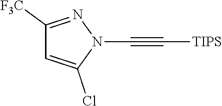

To a solution of 3-chloro-5-(trifluoromethyl)-1H-pyrazole (426 mg, 2.5 mmol), copper(II) acetate (545 mg, 3.0 mmol) and Na₂CO₃ (132 mg, 1.25 mmol) in toluene (6.25 mL) pyridine (0.5 mL, 6.25 mmol) was added followed by ethynyl(triisopropyl)silane (0.84 mL, 3.75 mmol). The mixture was stirred overnight at 70° C. and was then filtered through a plug of celite. The filtrate was concentrated and purified by chromatography (SiO₂, PE/EtOAc) to afford the product (80 mg, 9%). ESI-MS m/z calcd for [C₁₅H₂₂ClF₃N₂Si] [M+H]⁺: 351.1; found: 350.8. ¹H NMR (400 MHz, Chloroform-d) δ 6.54 (s, 1H), 1.17-1.12 (m, 21H).

### Intermediate 18

### 3-Chloro-4-methylpyrazole-1-carbaldehyde

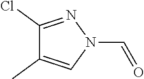

Acetic anhydride (956 μL, 10.1 mmol) was added to formic acid (3.18 mL, 84.3 mmol) and the mixture was stirred 20 min at rt. The mixture was added to 3-chloro-4-methyl-1H-pyrazole (393 mg, 3.37 mmol) and stirred 20 min at rt. The mixture was concentrated to afford the product (487 mg, 99%). ESI-MS m/z calcd for [C₅H₅ClN₂O][M+H]⁺: 145.0; found: 145.2. ¹H NMR (400 MHz, Chloroform-d) δ 8.98 (s, 1H), 7.95 (s, 1H), 2.11 (d, J=1.0 Hz, 3H).

### 3-Chloro-1-(2,2-dichlorovinyl)-4-methylpyrazole

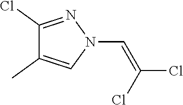

To a solution of 3-chloro-4-methylpyrazole-1-carbaldehyde (487 mg, 3.37 mmol) and triphenylphosphine (2.65 g, 10.1 mmol) in THF (26 mL) carbon tetrachloride (3.26 mL, 33.7 mmol) was added and the mixture was stirred overnight at 60° C. The mixture was cooled to rt and filtered through a celite plug. The filtrate was concentrated and purified by chromatography (SiO₂, PE/EtOAc) to afford the product (584 mg, 82%). ESI-MS m/z calcd for [C₆H₅Cl₃N₂] [M+H]⁺: 211.0; found: 211.3. ¹H NMR (400 MHz, Chloroform-d) δ 7.91 (s, 1H), 7.33 (s, 1H), 2.07 (d, J=0.8 Hz, 3H).

### 3-Chloro-1-ethynyl-4-methylpyrazole

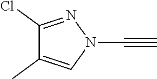

To a solution of 3-chloro-1-(2,2-dichlorovinyl)-4-methylpyrazole (580 mg, 2.74 mmol) in THF (13 mL) n-butyllithium (2.4 mL, 2.5 M in THF, 6.0 mmol) was added at −78° C. The mixture was stirred 10 min and was then heated to −30° C. and stirred for 1 h. Acetic acid (200 μL, 3.49 mmol) was added and the mixture was heated to rt. The mixture was filtered through a celite plug, concentrated, and purified by chromatography (SiO₂, PE/EtOAc) to afford the product (288 mg, 75%). ESI-MS m/z calcd for [C₆H₅ClN₂] [M+H]⁺: 141.0; found: 141.2. ¹H NMR (400 MHz, Chloroform-d) δ 7.48-7.41 (m, 1H), 3.12 (s, 1H), 2.05 (d, J=0.9 Hz, 3H).

### Intermediate 42

### 3,4-Dichloro-1H-pyrazole

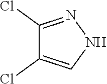

To a solution of 3-chloropyrazole (500 mg, 4.88 mmol) in MeCN (10 mL)N-chlorosuccinimide (665 mg, 4.88 mmol) was added and the mixture was stirred 1 h at 80° C. The mixture was cooled to rt, concentrated, and purified by chromatography (SiO₂, PE/EtOAc) to afford the product (652 mg, 97%). ESI-MS m/z calcd for [C₃H₂Cl₂N₂] [M+H]⁺: 178.0; found: 177.9. H NMR (400 MHz, Chloroform-d) δ 11.08 (s, 1H), 7.61 (s, 1H).

### 3,4-Dichloropyrazole-1-carbaldehyde

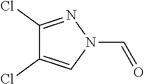

Acetic anhydride (1.47 mL, 15 mmol) was added to formic acid (4.72 mL, 125 mmol) and the mixture was stirred 20 min at rt. The mixture was added to 3,4-dichloro-1H-pyrazole (646 mg, 4.71 mmol) and stirred 1 h at rt. The mixture was concentrated to afford the product (666 mg, 86%). ESI-MS m/z calcd for [C₄H₂Cl₂N₂O] [M+H]⁺: 165.0; found: 164.6. ¹H NMR (400 MHz, Chloroform-d) δ 8.93 (s, 1H), 8.18 (s, 1H).

### 3,4-Dichloro-1-(2,2-dichlorovinyl)pyrazole

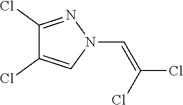 [SMILES: ClC(Cl)=Cn1cc(Cl)c(Cl)n1]

To a solution of 3,4-dichloropyrazole-1-carbaldehyde (666 mg, 4.04 mmol) and triphenylphosphine (3.18 g, 12.1 mmol) in THF (30 mL) carbon tetrachloride (3.9 mL, 40 mmol) was added and the mixture was stirred overnight at 60° C. The mixture was cooled to rt and concentrated. The residue was suspended in diethyl ether/PE, filtered, concentrated, and purified by chromatography (SiO₂, PE/EtOAc) to afford the product (582 mg, 62%). ESI-MS m/z calcd for [C₅H₂Cl₄N₂] [M+H]⁺: 230.9; found: 230.6. ¹H NMR (400 MHz, Chloroform-d) δ 8.13 (s, 1H), 7.32 (s, 1H).

### 3,4-Dichloro-1-ethynylpyrazole

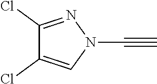

To a solution of 3,4-dichloro-1-(2,2-dichlorovinyl)pyrazole (582 mg, 2.51 mmol) in THF (10 mL) n-butyllithium (2.2 mL, 2.5 M in THF, 5.5 mmol) was added at −78° C. The mixture was stirred 10 min and was then heated to −30° C. and stirred 1 h. Acetic acid (190 μL, 3.2 mmol) was added and the mixture was heated to rt. The mixture was filtered through a celite plug, concentrated, and purified by chromatography (SiO₂, PE/EtOAc) to afford the product (176 mg, 44%). ESI-MS m/z calcd for [C₅H₂Cl₂N₂][M+H]⁺: 161.0; found: 160.8. ¹H NMR (400 MHz, Chloroform-d) δ 7.69 (s, 1H), 3.18 (s, 1H).

### Intermediate 43

### 3-Chloro-4-fluoro-1H-pyrazole

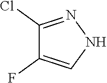

To a solution of 3-chloropyrazole (4.0 g, 39 mmol) in MeCN (80 mL) 1-chloromethyl-4-fluoro-1,4-diazoniabicyclo[2.2.2]octane bis(tetrafluoroborate) (665 mg, 4.88 mmol) was added and the mixture was stirred 24 h at 80° C. The mixture was cooled to rt, filtered over celite and concentrated. The remaining oil was partitioned between water and DCM. The organic phase was dried, concentrated and purified by chromatography (SiO₂, PE/EtOAc). The obtained material was further purified by reversed phase chromatography (Cis, H₂O/MeCN/0.1% TFA) to afford the product (680 mg, 15%). ¹H NMR (400 MHz, Chloroform-d) δ 7.67 (s, 1H).

### 3-Chloro-4-fluoropyrazole-1-carbaldehyde

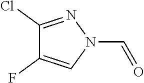

Acetic anhydride (1.25 mL, 13.3 mmol) was added to formic acid (4.2 mL, 110 mmol) and the mixture was stirred 20 min at rt. The mixture was added to 3-chloro-4-fluoro-1H-pyrazole (501 mg, 4.15 mmol) and stirred 1 h at rt. The mixture was concentrated to afford the product (355 mg, 57%). ¹H NMR (400 MHz, Chloroform-d) δ 8.94 (d, J=2.9 Hz, 1H), 8.03 (d, J=5.4 Hz, 1H).

### 3-Chloro-4-fluoro-1-(2,2-dichlorovinyl)pyrazole

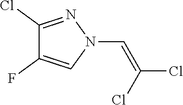 [SMILES: Fc1cn(C=C(Cl)Cl)nc1Cl]

To a solution of 3-chloro-4-fluoropyrazole-1-carbaldehyde (355 mg, 2.39 mmol) and triphenylphosphine (1.88 g, 7.16 mmol) in THF (18 mL) carbon tetrachloride (2.31 mL, 23.7 mmol) was added and the mixture was stirred overnight at 60° C. The mixture was cooled to rt and concentrated. The residue was suspended in diethyl ether/PE, filtered, concentrated, and purified by chromatography (SiO₂, PE/EtOAc) to afford the product (351 mg, 68%). ESI-MS m/z calcd for [C₅H₂Cl₃FN₂] [M+H]⁺: 214.0; found: 215.0. ¹H NMR (400 MHz, Chloroform-d) δ 8.06 (d, J=4.9 Hz, 1H), 7.29 (d, J=1.3 Hz, 1H).

### REFERENCES

Bennett, D.; Bargagli, E.; Bianchi, N.; Landi, C.; Fossi, A.; Fui, A.; Sestini, P.; Refini, R. M.; Rottoli, P. Elevated Level of Galectin-1 in Bronchoalveolar Lavage of Patients with Idiopathic Pulmonary Fibrosis. Respir Physiol Neurobiol 2019, 273, 103323. https://doi.org/10.1016/j.resp.2019.103323.
Blanchard, H.; Yu, X.; Collins, P. M.; Bum-Erdene, K. Galectin-3 Inhibitors: A Patent Review (2008-Present). Expert Opin. Ther. Patents 2014, 24 (10), 1053-1065. https://doi.org/10.1517/13543776.2014.947961.
Blanchard, H.; Bum-Erdene, K.; Bohari, M. H.; Yu, X. Galectin-1 Inhibitors and Their Potential Therapeutic Applications: A Patent Review. Expert Opin. Ther. Patents 2016, 26 (5), 537-554. https://doi.org/10.1517/13543776.2016.1163338.
Daley, D.; Mani, V. R.; Mohan, N.; Akkad, N.; Ochi, A.; Heindel, D. W.; Lee, K. B.; Zambirinis, C. P.; Pandian, G. S. D. B.; Savadkar, S.; Torres-Hernandez, A.; Nayak, S.; Wang, D.; Hundeyin, M.; Diskin, B.; Aykut, B.; Werba, G.; Barilla, R. M.;
Rodriguez, R.; Chang, S.; Gardner, L.; Mahal, L. K.; Ueberheide, B.; Miller, G. Dectin-1 Activation on Macrophages by Galectin-9 Promotes Pancreatic Carcinoma and Peritumoral Immune-Tolerance. Nat Med 2017, 23 (5), 556-567. https://doi.org/10.1038/nm.4314.
Dings, R. P. M., Miller, M. C., Griffin, R. J. & Mayo, K. H. Galectins as Molecular Targets for Therapeutic Intervention. IJMS 19, (2018).
Dubé-Delarosbil, C.; St-Pierre, Y. The Emerging Role of Galectins in High-Fatality Cancers. Cell. Mol. Life Sci. 2017, 75 (7), 1215-1226. https://doi.org/10.1007/s00018-017-2708-5.
Drake, I.; Fryk, E.; Strindberg, L.; Lundqvist, A.; Rosengren, A. H.; Groop, L.;
Ahlqvist, E.; Bordn, J.; Orho-Melander, M.; Jansson, P.-A. The Role of Circulating Galectin-1 in Type 2 Diabetes and Chronic Kidney Disease: Evidence from Cross-Sectional, Longitudinal and Mendelian Randomisation Analyses. Diabetologia 2022, 65 (1), 128-139. https://doi.org/10.1007/s00125-021-05594-1.
Hsu, Y.-A.; Chang, C.-Y.; Lan, J.-L.; Li, J.-P.; Lin, H.-J.; Chen, C.-S.; Wan, L.; Liu, F.-T. Amelioration of Bleomycin-Induced Pulmonary Fibrosis via TGF-β-Induced Smad and Non-Smad Signaling Pathways in Galectin-9-Deficient Mice and Fibroblast Cells. J Biomed Sci 2020, 27 (1), 24. https://doi.org/10.1186/s12929-020-0616-8.
Johannes, L.; Jacob, R.; Leffler, H. Galectins at a Glance. J Cell Sci 2018, 131 (9), jcs208884. https://doi.org/10.1242/jcs.208884.
Kathiriya, J. J.; Nakra, N.; Nixon, J.; Patel, P. S.; Vaghasiya, V.; Alhassani, A.; Tian, Z.; Allen-Gipson, D.; Dav, V. Galectin-1 Inhibition Attenuates Profibrotic Signaling in Hypoxia-Induced Pulmonary Fibrosis. Cell Death Discovery 2017, 3, 17010-17013. https://doi.org/10.1038/cddiscovery.2017.10.
Li, P.; Liu, S.; Lu, M.; Bandyopadhyay, G.; Oh, D.; Imamura, T.; Johnson, A. M. F.;
Sears, D.; Shen, Z.; Cui, B.; Kong, L.; Hou, S.; Liang, X.; Iovino, S.; Watkins, S. M.;
Ying, W.; Osborn, O.; Wollam, J.; Brenner, M.; Olefsky, J. M. Hematopoietic-Derived Galectin-3 Causes Cellular and Systemic Insulin Resistance. Cell 2016, 167 (4), 973-984.e12. https://doi.org/10.1016/j.cell.2016.10.025.
Sethi, A.; Sanam, S.; Alvala, R.; Alvala, M. An Updated Patent Review of Galectin-1 and Galectin-3 Inhibitors and Their Potential Therapeutic Applications (2016-Present). Expert Opin Ther Pat 2021, 31 (8), 1-13. https://doi.org/10.1080/13543776.2021.1903430.
Slack, R. J.; Mills, R.; Mackinnon, A. C. The Therapeutic Potential of Galectin-3 Inhibition in Fibrotic Disease. Int J Biochem Cell Biology 2020, 105881. https://doi.org/10.1016/j.biocel.2020.105881.
Sundblad, V., Morosi, L. G., Geffner, J. R. & Rabinovich, G. A. Galectin-1: A Jack-of-All-Trades in the Resolution of Acute and Chronic Inflammation. J. Immunol. 199, 3721-3730 (2017).
Wolf, Y.; Anderson, A. C.; Kuchroo, V. K. TIM3 Comes of Age as an Inhibitory Receptor. Nat Rev Immunol 2020, 20 (3), 173-185. https://doi.org/10.1038/s41577-019-0224-6.
Wu, D.; Kanda, A.; Liu, Y.; Kase, S.; Noda, K.; Ishida, S. Galectin-1 Promotes Choroidal Neovascularization and Subretinal Fibrosis Mediated via Epithelial-Mesenchymal Transition. FASEB J. 2019, 33 (2), 2498-2513. https://doi.org/10.1096/fj.201801227r.
Yang, R.; Sun, L.; Li, C.-F.; Wang, Y.-H.; Yao, J.; Li, H.; Yan, M.; Chang, W.-C.;
Hsu, J.-M.; Cha, J.-H.; Hsu, J. L.; Chou, C.-W.; Sun, X.; Deng, Y.; Chou, C.-K.; Yu,
D.; Hung, M.-C. Galectin-9 Interacts with PD-1 and TIM-3 to Regulate T Cell Death and Is a Target for Cancer Immunotherapy. Nat Commun 2021, 12 (1), 832. https://doi.org/10.1038/s41467-021-21099-2.

1-15. (canceled)

16. A D-galactopyranose compound of formula (1) 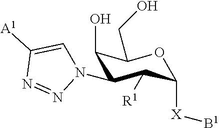
wherein
the pyranose ring is α-D-galactopyranose,
A¹ is 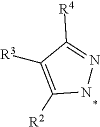
wherein the asterix * indicates the nitrogen atom of the heteroaromatic ring A¹ that is covalently attached to the triazole group of formula (1);
R² is selected from the group consisting of H; halogen; OH; CN; SH; S—C₁₋₆ alkyl; C₁₋₆ alkyl, optionally substituted with a F; cyclopropyl, optionally substituted with a F; O-cyclopropyl optionally substituted with a F; OC₁₋₆ alkyl optionally substituted with a F; NR²⁴R²⁵, wherein R²⁴ is selected from H and C₁₋₆ alkyl, and R²⁵ is selected from H, C₁₋₃ alkyl, and C(═O)R²⁶, wherein R²⁶ is selected from H, and C₁₋₆ alkyl; C(═O)NR^{24a}R^{25a}, wherein R^{24a} is selected from H and C₁₋₆ alkyl, and R^{25a} is selected from H, C₁₋₃ alkyl, and C(═O)R^{26a}, wherein R^{26a} is selected from H, and C₁₋₆ alkyl; C(═O)OR^{24b}R^{25b}, wherein R^{24b} is selected from H and C₁₋₆ alkyl, and R^{25b} is selected from H, C₁₋₃ alkyl, and C(═O)R^{26b}, wherein R^{26b} is selected from H, and C₁₋₆ alkyl;
R³ is selected from the group consisting of H; halogen; OH; CN; SH; S—C₁₋₆ alkyl; C₁₋₆ alkyl, optionally substituted with a F; cyclopropyl, optionally substituted with a F; O-cyclopropyl optionally substituted with a F; OC₁₋₆ alkyl optionally substituted with a F; NR²⁴R²⁵, wherein R²⁴ is selected from H and C₁₋₆ alkyl, and R²⁵ is selected from H, C₁₋₃ alkyl, and C(═O)R²⁶, wherein R²⁶ is selected from H, and C₁₋₆ alkyl; C(═O)NR^{24a}R^{25a}, wherein R^{24a} is selected from H and C₁₋₆ alkyl, and R^{25a} is selected from H, C₁₋₃ alkyl, and C(═O)R^{26a}, wherein R^{26a} is selected from H, and C₁₋₆ alkyl; C(═O)OR^{24b}R^{25b}, wherein R^{24b} is selected from H and C₁₋₆ alkyl, and R^{25b} is selected from H, C₁₋₃ alkyl, and C(═O)R^{26b}, wherein R^{26b} is selected from H, and C₁₋₆ alkyl;
R⁴ is selected from the group consisting of H; halogen; OH; CN; SH; S—C₁₋₆ alkyl; C₁₋₆ alkyl, optionally substituted with a F; cyclopropyl, optionally substituted with a F; O-cyclopropyl optionally substituted with a F; OC₁₋₆ alkyl optionally substituted with a F; NR²⁴R²⁵, wherein R²⁴ is selected from H and C₁₋₆ alkyl, and R²⁵ is selected from H, C₁₋₃ alkyl, and C(═O)R²⁶, wherein R²⁶ is selected from H, and C₁₋₆ alkyl; C(═O)NR^{24a}R^{25a}, wherein R^{24a} is selected from H and C₁₋₆ alkyl, and R^{25a} is selected from H, C₁₋₃ alkyl, and C(═O)R^{26a}, wherein R^{26a} is selected from H, and C₁₋₆ alkyl; C(═O)OR^{24b}R^{25b}, wherein R^{24b} is selected from H and C₁₋₆ alkyl, and R^{25b} is selected from H, C₁₋₃ alkyl, and C(═O)R^{26b} wherein R^{26b} is selected from H, and C₁₋₆ alkyl;
X is selected from S, Se, SO, SO₂, O, C═O, and CR^{2a}R^{3a} wherein R^{2a} and R^{3a} are independently selected from hydrogen, OH, or halogen;
B¹ is selected from a) a C₁₋₆ alkyl or branched C₃₋₆ alkyl substituted with a five or six membered heteroaromatic ring, optionally substituted with a substituent selected from CN, a halogen, methyl optionally substituted with a F, OCH₃ optionally substituted with a F, OCH₂CH₃ optionally substituted with a F, OH, and R^{4a}—CONH— wherein R^{4a} is selected from C₁₋₃ alkyl and cyclopropyl; or a C₁₋₆ alkyl substituted with a phenyl, optionally substituted with a substituent selected from CN, a halogen, methyl optionally substituted with a F, OCH₃ optionally substituted with a F, OCH₂CH₃ optionally substituted with a F, OH, and R^{5a}—CONH— wherein R^{5a} is selected from C₁₋₃ alkyl and cyclopropyl; b) an aryl optionally substituted with a group selected from a halogen; a spiro heterocycle; C₂-alkynyl; CN; —COOH; COOC₁₋₄ alkyl; —CONR⁶R⁷, wherein R⁶ and R⁷ are independently selected from H, C₁₋₃ alkyl, cyclopropyl, and iso-propyl, or R⁶ and R⁷ together with the nitrogen form a heterocycloalkyl; C₁₋₃ alkyl, optionally substituted with a F; cyclopropyl, optionally substituted with a F; isopropyl, optionally substituted with a F; SC₁₋₃ alkyl, optionally substituted with a F; OC₁₋₃ alkyl, optionally substituted with a F; O-cyclopropyl, optionally substituted with a F; O-isopropyl, optionally substituted with a F; NR⁸R⁹, wherein R⁸ and R⁹ are independently selected from H, C₁₋₃ alkyl and isopropyl; OH; and R¹⁰—CONH— wherein R¹⁰ is selected from C₁₋₃ alkyl and cyclopropyl; an aryl; and a heterocycle c) a C₅₋₇ cycloalkyl, optionally substituted with a substituent selected from a halogen, C₂-alkynyl, CN, methyl optionally substituted with a F, OCH₃ optionally substituted with a F, OCH₂CH₃ optionally substituted with a F, OH, and R¹¹—CONH— wherein R¹¹ is selected from C₁₋₃ alkyl and cyclopropyl; and d) a heterocycle optionally substituted with a group selected from a halogen; a spiro heterocycle; C₂-alkynyl; CN; —COOH; COOC₁₋₄ alkyl; —CONR¹²R¹³, wherein R¹² and R¹³ are independently selected from H, C₁₋₃ alkoxy, branched C₃₋₆ alkyl, C₁₋₆ alkyl optionally substituted with a F, bicyclopentanyl, CH₂-cyclopropyl, and CH₂-cyclobutyl, or R¹² and R¹³ together with the nitrogen form a heterocycloalkyl; C₁₋₃ alkyl, optionally substituted with a F; cyclopropyl, optionally substituted with a F; isopropyl, optionally substituted with a F; SC₁₋₃ alkyl, optionally substituted with a F; OC₁₋₃ alkyl, optionally substituted with a F; O-cyclopropyl, optionally substituted with a F; O-isopropyl, optionally substituted with a F; SC₁₋₃ alkyl, optionally substituted with a F; NR¹⁴R¹⁵, wherein R¹⁴ and R are independently selected from H, C₁₋₃ alkyl and isopropyl; OH; an aryl; a heterocycle; and R¹⁶—CONH— wherein R¹⁶ is selected from C₁₋₃ alkyl and cyclopropyl; e) a C₁₋₆ alkyl or branched C₃₋₆ alkyl; f) C₂_₆ alkynyl
R¹ is selected from the group consisting of a) H, b) OH, c) OC₁₋₆ alkyl optionally substituted with one or more halogen, phenyl, phenyl substituted with one or more groups selected form OH and halogen, CN, OR¹⁷, NR¹⁸R¹⁹, and CONH₂, wherein R¹⁷ is selected from the group consisting of H, CN, a halogen, methyl optionally substituted with a F, OCH₃ optionally substituted with a F, OCH₂CH₃ optionally substituted with a F, OH, and R²⁰—CONH— wherein R²⁰ is selected from C₁₋₃ alkyl and cyclopropyl, R¹⁸ is selected from the group consisting of H, CN, a halogen, methyl optionally substituted with a F, OCH₃ optionally substituted with a F, OCH₂CH₃ optionally substituted with a F, OH, and R²¹—CONH— wherein R²¹ is selected from C₁₋₃ alkyl and cyclopropyl, and R¹⁹ is selected from the group consisting of H, CN, a halogen, methyl optionally substituted with a F, OCH₃ optionally substituted with a F, OCH₂CH₃ optionally substituted with a F, OH, and R²²—CONH— wherein R²² is selected from C₁₋₃ alkyl and cyclopropyl, d) branched OC₃₋₆ alkyl optionally substituted with one or more halogen, CN, OR²³, NR²⁴R²⁵, and CONH₂, wherein R²³ is selected from the group consisting of H, CN, a halogen, methyl optionally substituted with a F, OCH₃ optionally substituted with a F, OCH₂CH₃ optionally substituted with a F, OH, and R²⁶—CONH— wherein R²⁶ is selected from C₁₋₃ alkyl and cyclopropyl, R²⁴ is selected from the group consisting of H, CN, a halogen, methyl optionally substituted with a F, OCH₃ optionally substituted with a F, OCH₂CH₃ optionally substituted with a F, OH, and R²⁷—CONH— wherein R²⁷ is selected from C₁₋₃ alkyl and cyclopropyl, and R²⁵ is selected from the group consisting of H, CN, a halogen, methyl optionally substituted with a F, OCH₃ optionally substituted with a F, OCH₂CH₃ optionally substituted with a F, OH, and R²⁸—CONH— wherein R²⁸ is selected from C₁₋₃ alkyl and cyclopropyl, and e) cyclic OC₃₋₆ alkyl optionally substituted with one or more halogen, CN, OR²⁹, NR³⁰R³¹, and CONH₂, wherein R²⁹ is selected from the group consisting of H, CN, a halogen, methyl optionally substituted with a F, OCH₃ optionally substituted with a F, OCH₂CH₃ optionally substituted with a F, OH, and R³²—CONH— wherein R³² is selected from C₁₋₃ alkyl and cyclopropyl, R³⁰ is selected from the group consisting of H, CN, a halogen, methyl optionally substituted with a F, OCH₃ optionally substituted with a F, OCH₂CH₃ optionally substituted with a F, OH, and R³³—CONH— wherein R³³ is selected from C₁₋₃ alkyl and cyclopropyl, and R³¹ is selected from the group consisting of H, CN, a halogen, methyl optionally substituted with a F, OCH₃ optionally substituted with a F, OCH₂CH₃ optionally substituted with a F, OH, and R³⁴—CONH— wherein R³⁴ is selected from C₁₋₃ alkyl and cyclopropyl; or
a pharmaceutically acceptable salt or solvate thereof.

17. The compound of claim 16 wherein R² is hydrogen, C₁₋₃ alkyl or halogen.

18. The compound of claim 16 wherein R³ is hydrogen, C₁₋₃ alkyl or halogen.

19. The compound of claim 16 wherein R⁴ is a halogen or C₁₋₃ alkyl optionally substituted with a F.

20. The compound of claim 16 wherein X is selected from S.

21. The compound of claim 16 wherein B1 is selected from a heteroaryl, optionally substituted with a group selected from a halogen; C₂-alkynyl; CN; methyl optionally substituted with a F; a spiro heterocycle; SC₁₋₃ alkyl, optionally substituted with a F; a CONR¹²R¹³, wherein R¹² and R¹³ are independently selected from H, C₁₋₃ alkyl, cyclopropyl, and iso-propyl or R¹² and R¹³ together with the nitrogen form a heterocycloalkyl; and a heterocycle.

22. The compound of claim 16 wherein B1 is selected from a pyridinyl, optionally substituted with a group selected from a Cl; Br; F; ethynyl; N-(2-oxa)-6-azaspiro[3.3]heptanyl; CO-azetidinyl; CONHCH₃; CONHCH₂CH₃; CON(CH(CH₃)₂)(CH₂CH₃); CON(isobutyl)₂; CON(CH₃)(CH₂C(CH₃)₂F); CON(CH₂CH₃)(CH₂C(CH₃)₂F); CON(CH₂CH₃)(CH₂-cyclopropyl); CON(CH₂CH₃)(tert-butyl); CON(CH₂-cyclopropyl)₂; CON(CH₂CH₃)(CH₂-cyclobutyl); CON(CH(CH₃)₂)(CH₂-cyclobutyl); CON(CH₂-cyclobutyl)₂; CON(CH₂CH₃)(CH₂CF₃); CON(CH(CH₃)₂)(CH₂-cyclopropyl); CON(CH(CH₃)₂)(isobutyl); CON(CH₃)₂; CO-pyrrolidinyl; CON(OCH₃)(CH₂-cyclopropyl); CONHCH₂CH₂CH₂CH₃; CONH(isobutyl); CONH(CH₂CH₂F); CONH(bicyclopentanyl); CONH(cyclopropyl); CONH(cyclobutyl); CN; methyl; SCH₃; SCF₃; CF₃; imidazole; pyridin; pyrimidin; oxazol; and thiazol.

23. The compound of claim 16 wherein B1 is selected from a phenyl, optionally substituted with a group selected from a halogen; CN; —CONR⁶R⁷, wherein R⁶ and R⁷ are independently selected from H, C₁₋₃ alkyl, cyclopropyl, and iso-propyl; and C₁₋₃ alkyl, optionally substituted with a F.

24. The compound of claim 16 wherein B1 is selected from a phenyl, substituted with two groups selected from a Cl; F; Br; CN; CONHCH₃; and C₁₋₃ alkyl, optionally substituted with a F.

25. The compound of claim 16 wherein R¹ is selected from H, OH, OC₁₋₄ alkyl, OC₁₋₄ alkyl substituted with at least one from the group consisting of phenyl and phenyl substituted with one or more groups selected form OH and halogen.

26. The compound of claim 16 wherein R¹ is selected from OH and OCH₃.

27. The compound of claim 16 selected from the group consisting of:
5-Bromo-2-(N-methyl-carbonyl)phenyl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-cyanophenyl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-cyanopyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Chloropyridin-3-yl 3-deoxy-3-[4-(3-fluoro-1H-1,2-pyrazol-1-yl)-1H-1,2,3-triazol-1-yl]-1-thio-α-D-galactopyranoside,
5-Chloropyridin-3-yl 3-[4-(3-chloro-1H-1,2-pyrazol-1-yl)-1H-1,2,3-triazol-1-yl]-3-deoxy-1-thio-α-D-galactopyranoside,
5-Chloro-2-(trifluoromethyl)pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-(trifluoromethyl)pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
3-Chloro-2-(trifluoromethyl)pyridin-5-yl 3-[4-(3-chloro-1H-1,2-pyrazol-1-yl)-1H-1,2,3-triazol-1-yl]-3-deoxy-2-O-methyl-1-thio-α-D-galactopyranoside,
3-Bromo-2-(trifluoromethyl)pyridin-5-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-(N,N-dimethylcarbamoyl)pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-cyanopyridin-3-yl 3-[4-(4-chloro-1H-1,2-pyrazol-1-yl)-1H-1,2,3-triazol-1-yl]-3-deoxy-2-O-methyl-1-thio-α-D-galactopyranoside,
5-Bromo-2-cyanopyridin-3-yl 3-deoxy-3-[4-(3-fluoro-1H-1,2-pyrazol-1-yl)-1H-1,2,3-triazol-1-yl]-2-O-methyl-1-thio-α-D-galactopyranoside,
5-Bromo-2-cyanopyridin-3-yl 3-deoxy-3-[4-(4-fluoro-1H-1,2-pyrazol-1-yl)-1H-1,2,3-triazol-1-yl]-2-O-methyl-1-thio-α-D-galactopyranoside,
5-Bromo-2-cyanopyridin-3-yl 3-deoxy-3-[4-(3-methyl-1H-1,2-pyrazol-1-yl)-1H-1,2,3-triazol-1-yl]-2-O-methyl-1-thio-α-D-galactopyranoside,
5-Bromo-2-cyanopyridin-3-yl 3-deoxy-3-[4-(5-methyl-1H-1,2-pyrazol-1-yl)-1H-1,2,3-triazol-1-yl]-2-O-methyl-1-thio-α-D-galactopyranoside,
5-Bromo-2-cyanopyridin-3-yl 3-[4-(3-chloro-5-methyl-1H-1,2-pyrazol-1-yl)-1H-1,2,3-triazol-1-yl]-3-deoxy-2-O-methyl-1-thio-α-D-galactopyranoside,
5-Bromo-2-cyanopyridin-3-yl 3-{4-[5-chloro-3-(trifluoromethyl)-1H-1,2-pyrazol-1-yl]-1H-1,2,3-triazol-1-yl}-3-deoxy-2-O-methyl-1-thio-α-D-galactopyranoside,
5-Bromo-2-cyanopyridin-3-yl 3-[4-(3-chloro-4-methyl-1H-1,2-pyrazol-1-yl)-1H-1,2,3-triazol-1-yl]-3-deoxy-2-O-methyl-1-thio-α-D-galactopyranoside,
5-Bromo-2-(N,N-ethylisopropylcarbamoyl)pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-(N,N-diisobutylcarbamoyl)pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-I[N,N-(cyclopropylmethyl)ethylcarbamoyl]pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-[N,N-(2-fluoro-2-methylpropyl)methylcarbamoyl]pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-[N,N-(tert-butyl)ethylcarbamoyl]pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-[N,N-bis(cyclopropylmethyl)carbamoyl]pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-[N,N-(cyclobutylmethyl)ethylcarbamoyl}pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-[N,N-(cyclobutylmethyl)isopropylcarbamoyl]pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-[N,N-bis(cyclobutylmethyl)carbamoyl]pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-(pyrrolidine-1-carbonyl)pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-[N,N-ethyl(2,2,2-trifluoroethyl)carbamoyl]pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-[N,N-ethyl(2-fluoro-2-methylpropyl)carbamoyl]pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-[N,N-(cyclopropylmethyl)isopropylcarbamoyl]pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-(N,N-isobutylisopropylcarbamoyl)pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-[N,N-(cyclopropylmethyl)methoxycarbamoyl]pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-(N-methylcarbamoyl)pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-(N-ethylcarbamoyl)pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-(N-butylcarbamoyl)pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-(N-isobutylcarbamoyl)pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-[N-(2-fluoroethyl)carbamoyl]pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-(N-bicyclo[1.1.1]pentan-1-ylcarbamoyl)pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-(N-cyclobutylcarbamoyl)pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-(N-cyclopropylcarbamoyl)pyridin-3-yl 3-[4-(3-chloro-1H-1,2-pyrazol-1-yl)-1H-1,2,3-triazol-1-yl]-3-deoxy-2-O-methyl-1-thio-α-D-galactopyranoside,
5-Bromo-2-cyanopyridin-3-yl 3-[4-(3,4-dichloro-1H-1,2-pyrazol-1-yl)-1H-1,2,3-triazol-1-yl]-3-deoxy-2-O-methyl-1-thio-α-D-galactopyranoside,
5-Bromo-2-cyanopyridin-3-yl 3-[4-(3-chloro-4-fluoro-1H-1,2-pyrazol-1-yl)-1H-1,2,3-triazol-1-yl]-3-deoxy-2-O-methyl-1-thio-α-D-galactopyranoside; or
a pharmaceutically acceptable salt or solvate thereof.

28. The compound of claim 16 for use as a medicine.

29. A pharmaceutical composition comprising the compound of claim 16 and optionally a pharmaceutically acceptable additive.

30. A method for treating a disorder relating to the binding of a galectin-1 and/or a galectin 3 to a ligand in a mammal, wherein said disorder is selected from the group consisting of inflammation, acute post myocardial infarctions (MI), acute coronary syndrome, acute stent occlusion, acute myocardial reperfusion injury, acute pneumonitidies, acute lung injury (ALI), acute kidney injury (AKI), acute hepatitis, acute on chronic liver failure, acute alcohol hepatitis, acute pancreatitis, acute uveitis, acute pancreatitis related liponecrosis, acute retinitis, acute nephritis, acute myocarditis, chronic autoimmune diseases in all organs, chronic autoimmune diseases in lung, liver, kidney, heart, skin, muscle, and/or gut, chronic bacterial infections, chronic viral related inflammation; fibrosis, pulmonary fibrosis, liver fibrosis, kidney fibrosis, ophthalmological fibrosis and fibrosis of the skin and heart, acute post-surgical ocular fibrosis, acute transplantation rejection of the kidney, heart, lung, liver, and pancreas, acute post explosion/improvised explosive devices, acute post toxic dust, acute chemical exposure, chronic lung fibrosis, interstitial lung fibrosis (IPF), Interstitial Lung Disease (ILD), Childhood ILD (ChILD); chronic liver fibrosis, chronic alcohol fibrosis, chronic viral fibrosis, chronic diabetic fibrosis, diabetic nephropathy, chronic glomerulonephritis, renal artery stenosis, endometriosis; scarring; keloid formation; aberrant scar formation; surgical adhesions; scleroderma; systemic sclerosis; septic shock; cancers, carcinomas, sarcomas, leukemias and lymphomas, T-cell lymphomas; metastasising cancers; autoimmune diseases, psoriasis, rheumatoid arthritis, Crohn's disease, ulcerative colitis, intestinal fibrosis, ankylosing spondylitis, systemic lupus erythematosus; metabolic disorders; coagulopathies, thrombosis proneness idiopathic (thrombophilia), autoimmune based thrombophilia, microthrombosis at multiorgan failure, COVID-19 related coagulopathy, thrombophilia in cancer disease; cardiovascular disorders, cardiac fibrosis, cardiac failure, left and right atrial fibrillation, atheromatosis, arterial inflammation, arterial calcification, aortic stenosis; heart disease; heart failure; aortic stenosis, atherosclerosis, pathological angiogenesis, ocular angiogenesis or a disease or condition associated with ocular angiogenesis, e.g. neovascularization related to cancer; and eye diseases, age-related macular degeneration and corneal neovascularization; atherosclerosis; endocrine disorders, Addison, autoimmune hypophysitis; metabolic diseases, diabetes; type 2 diabetes; insulin resistance; obesity; Diastolic HF; atrophic diseases in the brain, Alzheimer's and Parkinson's, atrophic diseases in the cerebellum, cerebellar atrophy, atrophic spinal diseases, ALS; disorders related to transplantation in organs, anti-rejection prophylaxis, anti-acute rejection, anti-chronic rejection; acute burn; acute inflammatory reaction; chronic acute skin graft rejection; chronic scarring; asthma and other interstitial lung diseases, including Hermansky-Pudlak syndrome, pulmonary arterial hypertension, Rheumatoid disease associated interstitial lung disease RA-ILD, Systemic Sclerosis SSc-ILD, lung disease with fibrosis, COPD (Chronic Obstructive Pulmonary Disease) and asthma; Otosclerosis, mesothelioma; post-surgery disorders, anti-keloid, anti-stricture, anti-adhesion, anti-thrombosis, fibrosis/scar reduction following cosmetic procedures; toxin exposure disorders, toxic hepatitis, cholera toxin related, mushroom toxin based acute renal failure, pertussis toxin, Aeromonas hydrophila enterotoxin, cadmium induced cardiac toxicity, helicobacter O-antigen related toxicity, LPS based toxicity, Streptozotocin toxicity, asbestos exposure, Nephrogenic Systemic Fibrosis (Post Contrast Agents); Tissue injury, Spinal cord injury, Peripheral nerve repair; congenital hepatic fibrosis; hereditary fibrosing poikiloderma with tendon contractures, myopathy, and pulmonary fibrosis; liver disorders, non-alcoholic steatohepatitis (NASH) or non-alcoholic fatty liver disease, liver cirrhosis of various origins, alcoholic and non-alcoholic, autoimmune cirrhosis primary biliary cirrhosis and sclerosing cholangitis, virally induced cirrhosis, cirrhosis induced by genetic disease; Liver cancer, cholangiocarcinoma, biliary tract cancer; neurodegenerative disorders Parkinsons disease, Alzheimers disease, cognitive impairment, cerebrovascular diseases, stroke, traumatic brain injury, Huntington's disease, amyotrophic lateral sclerosis (ALS), multiple sclerosis (MS), peripheral nephropathy, comprising administering a therapeutically effective amount of the compound of claim 16.
